(19) United States
(12) Patent Application Publication
(10) Pub. No.: US 2017/0267768 A1
(43) Pub. Date: Sep. 21, 2017

(54) Protein A Binding Polypeptides, Anti-EphA2 Antibodies and Methods of Use Thereof

(71) Applicants: The Regents of the University of California, Oakland, CA (US); Merrimack Pharmaceuticals, Inc., Cambridge, MA (US)

(72) Inventors: James D. Marks, Kensington, CA (US); Daryl Drummond, Lincoln, MA (US); Melissa Geddie, Arlington, MA (US); Dmitri Kirpotin, Revere, MA (US); Neeraj Kohli, Arlington, MA (US); Alexey Lugovskoy, Belmont, MA (US)

(21) Appl. No.: 15/457,857

(22) Filed: Mar. 13, 2017

Related U.S. Application Data
(60) Provisional application No. 62/309,365, filed on Mar. 16, 2016.
(60) Provisional application No. 62/309,383, filed on Mar. 16, 2016.
(60) Provisional application No. 62/309,374, filed on Mar. 16, 2016.

(51) Int. Cl. C07K 16/28; C07K 16/12

(52) U.S. Cl. CPC C07K 16/2866; C07K 16/1271; C07K 2317/565; C07K 2317/21; C07K 2317/24; C07K 2317/567; C07K 2317/622; C07K 2317/94; C07K 2317/76

(57) ABSTRACT

Provided by the present disclosure are antibodies (e.g., scFvs) that include CDRs and human framework regions that confer useful properties upon the antibodies. In certain embodiments, such properties include thermostability (e.g., increased melting temperature), efficient binding to Staphylococcus aureus Protein A, or both. In certain aspects, the antibodies are internalizing antibodies that specifically bind to the tumor associated antigen EphA2.

### CROSS REFERENCE TO RELATED APPLICATIONS

This application claims priority to U.S. Provisional Application No. 62/309,365, filed Mar. 16, 2016, and to U.S. Provisional Application No. 62/309,383, filed Mar. 16, 2016, and to U.S. Provisional Application No. 62/309,374, filed Mar. 16, 2016, each of which is incorporated herein by reference in its entirety.

### INTRODUCTION

Cancer cells often aberrantly express a variety of proteins, including expression or overexpression of certain cell surface antigens, e.g., cell surface receptors. Such aberrantly expressed proteins can be exploited as targets for therapeutic antibodies, and antibodies specific to cell surface receptors expressed on a number of cancers have been utilized for development of targeted immunotherapeutics. Many such therapeutic agents, including antibody-drug conjugates, (including immunoliposomes and immunotoxins), and antibody-targeted nucleic acid delivery vehicles, require antibodies that not only bind the cell surface antigen, but undergo internalization into the cell upon binding. For example, antibodies recognizing cell surface receptors expressed by cancer cells that have been conjugated with chemotherapeutic agents are currently used to treat Hodgkin lymphoma (brentuximab vedotin) and metastatic breast cancer (ado-trastuzumab emtansine).

One cell surface receptor being considered for therapeutic targeting, as it is known to be aberrantly expressed in several cancers, is EphA2. As described in U.S. Pat. No. 9,220,772, anti-EphA2 scFv antibodies were isolated using a multistep screening process that involved first a general phage selection for internalizing antibodies using human breast cancer cells, followed by selections for binding to yeast expressing the extracellular domain of EphA2. Antibody D2-1A7 was identified as a ligand blocking scFv that is strongly internalized by cells expressing EphA2.

Antibody stability is a useful property, allowing for a robust and scalable antibody manufacturing process. Additionally, to facilitate the manufacturing of an antibody for therapeutic use, it is desirable that the antibody be capable of purification with an affinity resin. For most therapeutic antibodies, Staphylococcus aureus Protein A affinity chromatography is an early and important step because it can selectively and efficiently bind antibodies from complex solutions such as harvested cell culture fluids removing >99.5% of product impurities in a single step and providing significant viral clearance.

There is a continuing need for new antibodies that bind to cell surface receptors and exhibit stability and other manufacturability properties suitable for industrial production and medical use. The novel engineered antibodies disclosed herein address this need and provide additional advantages.

### SUMMARY

The present disclosure provides antibodies (e.g., scFvs) that include CDRs and human framework regions that confer useful properties upon the antibodies. In certain embodiments, such properties include thermostability (e.g., increased melting temperature), efficient binding to Staphylococcus aureus Protein A, or both. In certain aspects, the antibodies are internalizing antibodies that specifically bind to the tumor associated antigen EphA2.

The present disclosure provides internalizing anti-EphA2 antibodies (Abs) (e.g., scFvs) that are thermo-stable (exhibiting a melting temperature of 267° C. or 270° C.). Also provided are Abs (e.g, scFvs) that exhibit substantial binding to Staphylococcus aureus Protein A (e.g., to an extent such that at least 50%, 60%, or 70% of Ig loaded onto S. aureus Protein A (hereinafter “Protein A”) resin binds to and is retained on the resin, e.g., using the assay of Example 1). Polypeptides conferring the desired Protein A binding properties, as well as antibodies (e.g., scFv) containing such Protein A-binding polypeptides; VH CDR2 polypeptides conferring desired Protein A binding properties, well as antibodies (e.g., scFv) containing such; VH and VL framework polypeptides (including consensus framework sequences) providing desired thermostability properties, as well as antibodies (e.g., scFv) having such framework sequence; and Abs (e.g., scFvs) having combinations of such Protein-A binding polypeptides, VH CDR2 polypeptides and thermostability enhancing frameworks are provided. In addition, the disclosed Abs exhibit other properties desirable for commercial scale liposome conjugation and other manufacturing processes, as well as for therapeutic uses.

Thus, anti-EphA2 antibodies designed and adapted for commercial manufacture and for medical uses are disclosed, as well as related conjugates, compositions and methods of use. Methods of use encompass use as targeting components for liposomal therapeutics, as well as use as diagnostics, and in screening methods.

Accordingly, the present disclosure provides an anti-Ephrin type-A receptor 2 (EphA2) antibody comprising:
a VH comprising:
   a VH CDR1 comprising the amino acid sequence SYAMH (SEQ ID NO: 17);
   a VH CDR2 comprising the following sequence of 17 amino acids:
      X¹⁷ISPX¹⁸GX¹⁹NX²⁰YYADSVKG (SEQ ID NO: 18), wherein:
         X¹⁷ is A, or V;
         X¹⁸ is A, D, H, N, P, S or T;
         X¹⁹ is A, R, H, N or P; and
         X²⁰ is K or T; and
   a VH CDR3 comprising the amino acid sequence ASVGATGPFDI (SEQ ID NO: 19); and
a VL comprising:
   a VL CDR1 comprising the amino acid sequence QGDSLRSYYAS (SEQ ID NO: 20);
   a VL CDR2 comprising the amino acid sequence GENNRPS (SEQ ID NO: 21); and
   a VL CDR3 comprising the amino acid sequence NSRDSSGTHLTV (SEQ ID NO: 22);
      wherein the antibody immunospecifically binds to the extracellular domain of EphA2.

The present disclosure also provides polypeptides that bind Protein A, wherein the polypeptide comprises an amino acid sequence of the formula:

a) X^{a}X¹⁷ISX^{b}X¹⁸GX¹⁹NX²⁰ (SEQ ID NO: 198), wherein
X^{a} is S, T or A;
X¹⁷ is A, or V;
X^{b} is P, R, N or H,
X¹⁸ and X¹⁹ are any amino acid, and in some embodiments X¹⁸ is A, D, H, N, P, S or T; and/or wherein X¹⁹ is A, R, H, N or P; and
X²⁰ is K or T;

b) X^{a}VISX^{b}X¹⁸GX¹⁹NX²⁰ (SEQ ID NO: 199), wherein
X^{a} is S or T,
X^{b} is P, R, or N,
X¹⁸ is any amino acid, and in some embodiment is A, D, H, N, P, S or T;
X¹⁹ is H, N, R, T, A, D, P, or S; and
X²⁰ is K or T;

c) X^{a}VISX^{b}X¹⁸GX¹⁹NX²⁰ (SEQ ID NO: 200), wherein
X^{a} is S or T,
X^{b} is P, R, or N,
X¹⁸ is any amino acid, and in some embodiment is A, D, H, N, P, S or T;
X¹⁹ is H, N, R, or T; and
X²⁰ is T or K;
or

d) X^{a}X¹⁷ISX^{b}X¹⁸GX¹⁹NX²⁰ (SEQ ID NO: 201), wherein
X^{a} is S or T,
X¹⁷ is V;
X^{b} is P,
X¹⁸ is any amino acid, and in some embodiment is A, D, H, N, P, S or T;
X¹⁹ is H, N, R, or T, and in some embodiments is A, R, H, N or P; and
X²⁰ is T or K.

In related embodiments, the Protein A binding polypeptide comprises the amino acid sequence of one of SEQ ID NOS: 177-197.

In some embodiments, the Protein A binding polypeptides comprise:

a VH CDR1 comprising an amino acid sequence of SYAMH (SEQ ID NO:17);

a Protein A binding sequence comprising the amino acid sequence of one of SEQ ID NOs: 177 to 201; and

a VH CDR3 comprising an amino acid sequence of ASVGATGPFDI (SEQ ID NO: 19).

The present disclosure also provides antibodies comprising a VH and a VL, wherein the VH comprises a Protein A binding polypeptide as described hereinabove.

In certain embodiments, the antibody comprises at least one framework region of:
(a) a human VH framework region 1 (VH FR1) comprising the amino acid sequence QVQLVX¹SGGGLVQPGGSLRLSCAASGFTFS (SEQ ID NO: 1), wherein X¹ is an amino acid other than E, and/or
(b) a human VH framework region 2 (VH FR2) comprising the amino acid sequence WVRQAPGKGLEWVX² (SEQ ID NO: 2), wherein X² is an amino acid other than S; or, where the antibody comprises a Protein A binding polypeptide comprising an amino acid sequence of one of SEQ ID NOs: 177-201, a human VH framework region 2 (VH FR2) comprising the amino acid sequence WVRQAPGKGLEWV (SEQ ID NO: 202), and/or
(c) a human VL framework region 1 (VL FR1) comprising the amino acid sequence X³SX⁴LTQPPSVSX⁵APGQX⁶VTIX⁷C (SEQ ID NO: 3), wherein: X³ is an amino acid other than Q, X⁴ is an amino acid other than V, X⁵ is an amino acid other than G, X⁶ is an amino acid other than R, and X⁷ is an amino acid other than S, and/or
(d) a human VL framework region 1 (VL FR1) comprising the amino acid sequence X⁸SVLTQPPSVSGAPGQRVTIX⁹C (SEQ ID NO: 4), wherein: X⁸ is an amino acid other than Q, or X⁹ is an amino acid other than S, and/or
(e) a human VL framework region 2 (VL FR2) comprising the amino acid sequence WYQQX¹⁰PGTAPKLLIY (SEQ ID NO: 5), wherein X¹⁰ is an amino acid other than L, and/or
(f) a human VL framework region 3 (VL FR3) comprising the amino acid sequence GVPDRFSGX¹¹X¹²SGTSASLX¹³ITGX¹⁴QAEDEADYYC (SEQ ID NO: 6), wherein: X¹¹ is an amino acid other than F, X¹² is an amino acid other than K, X¹³ is an amino acid other than A, or X¹⁴ is an amino acid other than L, and/or
(g) a human VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, or X¹⁶ is an amino acid other than K.

In certain embodiments, the antibody comprises a VH FR1 comprising the amino acid sequence QVQLVQSGGGLVQPGGSLRLSCAASGFTFS (SEQ ID NO: 8). In certain embodiments, the antibody comprises a VH FR2 comprising the amino acid sequence WVRQAPGKGLEWVA (SEQ ID NO: 9) or WVRQAPGKGLEWVT (SEQ ID NO: 10). In certain embodiments, the antibody comprises a VL FR1 comprising the amino acid sequence X³SX⁴LTQPPSVSX⁵APGQX⁶VTIX⁷C (SEQ ID NO: 3), and wherein X³ is an amino acid other than Q, X⁴ is an amino acid other than V, X⁵ is an amino acid other than G, X⁶ is an amino acid other than R, and X⁷ is an amino acid other than S. In certain embodiments, the antibody comprises a VL FR1 comprising the amino acid sequence X³SX⁴LTQPPSVSX⁵APGQX⁶VTIX⁷C (SEQ ID NO: 3), and wherein X³ is S, X⁴ is E, X⁵ is V, X⁶ is T, and X⁷ is T. In certain embodiments, the antibody comprises a VL FR1 comprising the amino acid sequence X⁸SVLTQPPSVSGAPGQRVTIX⁹C (SEQ ID NO: 4), and wherein X⁸ is an amino acid other than Q, and X⁹ is an amino acid other than S. In certain embodiments, the antibody comprises a VL FR1 comprising the amino acid sequence X⁸SVLTQPPSVSGAPGQRVTIX⁹C (SEQ ID NO: 4), and wherein X⁸ is S or X⁹ is T. In certain embodiments, the antibody comprises a VL FR1 comprising the amino acid sequence X⁸SVLTQPPSVSGAPGQRVTIX⁹C (SEQ ID NO: 4), and wherein X⁸ is S and X⁹ is T. In certain embodiments, the antibody comprises a VL FR2 comprising the amino acid sequence WYQQX¹⁰PGTAPKLLIY (SEQ ID NO: 5), and wherein X¹⁰ is an amino acid other than L. In certain embodiments, the antibody comprises a VL FR2 comprising the amino acid sequence WYQQX¹⁰PGTAPKLLIY (SEQ ID NO: 5), and wherein X¹⁰ is K. In certain embodiments, the antibody comprises a VL FR3 comprising the amino acid sequence GVPDRFSGX¹¹X¹²SGTSASLX¹³ITGX¹⁴QAEDEADYYC (SEQ ID NO: 6), and wherein X¹¹ is an amino acid other than F, X¹² is an amino acid other than K, X¹³ is an amino acid other than A, and X¹⁴ is an amino acid other than L. In certain embodiments, the antibody comprises a VL FR3 comprising the amino acid sequence GVPDRFSGX¹¹X¹²SGTSASLX¹³ITGX¹⁴QAEDEADYYC, (SEQ ID NO: 6) and wherein X¹¹ is S, X¹² is S, X¹³ is T, or X¹⁴ is A. In certain embodiments, the antibody comprises a VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, and X¹⁶ is an amino acid other than K. In certain embodiments, the antibody comprises a VL FR3 comprising the amino acid sequence GVPDRFSGX¹⁵X¹⁶SGTSASLAITGLQAEDEADYYC (SEQ ID NO: 7), wherein: X¹⁵ is S or X¹⁶ is G. In certain embodiments, the antibody comprises a VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is S and X¹⁶ is G.

In certain embodiments, the antibody comprises:

a VH framework region 1 (VH FR1) comprising the amino acid sequence QVQLVX¹SGGGLVQPGGSLRLSCAASGFTFS, (SEQ ID NO: 1) wherein X¹ is an amino acid other than E;

a VH framework region 2 (VH FR2) comprising the amino acid sequence WVRQAPGKGLEWVX² (SEQ ID NO: 2), wherein X² is an amino acid other than S;

a VL framework region 1 (VL FR1) comprising the amino acid sequence X³SX⁴LTQPPSVSX⁵APGQX⁶VTIX⁷C (SEQ ID NO: 3), wherein X³ is an amino acid other than Q, X⁴ is an amino acid other than V, X⁵ is an amino acid other than G, X⁶ is an amino acid other than R, and X⁷ is an amino acid other than S;
a VL framework region 2 (VL FR2) comprising the amino acid sequence WYQQX¹⁰PGTAPKLLIY (SEQ ID NO: 5), wherein X¹⁰ is an amino acid other than L; and

a VL framework region 3 (VL FR3) comprising the amino acid sequence GVPDRFSGX¹¹X¹²SGTSASLX¹³ITGX¹⁴QAEDEADYYC (SEQ ID NO: 6), wherein X¹¹ is an amino acid other than F, X¹² is an amino acid other than K, X¹³ is an amino acid other than A, and X¹⁴ is an amino acid other than L.

In certain embodiments, the antibody comprises:

a VH framework region 1 (VH FR1) comprising the amino acid sequence QVQLVX¹SGGGLVQPGGSLRLSCAASGFTFS (SEQ ID NO: 1), wherein X¹ is an amino acid other than E;

a VH framework region 2 (VH FR2) comprising the amino acid sequence WVRQAPGKGLEWVX² (SEQ ID NO: 2), wherein X² is an amino acid other than S; a VL framework region 1 (VL FR1) comprising the amino acid sequence X⁸SVLTQPPSVSGAPGQRVTIX⁹C (SEQ ID NO: 4), wherein: X⁸ is an amino acid other than Q, or X⁹ is an amino acid other than S; and

a VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, and X¹⁶ is an amino acid other than K.

In certain embodiments, the antibody has a melting temperature (Tₘ) of 67° C. or higher as measured using differential scanning fluorimetry.

In certain embodiments, the antibody is a single chain Fv (scFv), IgG, Fab, (Fab)₂, or (scFv′)₂. In certain embodiments, the antibody is an scFv. In certain embodiments, the antibody comprises human antibody sequences.

In certain embodiments, the antibody is an scFv comprising a VH, a VL, and frameworks, wherein:

the VH comprises:

a VH CDR1 having amino acid sequence SYAMH (SEQ ID NO: 17);

a VH CDR2 having one of the following 18 amino acid sequences:

| (SEQ ID NO: 23) | |
|---|---|
| (I) | VISPAGNNTYYADSVK, |
| (SEQ ID NO: 24) | |
| (II) | VISPAGRNKYYADSVK, |
| (SEQ ID NO: 25) | |
| (III) | VISPDGHNTYYADSVK, |
| (SEQ ID NO: 26) | |
| (IV) | VISPHGRNKYYADSVK, |
| (SEQ ID NO: 27) | |
| (V) | VISRRGDNKYYADSVK, |
| (SEQ ID NO: 28) | |
| (VI) | VISNNGHNKYYADSVK, |
| (SEQ ID NO: 29) | |
| (VII) | VISPAGPNTYYADSVK, |
| (SEQ ID NO: 30) | |
| (VIII) | VISPSGHNTYYADSVK, |
| (SEQ ID NO: 31) | |
| (IX) | VISPNGHNTYYADSVK, |
| (SEQ ID NO: 32) | |
| (X) | AISPPGHNTYYADSVK, |
| (SEQ ID NO: 33) | |
| (XI) | VISPTGANTYYADSVK, |
| (SEQ ID NO: 34) | |
| (XII) | VISPHGSNKYYADSVK, |
| (SEQ ID NO: 35) | |
| (XIII) | VISNNGHNTYYADSVK, |
| (SEQ ID NO: 36) | |
| (XIV) | VISPAGTNTYYADSVK, |
| (SEQ ID NO: 37) | |
| (XV) | VISPPGHNTYYADSVK, |
| (SEQ ID NO: 38) | |
| (XVI) | VISHDGTNTYYADSVK, |
| (SEQ ID NO: 39) | |
| (xvii) | VISRHGNNKYYADSVK, |
| or | |
| (SEQ ID NO: 40) | |
| (xviii) | VISYDGSNKYYADSVK; |

and

a VH CDR3 having amino acid sequence ASVGATGPFDI (SEQ ID NO: 19); wherein the VH CDRs are present in the following amino to carboxyl terminal order: VH CDR1, VH CDR2, VH CDR3,

and

the VL comprises:

a VL CDR1 having amino acid sequence QGDSLRSYYAS (SEQ ID NO: 20),

a VL CDR2 having amino acid sequence GENNRPS (SEQ ID NO: 21), and

a VL CDR3 having amino acid sequence NSRDSSGTHLTV (SEQ ID NO: 22);

and wherein

the VL CDRs are present in the following amino to carboxyl terminal order: VL CDR1, VL CDR2, and VL CDR3;

the scFv frameworks comprise:

| (SEQ ID NO: 8) | |
|---|---|
| VH FR1 | QVQLVQSGGGLVQPGGSLRLSCAASGFTFS |
| (SEQ ID NO: 9) | |
| VH FR2 | WVRQAPGKGLEWVA |
| (SEQ ID NO: 11) | |
| VH FR3 | RFTISRDNSKNTLYLQMNSLRAEDTAVYYCAR |
| (SEQ ID NO: 12) | |
| VH FR4 | WGQGTLVTVSS |
| (SEQ ID NO: 13) | |
| VL FR1 | SSELTQPPSVSVAPGQTVTITC |
| (SEQ ID NO: 14) | |
| VL FR2 | WYQQKPGTAPKLLIY |
| (SEQ ID NO: 15) | |
| VL FR3 | GVPDRFSGSSSGTSASLTITGAQAEDEADYYC |
| and | |
| (SEQ ID NO: 16) | |
| VL FR4 | FGGGTKLTVLG |

and wherein the antibody immunospecifically binds to the extracellular domain of ephrin type-A receptor 2 (EphA2).

In certain embodiments, the of the scFv VH is amino terminal to the VL.

In certain embodiments, the scFv exhibits a Tm of at least about 67° C. In certain embodiments, the scFv exhibits a Tm of at least about 70° C.

In certain embodiments, the scFv comprises a linker joining the VH and VL, wherein the linker comprises an amino acid sequence selected from:

(SEQ ID NO: 41)
ASTGGGGSGGGGSGGGGSGGGGS,
(SEQ ID NO: 42)
GGGGSGGGGSGGGGSGGGGS,
(SEQ ID NO: 43)
GGGGSGGGGSGGGGS,
(SEQ ID NO: 44)
ASTGGGGAGGGGAGGGGAGGGGA,
(SEQ ID NO: 45)
GGGGAGGGGAGGGGAGGGGA,
(SEQ ID NO: 46)
TPSHNSHQVPSAGGPTANSGTSGS,
and
(SEQ ID NO: 47)
GGSSRSSSSGGGGSGGGG.

In certain embodiments, the scFv linker comprises the amino acid sequence ASTGGGGSGGGGSGGGGSGGGGS (SEQ ID NO: 41).

In certain embodiments, the antibody comprises a VH and a VL, where in the VH comprises an amino acid sequence at least 95% identical the amino acid sequence of a VH and a VL, wherein the VH comprises an amino acid sequence having at least 95% amino acid sequence identity to the amino acid sequence of SEQ ID NO: 133, 135, 137, 139, 141, 143, 145, 147, 149, 151, 153, 155, 157, 159, 161, 163, 165, 167, 169, 171, 173, or 175. In related embodiments, the VL of these antibodies can comprise an amino acid sequence having at least 95% amino acid sequence identity to the amino acid sequence of SEQ ID NO: 134, 136, 138, 140, 142, 144, 146, 148, 150, 152, 154, 156, 158, 160, 162, 164, 166, 168, 170, 172, 174, or 176.

In certain embodiments, the antibody comprises, at the C-terminus, Gly Gly Ser Gly Gly Cys (SEQ ID NO: 54).

In certain embodiments, the present disclosure provides an antibody conjugate comprising an antibody as disclosed herein; and a moiety conjugated to the antibody. In certain embodiments, the moiety is a lipidic nanoparticle. In certain embodiments, the lipidic nanoparticle is a liposome. In certain embodiments, the the moiety comprises polyethylene glycol (PEG). In certain embodiments, the moiety comprises a therapeutic agent. In certain embodiments, the therapeutic agent is a cytotoxic agent. In certain embodiments, the moiety comprises a detectable label. In certain embodiments, the detectable label is an in vivo imaging agent.

In certain embodiments, the present disclosure provides a composition comprising an antibody, or a conjugate, of the present disclosure; and a pharmaceutically acceptable carrier. In certain embodiments, the composition is formulated for parenteral administration. In certain embodiments, the disclosure provides a kit comprising a composition of the present disclosure in a container.

In certain embodiments, the present disclosure provides isolated nucleic acids encoding an scFv of the present disclosure.

In certain embodiments, the present disclosure provides isolated nucleic acids encoding a variable light chain polypeptide, a variable heavy chain polypeptide, or both, of an antibody of the present disclosure.

In certain embodiments, the present disclosure provides an expression vector comprising nucleic acids of the present disclosure and a eukaryotic promoter operably linked to the nucleic acid.

In certain embodiments, the present disclosure provides a non-human host cell comprising the expression vector of the present disclosure. In certain embodiments, the cell expresses the variable light chain polypeptide, the variable heavy chain polypeptide, or both.

In certain embodiments, the present disclosure provides a method of treating a subject having an EphA2-expressing cancer, the method comprising administering to a subject having an EphA2-expressing cancer an antibody, an antibody conjugate, or composition comprising an antibody and/or antibody conjugate of the present disclosure.

In certain embodiments, the present disclosure provides a composition for use in treatment of an EphA2-expressing cancer, wherein the composition an antibody and/or an antibody conjugate of the present disclosure.

The present disclosure x Use of a composition in the treatment of an EphA2-expressing cancer, wherein the composition an antibody and/or an antibody conjugate of the present disclosure.

### BRIEF DESCRIPTION OF THE FIGURES

The scFvs identified by numbers 1-22 in FIGS. 3 and 4 comprise corresponding amino acid sequences identified as scFv1-scFv22 in the sequence listing and the same identification scheme is used for nucleic acid sequences encoding the amino acid sequences of scFv1-scFv22. The nucleic acid sequences further encode N-terminal leader sequences that are cleaved off by mammalian (e.g., human or rodent) cells expressing the encoded scFvs. Where used in FIGS. 1, 3 and 4, “F5” indicates scFv F5. “TS1” indicates scFv TS1. “TS1*” indicates scFv TS1* (note that the VL of TS1* is identical to the VL of TS1 and is indicated as such in FIG. 1), and “A7” indicates scFv D2-1A7.

FIG. 1 shows the results of a Protein A binding assay (see Example 1). scFvs were generated by linking together the VH and VL regions of scFv F5, scFv TS1, scFv TS1*, and D2-1A7. As indicated in the Figure, these hybrid scFvs, along with scFv F5, scFv TS1, scFv TS1*, and D2-1A7, were screened using the Protein A binding assay. F5 scFv, which exhibits desirable Protein A binding properties, was used as a positive control. scFv D1-2A7 was used as a negative control.

FIG. 2, Panels A-D, shows the results of FACS sorting of a set of scFvs designed to contain over 50,000 distinct clones of CDR-H2 variants of scFv TS1. The CDR-H2 variants were generated using targeted mutagenesis. Panel A shows unstained yeast after one round of FACS. Panel B shows stained yeast after one round of FACS, labeled with fluorescently labeled Protein A (x-axis) and Abs against EphA2 (y-axis). The isolated clones were then sorted again. Panel C shows unstained yeast while Panel D shows yeast stained with labeled Abs against the FLAG™ epitope (x-axis) and EphA2 (y-axis).

FIG. 3 shows the results of the Protein A binding assay of EphA2. CDR-H2 variant scFvs were expressed as soluble protein in a mammalian transient system and Protein A binding was measured using the Protein A binding assay. F5 scFv was used as a positive control, and TS1 was used as a negative control.

FIG. 4, Panels A-C show data relating to binding and internalization of Abs on cell lines expressing EphA2. Antibodies were evaluated for the ability to bind and internalize in three cell lines using the CLIA assay (Example 5). F5 scFv, which binds to ErbB2 (and not to EphA2) was used as a negative control, and all data were normalized to the binding and internalization of scFv TS1.

FIG. 5 provides sequences of examples of scFv, scFv linkers, N-terminal peptides, C-terminal tags, C-terminal linkers with tags, C-terminal cysteine-containing sequences, and EphA2 as described herein. The underlining in the amino acid sequences of scFv1-scFv22 indicates the amino acid sequence of the linker (ASTGGGGSGGGGSGGGGSGGGGS (SEQ ID NO: 41), where the amino acid sequence N-terminal to the underlined sequence is the VH amino acid sequence and the amino acid sequence C-terminal to the underlined sequence is the VHL. amino acid sequence.

### DETAILED DESCRIPTION

Provided are new and useful antibodies (e.g, single chain antibodies, e.g., scFvs). Also provided are conjugates, compositions and kits that include the antibodies. Methods of using the Abs, e.g., in the manufacture of immunoliposomes, are also provided.

### Definitions

As used herein, an “antibody” (“Ab”) is an immunoglobulin (“Ig”), or an antigen-binding Ig fragment or other antigen-binding polypeptides that comprise complementary determining regions (“CDRs”) obtained or derived from immunoglobulins (e.g., single chain antibody, e.g., single chain variable fragment (“scFv”)) that exhibits immunospecific antigen binding, i.e., antigen binding mediated by CDRs. Naturally occurring Abs commonly comprise at least four polypeptide chains comprising both at least two heavy chains and at least two light chains, although certain mammalian Abs are known that contain a single (light) chain (e.g., Bence Jones proteins) or that contain one light chain and one heavy chain. Heavy chains can be classified as gamma, mu, alpha, delta, or epsilon, which in turn define the naturally occurring human Ig classes, IgG, IgM, IgA, IgD and IgE, respectively. An example of a typical IgG, IgD or IgE human Ab is one having a structural unit of a tetramer composed of two pairs of polypeptide chains, each pair having one “light” and one “heavy” chain.

“Human antibody” (“human Ab”) as used herein does not require the antibody be produced by any particular method or to require the antibody be naturally occurring. Rather, “human antibody” as used herein refers to an antibody, antigen-binding fragment thereof, or comprising amino acid sequences of a human antibody (e.g., CDRs, frameworks), where the CDRs and/or frameworks do not occur together in nature (i.e., are not naturally occurring).

An “anti-EphA2 Ab” refers to an Ab that immunospecifically binds to EphA2, e.g., the ECD of EphA2. An EphA2-specific Ab does not immunospecifically bind to antigens not present in EphA2 protein.

“CDR” indicates complementarity determining region.

“CLIA” indicates a chelated ligand-induced internalization assay.

“Dil5” indicates the dye 1,1′-Dioctadecyl-3,3,3′,3′-Tetramethylindodicarbocyanine-5,5′-Disulfonic Acid.

“DOD-tri-NTA” indicates dioctadecylamino-tri-NTA compound (compound 12) as described in: Huang, et al, “Facile Synthesis of Multivalent Nitrilotriacetic Acid (NTA) and NTA Conjugates for Analytical and Drug Delivery Applications”, Bioconjugate Chemistry, 2006, vol. 17, p. 1692-1600. NTA indicates nitrilotriacetic acid.

“DSF” indicates differential scanning fluorimetry.

“DSPE” indicates 1,2-distearoyl-sn-glycero-3-phosphoethanolamine.

“ECD” refers to the extracellular domain of a transmembrane protein.

“EphA2” refers to Ephrin type-A receptor 2, also referred to as “epithelial cell kinase (ECK),” a transmembrane protein that is a receptor tyrosine kinase that can bind and be activated by Ephrin-A ligands. The term “EphA2” can refer to any naturally occurring isoforms of EphA2. The amino acid sequence of human EphA2 is recorded as GenBank Accession No. NP_004422.2.

An “epitope” is a site on an antigen, e.g. a site on the EphA2 ECD, to which an Ab immunospecifically binds. Epitopes can be formed both from contiguous amino acids or noncontiguous amino acids juxtaposed by folding (e.g., tertiary folding) of a polypeptide chain forming a protein.

“Fab” indicates an antigen binding fragment of an Ig (regardless of how prepared) including variable domain and first constant domain.

“FACS” indicates fluorescence activated cell sorting.

“His6” indicates a hexahistidine peptide.

“HSPC” indicates hydrogenated soy phosphatidylcholine.

“Ig” indicates an immunoglobulin. An Ig light or heavy chain variable region is composed of a plurality of “framework” regions (FR) alternating with three hypervariable regions, also called “complementarity determining regions” or “CDRs”. The extent of the framework regions and CDRs can be defined based on homology to sequences found in public databases. See, for example, “Sequences of Proteins of Immunological Interest,” E. Kabat et al., Sequences of proteins of immunological interest, 4th ed. U.S. Dept. Health and Human Services, Public Health Services, Bethesda, Md. (1987). All Ab sequence numbering used herein is as defined by Kabat et al.

“IgG” indicates an immunoglobulin of the G isotype.

“Isolated” refers to an entity of interest (e.g., a protein, e.g., an Ab) that is in an environment different from that in which the entity may naturally occur or in which it is manufactured or synthesized. An “isolated” entity is separated from all or some of the components that accompany it in the cell or reaction vessel in which it is made. A subject Ab can be substantially pure. “Substantially pure” can refer to compositions in which at least 75%, at least 85%, at least 90%, at least 95%, at least 98%, at least 99%, or more than 99%, of the total composition is the entity of interest (e.g., an anti-EphA2 Ab disclosed herein).

“Mal-PEG-DSPE” indicates maleimido-PEG(2000)-DSPE (1,2-distearoyl-sn-glycero-3-phosphoethanolam ine-N-[maleimide(polyethylene glycol)-2000].

“MFI” indicates median fluorescence intensity.

“NTA(Ni)” indicates nickel-nitrilotriacetic acid.

“PBS” indicates phosphate buffered saline.

“PEG” indicates polyethylene glycol.

“PEG-DSPE” indicates methoxy-PEG(2000)-distearoylphosphatidylethanolamine.

“scFv” indicates a single chain Fv, which is a protein comprised of a single polypeptide chain in which a VH and a VL are covalently linked to each other, typically via a linker peptide that allows the formation of a functional antigen binding site comprised of VH and VL CDRs.

“SEC” indicates size exclusion chromatography.

“SM” indicates sphingomyelin.

“Tm” indicates melting temperature.

“TS1” indicates scFv-TS1 as disclosed below.

“TS1*” indicates scFv-TS1* as disclosed below.

“VH” indicates the variable region of an Ig heavy chain, which may also referred to as a VH polypeptide.

“VL” indicates the variable region of an Ig light chain, which may also be referred to as a VL polypeptide.

### Antibodies

Provided by the present disclosure are anti-EphA2 Abs that include framework sequences and CDR sequences that confer useful properties upon the Abs. While the Abs set forth in the Examples are scFvs, the CDR and human framework sequences disclosed herein can be readily and beneficially incorporated into IgA, IgD, IgE, IgG, and IgM Abs. In certain embodiments, such properties include stability (e.g., thermostability, e.g., a melting temperature of greater than 65° C., greater than or equal to 67° C., or at least about 70° C.), ≧30%, ≧40%, or ≧50% binding to Protein A, or both.

Framework Regions

In certain embodiments, an scFv disclosed herein includes one or any combination of VH FR1, VH FR2, VH FR3, VL FR1, VL FR2, and VL FR3, wherein:

the VH FR1 is a human VH FR1 comprising the amino acid sequence QVQLVX¹SGGGLVQPGGSLRLSCAASGFTFS (SEQ ID NO: 1), wherein X¹ is an amino acid other than E, and in certain embodiments X¹ is Q;

the VH FR2 is a human VH FR2 comprising the amino acid sequence WVRQAPGKGLEWVX² (SEQ ID NO: 2), wherein X² is an amino acid other than S, and in certain embodiments X² is A;

the VL FR1 is a human VL FR1 comprising the amino acid sequence X³SX⁴LTQPPSVSX⁵APGQX⁶VTIX⁷C (SEQ ID NO: 3), wherein: X³ is an amino acid other than Q, X⁴ is an amino acid other than V, X⁵ is an amino acid other than G, X⁶ is an amino acid other than R, and X⁷ is an amino acid other than S, and in certain embodiments X⁴ is V, X⁵ is G, and X⁶ is R;

in certain embodiments, the VL FR1 is a human VL FR1 comprising the amino acid sequence X⁸SVLTQPPSVSGAPGQRVTIX⁹C (SEQ ID NO: 4), wherein X⁸ is an amino acid other than Q, and wherein X⁹ is an amino acid other than S, and in certain embodiments X⁸ is S and/or X⁹ is T;

the VL FR2 is a human VL FR2 comprising the amino acid sequence WYQQX¹⁰PGTAPKLLIY (SEQ ID NO: 5), wherein X¹⁰ is an amino acid other than L, and/or in certain embodiments X¹⁰ is K;

and

the VL FR3 is a human VL FR3 comprising the amino acid sequence GVPDRFSGX¹¹X¹²SGTSASLX¹³ITGX¹⁴QAEDEADYYC (SEQ ID NO: 6), wherein: X¹¹ is an amino acid other than F, X¹² is an amino acid other than K, X¹³ is an amino acid other than A, or X¹⁴ is an amino acid other than L, and in certain embodiments X¹¹ is S, X¹² is S, X¹³ is T, and/or X¹⁴ is A;

in certain embodiments the VL FR3 is a human VL FR3 comprising the amino acid sequence GVPDRFSGX¹⁵X¹⁶SGTSASLAITGLQAEDEADYYC (SEQ ID NO: 7), wherein: X¹⁵ is an amino acid other than F, or X¹⁶ is an amino acid other than K, and in certain embodiments X¹⁵ is S and/or X¹⁶ is G.

In certain embodiments, an Ab disclosed herein includes one or any combination of VH FR1, VH FR2, VH FR3, VL FR1, VL FR2, and VL FR3 set forth in Table 1. In one embodiment, the Ab is an scFv and contains all of the frameworks of Table 1.

TABLE 1
Examples of Framework Sequences

| | |
|---|---|
| VH FR1 (SEQ ID | QVQLVQSGGGLVQPGGSLRLSCAASGFTFS |
| NO: 8) | |
| VH FR2 (SEQ ID | WVRQAPGKGLEWVA |
| NO: 9) | |
| VH FR2 (SEQ ID | WVRQAPGKGLEWVT |
| NO: 10) | |
| VH FR3 (SEQ ID | RFTISRDNSKNTLYLQMNSLRAEDTAVYYCAR |
| NO: 11) | |
| VH FR4 (SEQ ID | WGQGTLVTVSS |
| NO: 12) | |
| VL FR1 (SEQ ID | SSELTQPPSVSVAPGQTVTITC |
| NO: 13) | |
| VL FR2 (SEQ ID | WYQQKPGTAPKLLIY |
| NO: 14) | |
| VL FR3 (SEQ ID | GVPDRFSGSSSGTSASLTITGAQAEDEADYYC |
| NO: 15) | |
| VL FR4 (SEQ ID | FGGGTKLTVLG |
| NO: 16) | |

In certain aspects, an Ab (e.g., an scFv) disclosed herein is thermostable, e.g., such that the Ab is well-suited for robust and scalable manufacturing. As used herein, a “thermostable” Ab is an Ab having a melting temperature (Tₘ) of 70° C. or higher, e.g., as measured using differential scanning fluorimetry (DSF). Example Abs having suitable melting temperatures are described in the Experimental section below.

In certain embodiments, an Ab disclosed herein exhibits 30%, 40%, or beneficially 50% or greater binding to Protein A, e.g., as determined using the assay described in Example 1.

EphA2 Binding

Antibodies of particular interest include anti-EphA2 Abs that bind to the extracellular domain of EphA2 polypeptide, i.e., the part of the EphA2 protein spanning at least amino acid residues 25 to 534 of the sequence set forth in GenBank Accession No. NP_004422.2 or UniProt Accession No. P29317.

In certain embodiments, an anti-EphA2 antibodies (e.g., scFv) disclosed herein includes a VH CDR1, VH CDR2, VH CDR3, VL CDR1, VL CDR2, and VL CDR3 each with a sequence as set forth in Table 2A. In other embodiments the VH CDR2 sequence (also referred to as CDRH2) will be any one selected from the 18 different VH CDR2 sequences set forth in Table 2B.

TABLE 2A
Complementary Determining Regions (CDRs)

| | |
|---|---|
| VH CDR1 (SEQ ID NO: 17) | SYAMH |
| VH CDR2 (SEQ ID NO: 18) | X¹⁷ISPX¹⁸GX¹⁹NX²⁰YYADSVKG |
| VH CDR3 (SEQ ID NO: 19) | ASVGATGPFDI |
| VL CDR1 (SEQ ID NO: 20) | QGDSLRSYYAS |
| VL CDR2 (SEQ ID NO: 21) | GENNRPS |
| VL CDR3 (SEQ ID NO: 22) | NSRDSSGTHLTV |

For the VH CDR2 in Tables 2A and 3: X¹⁷ is A or V; X¹⁸ is A, D, H, N, P, S, or T; X¹⁹ is A, R, H or P; and X²⁰ is K or T. In certain embodiments: X¹⁷ is A; X¹⁸ is H or P; or X¹⁹ is R or P. In certain embodiments: X¹⁷ is V; X¹⁸ is H or P; X¹⁹ is R or N; and X²⁰ is K or T. In certain embodiments: X¹⁷ is V; X¹⁸ is H or D; X¹⁹ is H or N; and X²⁰ is T.

Eighteen particular examples of suitable VH CDR2 sequences are set forth in Table 2B:

TABLE 2B
CDRH2 Sequences

| | |
|---|---|
| VH CDR2 (SEQ ID NO: 23) | VISPAGNNTYYADSVKG |
| VH CDR2 (SEQ ID NO: 24) | VISPAGRNKYYADSVKG |
| VH CDR2 (SEQ ID NO: 25) | VISPDGHNTYYADSVKG |
| VH CDR2 (SEQ ID NO: 26) | VISPHGRNKYYADSVKG |
| VH CDR2 (SEQ ID NO: 27) | VISRRGDNKYYADSVKG |
| VH CDR2 (SEQ ID NO: 28) | VISNNGHNKYYADSVKG |
| VH CDR2 (SEQ ID NO: 29) | VISPAGPNTYYADSVKG |
| VH CDR2 (SEQ ID NO: 30) | VISPSGHNTYYADSVKG |
| VH CDR2 (SEQ ID NO: 31) | VISPNGHNTYYADSVKG |
| VH CDR2 (SEQ ID NO: 32) | AISPPGHNTYYADSVKG |
| VH CDR2 (SEQ ID NO: 33) | VISPTGANTYYADSVKG |
| VH CDR2 (SEQ ID NO: 34) | VISPHGSNKYYADSVKG |
| VH CDR2 (SEQ ID NO: 35) | VISNNGHNTYYADSVKG |
| VH CDR2 (SEQ ID NO: 36) | VISPAGTNTYYADSVKG |
| VH CDR2 (SEQ ID NO: 37) | VISPPGHNTYYADSVKG |
| VH CDR2 (SEQ ID NO: 38) | VISHDGTNTYYADSVKG |
| VH CDR2 (SEQ ID NO: 39) | VISRHGNNKYYADSVKG |
| VH CDR2 (SEQ ID NO: 40) | VISYDGSNKYYADSVKG |

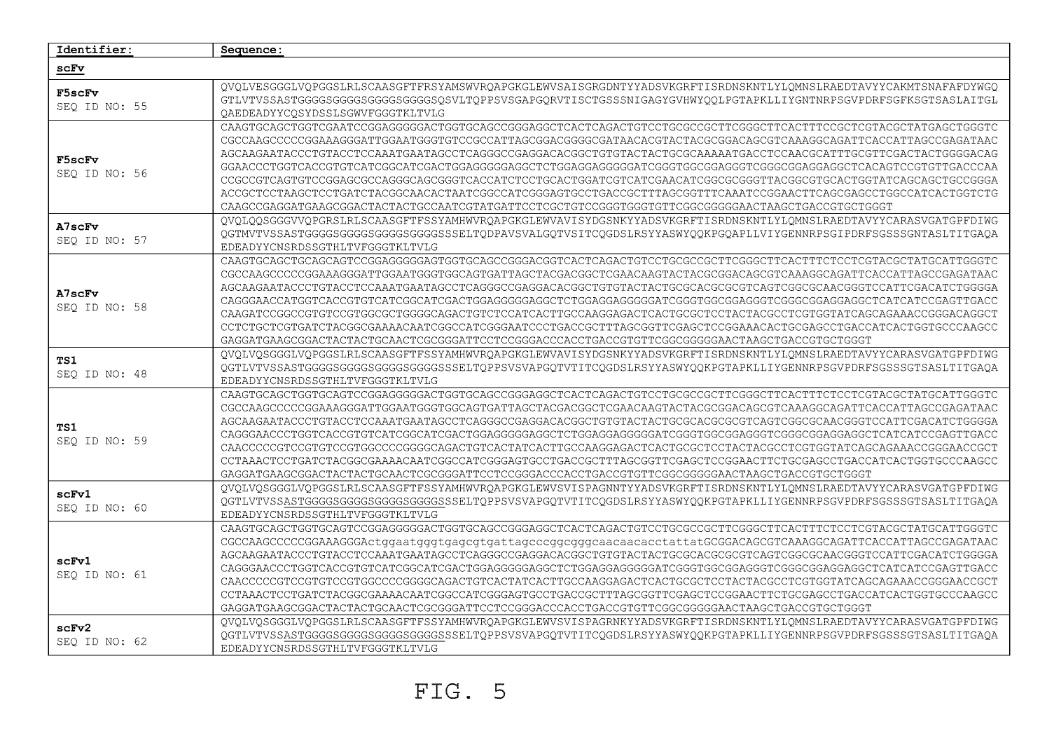

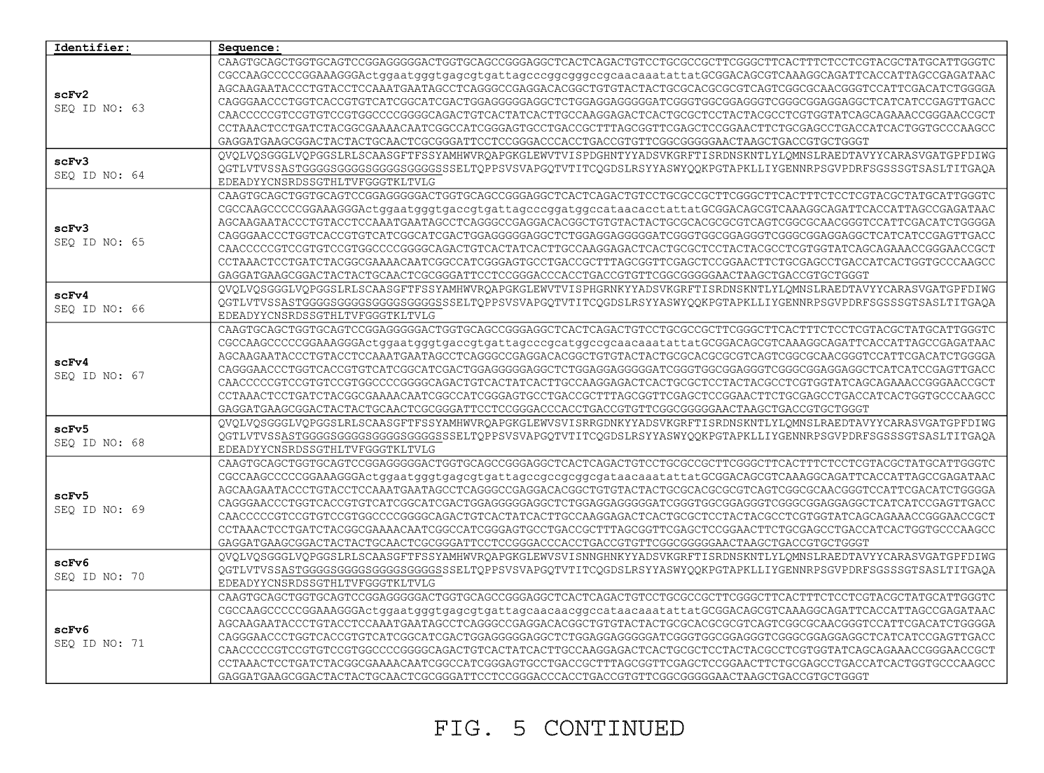

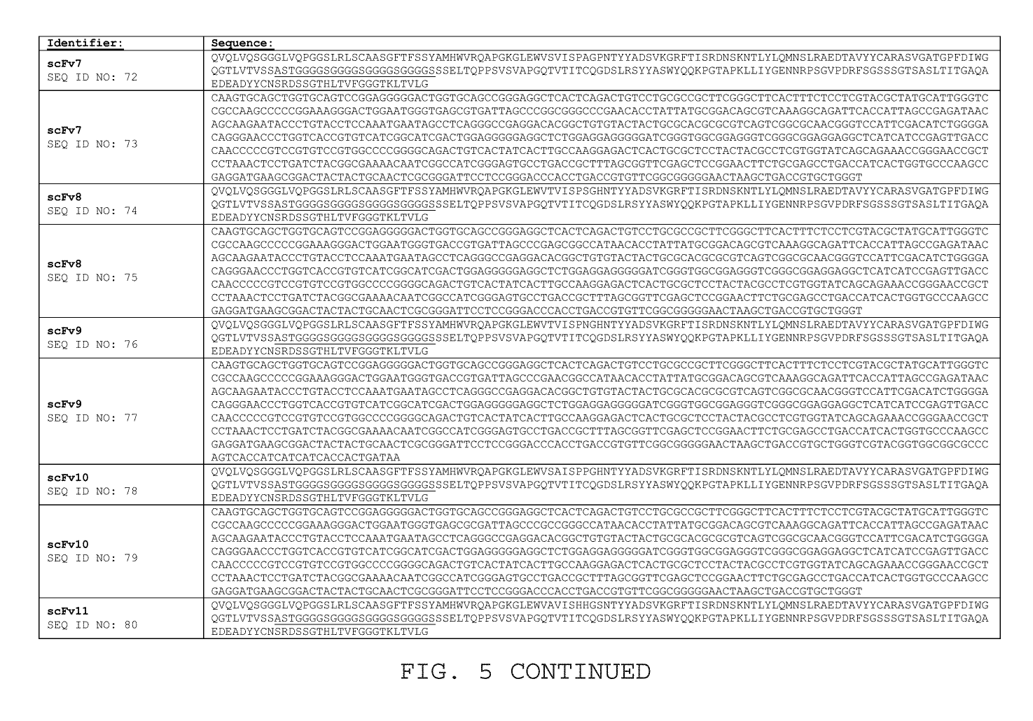

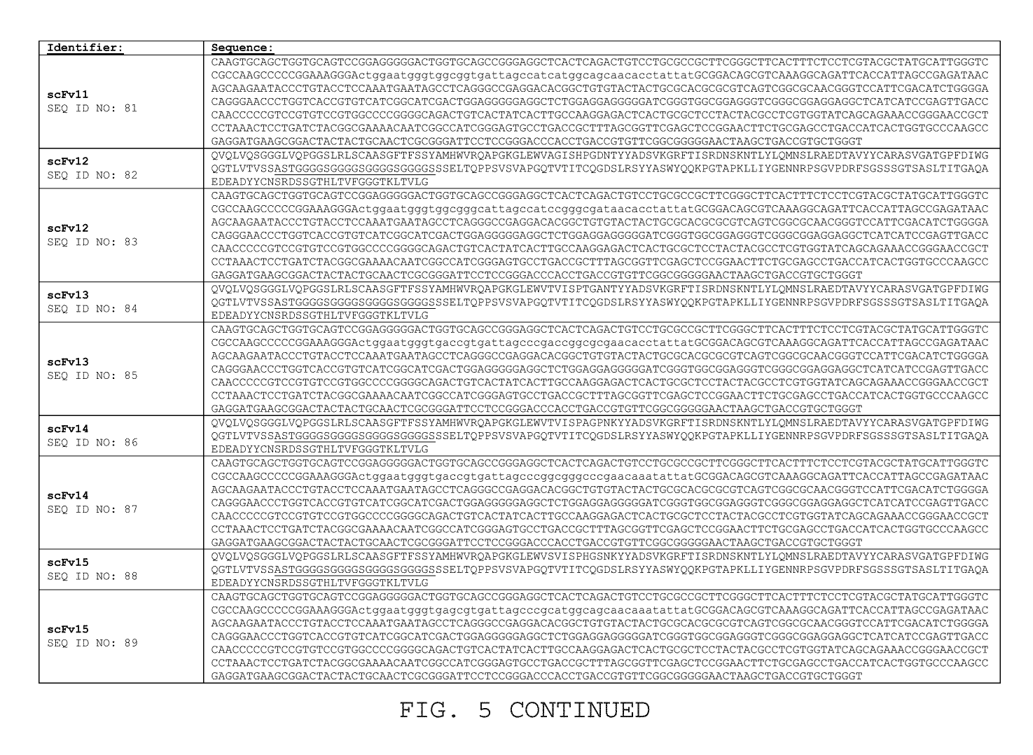

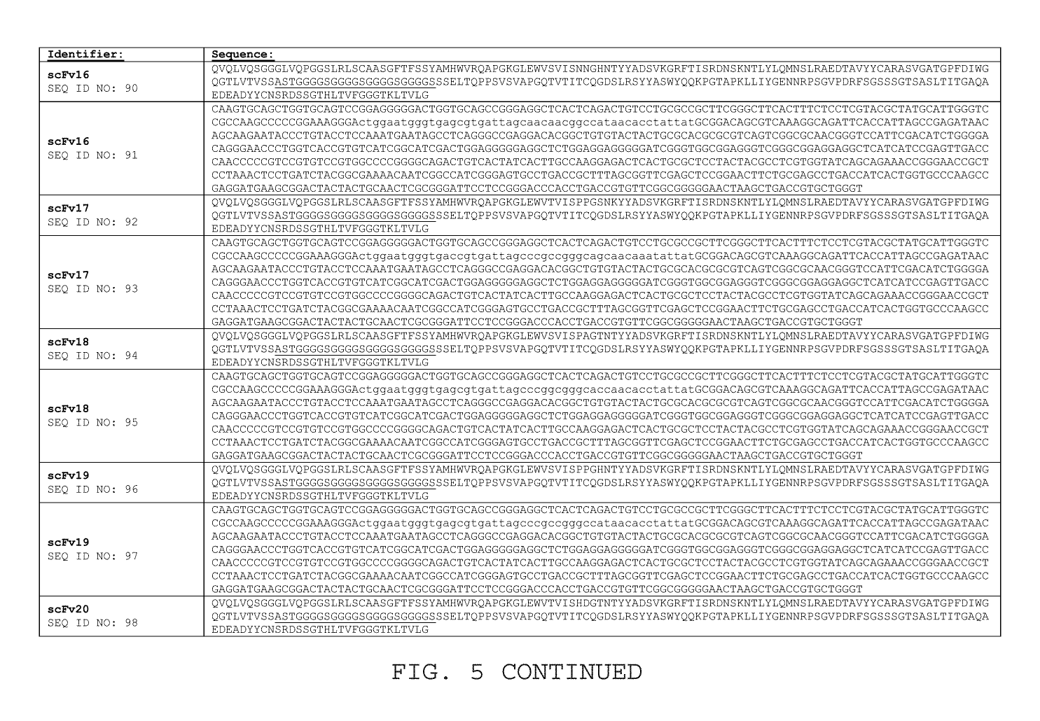

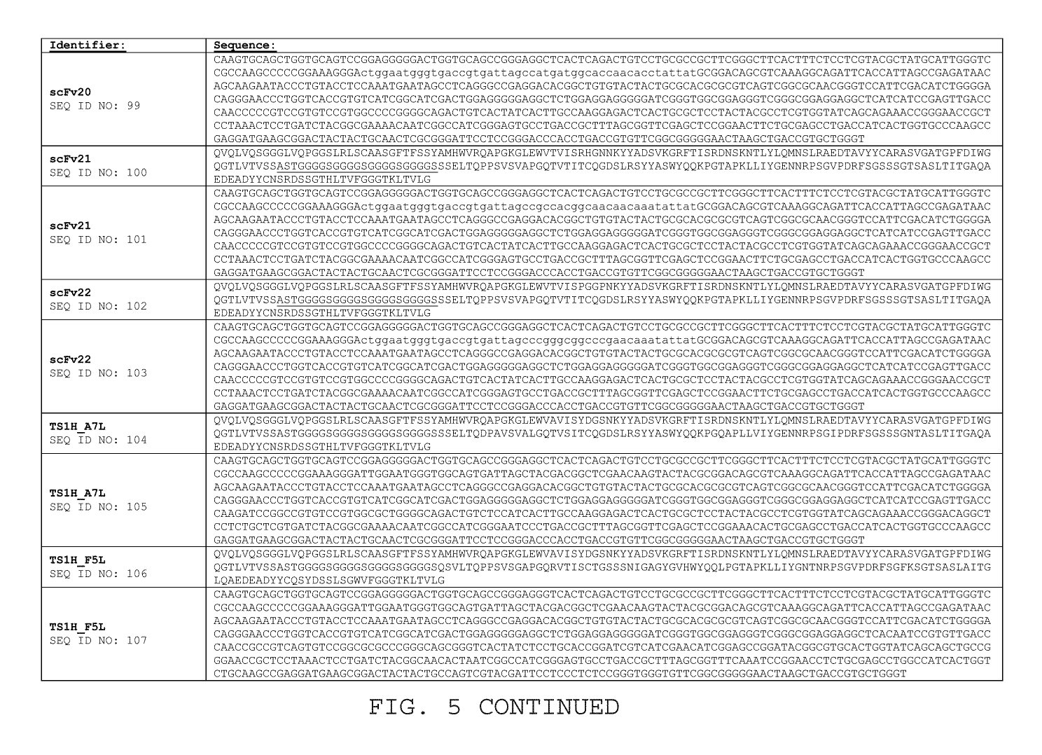

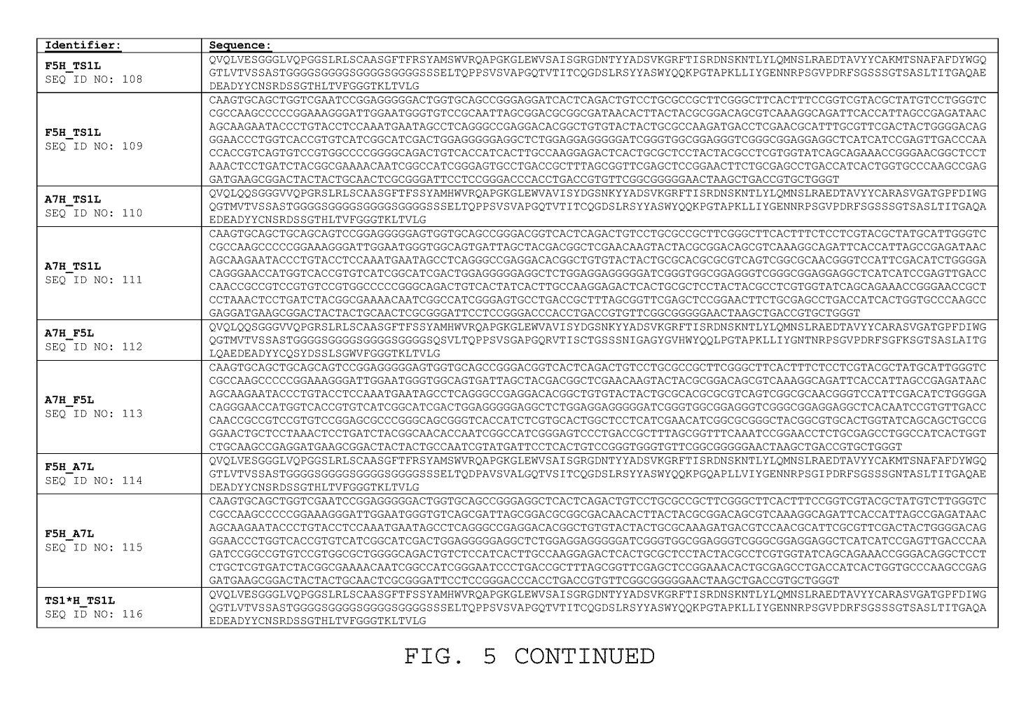

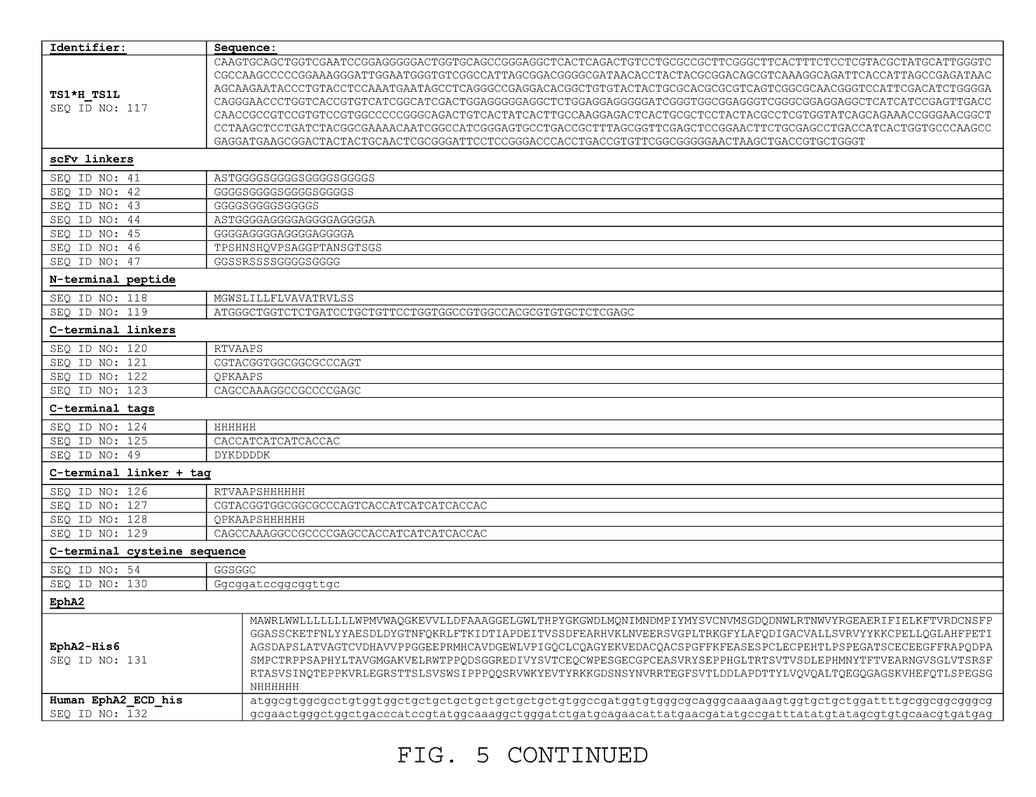

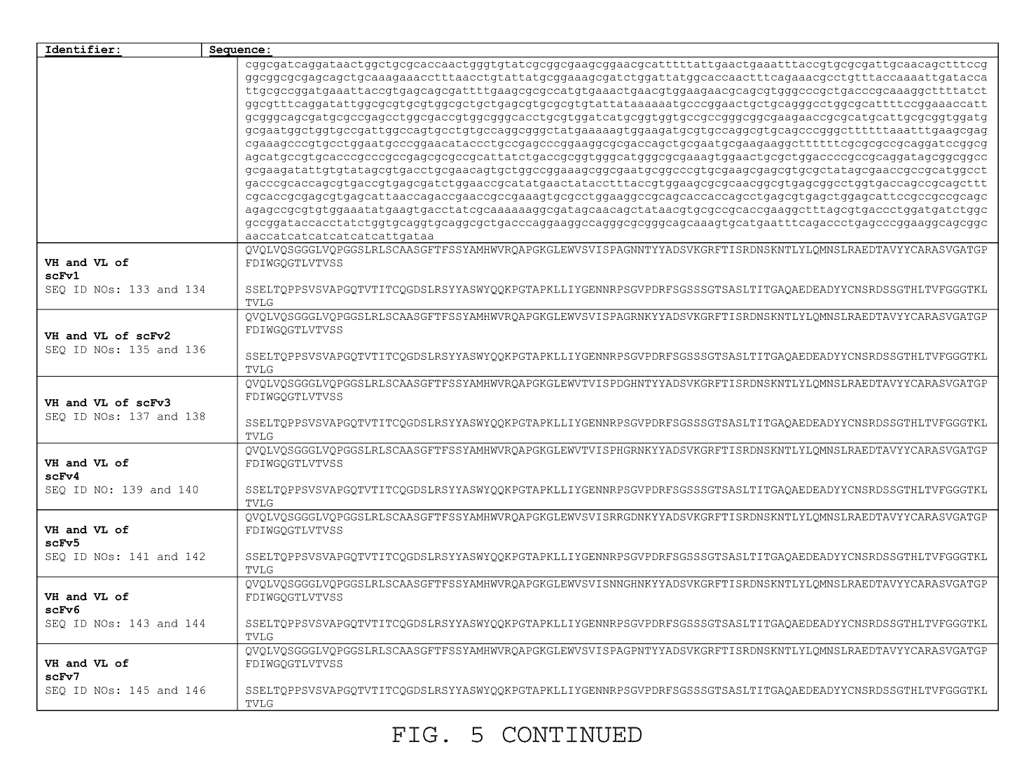

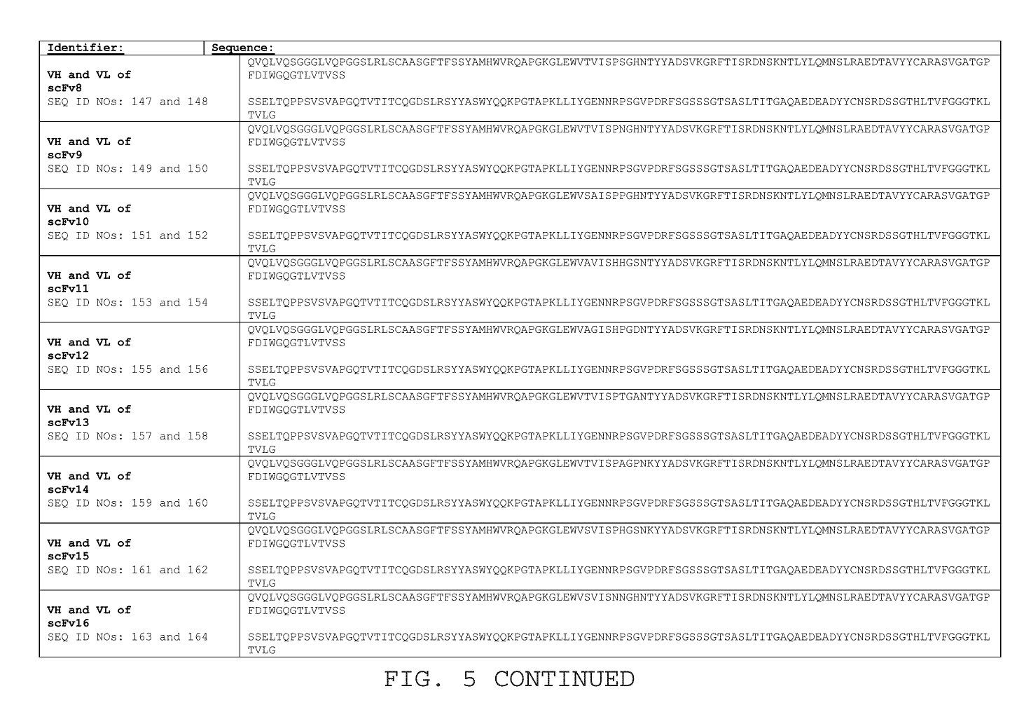

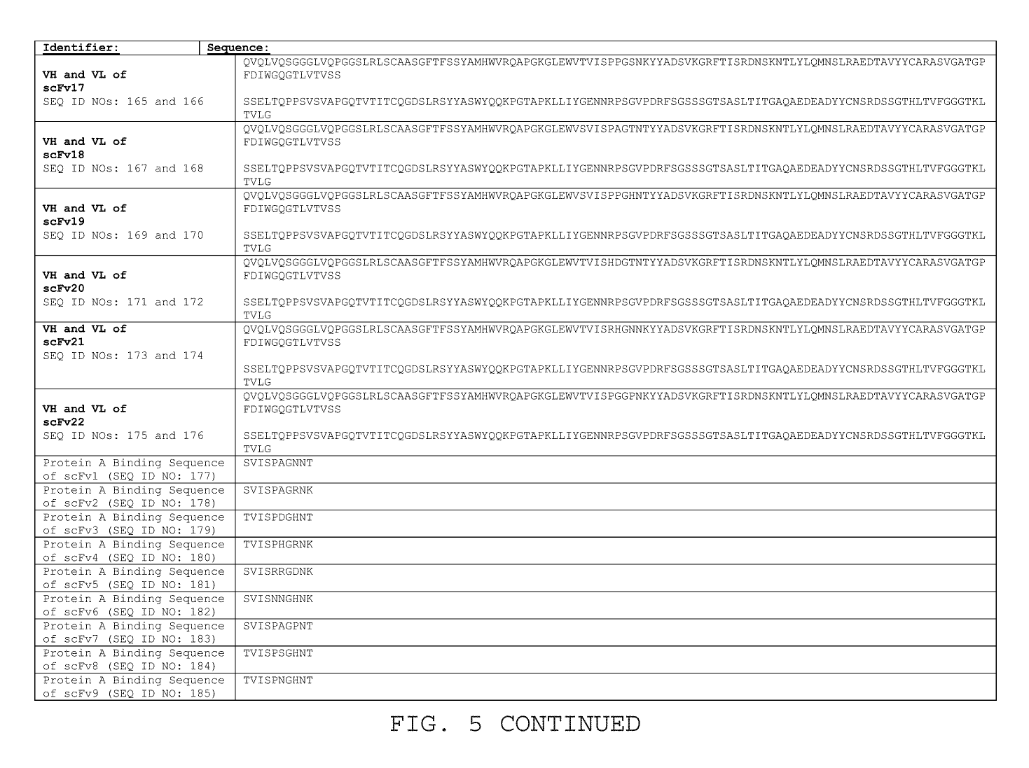

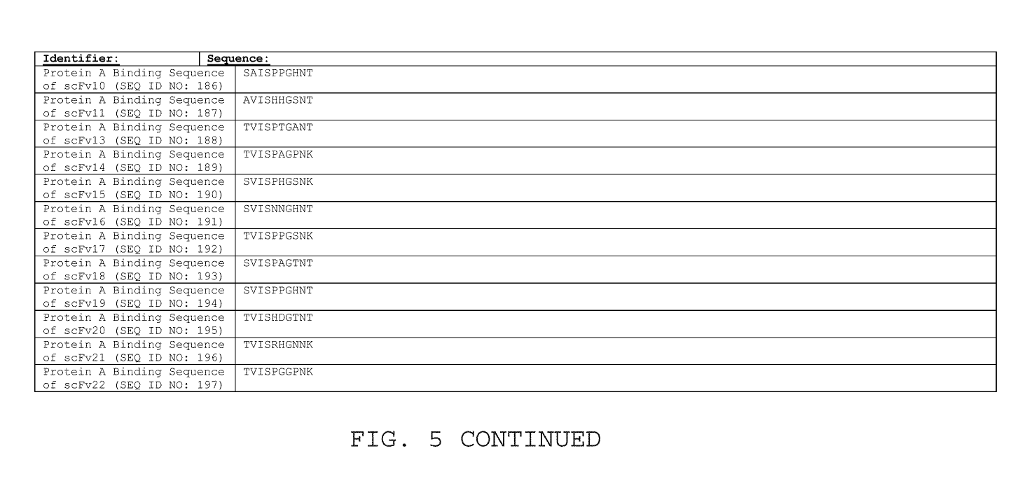

Examples of scFvs provided by the present disclosure are provided in FIG. 5. FIG. 5 provides the amino acid sequences of scFv1-scFv22, as well as, the corresponding encoding nucleic acid sequences. The underlining in the amino acid sequence of each of scFv2-scFv22 indicates the amino acid sequence of the linker (ASTGGGGSGGGGSGGGGSGGGGS (SEQ ID NO: 41). The amino acid sequence N-terminal to the underlined amino acid sequence of scFv1-scFv22 is the amino acid sequence of the VH polypeptide of the scFv. The amino acid sequence C-terminal to the underlined amino acid sequence of scFv1-scFv22 is the amino acid sequence of the VL polypeptide of the scFv.

Accordingly, the present disclosure provides antibodies (including scFvs) having a VH and VL, wherein the VH has an amino acid sequence at least 95% identical to a VH of any one of scFv1 to scFv22 as set out in FIG. 5. The present disclosure also provides antibodies (including scFvs) comprising a VH and a VL, wherein the VH comprises an amino acid sequence having at least 95% amino acid sequence identity to a VH and a VL of the VH and VL of any one of scFv1 to scFv22 as set out in FIG. 5. The present disclosure also provides antibodies (such as an scFv) having a VH and a VL, wherein each of the VH and VL comprises an amino acid sequence of the VH and VL of a VH and VL of an scFv of FIG. 5.

The amino acid sequences of the VH and VL of scFv1 to scFv22 of FIG. 5 are as follows: VH and VL of scFv1 (SEQ ID NOs: 133 and 134, respectively), VH and VL of scFv2 (SEQ ID NOs: 135 and 136, respectively), VH and VL of scFv3 (SEQ ID NOs: 137 and 138, respectively), VH and VL of scFv4 (SEQ ID NO: 139 and 140, respectively), VH and VL of scFv5 (SEQ ID NOs: 141 and 142, respectively), VH and VL of scFv6 (SEQ ID NOs: 143 and 144, respectively), VH and VL of scFv7 (SEQ ID NOs: 145 and 146, respectively), VH and VL of scFv8 (SEQ ID NOs: 147 and 148, respectively), VH and VL of scFv9 (SEQ ID NOs: 149 and 150, respectively), VH and VL of scFv10 (SEQ ID NOs: 151 and 152, respectively), VH and VL of scFv1 1 (SEQ ID NOs: 153 and 154, respectively), VH and VL of scFv12 (SEQ ID NOs: 155 and 156, respectively), VH and VL of scFv13 (SEQ ID NOs: 157 and 158, respectively), VH and VL of scFv14 (SEQ ID NOs: 159 and 160, respectively), VH and VL of scFv15 (SEQ ID NOs: 161 and 162, respectively), VH and VL of scFv16 (SEQ ID NOs: 163 and 164, respectively), VH and VL of scFv17 (SEQ ID NOs: 165 and 166, respectively), VH and VL of scFv18 (SEQ ID NOs: 167 and 168, respectively), VH and VL of scFv19 (SEQ ID NOs: 169 and 170, respectively), VH and VL of scFv20 (SEQ ID NOs: 171 and 172, respectively), VH and VL of scFv21 (SEQ ID NOs: 173 and 174, respectively), or VH and VL of scFv22 (SEQ ID NOs: 175 and 176, respectively).

The present disclosure also provides scFvs having at least 95% amino acid sequence identity to the amino acid sequence of SEQ ID NO: 60 [scFv1], SEQ ID NO: 62 [scFv2], SEQ ID NO: 64 [scFv3] SEQ ID NO:66 [scFv4], SEQ ID NO: 68 [scFv5], SEQ ID NO: 70 [scFv6], SEQ ID NO: 72 [scFv7]. SEQ ID NO: 74 [scFv8], SEQ ID NO: 76 [scFv9], SEQ ID NO: 78 [scFv10], SEQ ID NO: 80 [scFv1 1], SEQ ID NO: 82 [scFv12], SEQ ID NO: 84 [scFv13], SEQ ID NO: 86 [scFv14], SEQ ID NO: 88 [scFv15], SEQ ID NO: 90 [scFv16], SEQ ID NO: 92 [scFv17], SEQ ID NO: 94 [scFv18], SEQ ID NO: 96 [scFv19], SEQ ID NO: 98 [scFv20], SEQ ID NO: 100 [scFv21], or SEQ ID NO: 102 [scFv22], as well as nucleic acids encoding same. The present disclosure also provides scFvs having the amino acid sequence of SEQ ID NO: 60 [scFv1], SEQ ID NO: 62 [scFv2], SEQ ID NO: 64 [scFv3] SEQ ID NO:66 [scFv4], SEQ ID NO: 68 [scFv5], SEQ ID NO: 70 [scFv6], SEQ ID NO: 72 [scFv7]. SEQ ID NO: 74 [scFv8], SEQ ID NO: 76 [scFv9], SEQ ID NO: 78 [scFv10], SEQ ID NO: 80 [scFv1 1], SEQ ID NO: 82 [scFv12], SEQ ID NO: 84 [scFv13], SEQ ID NO: 86 [scFv14], SEQ ID NO: 88 [scFv15], SEQ ID NO: 90 [scFv16], SEQ ID NO: 92 [scFv17], SEQ ID NO: 94 [scFv18], SEQ ID NO: 96 [scFv19], SEQ ID NO: 98 [scFv20], SEQ ID NO: 100 [scFv21], or SEQ ID NO: 102 [scFv22], as well as nucleic acids encoding same.

The present disclosure also provides polypeptides that bind Protein A, which polypeptides may be present in an antibody (e.g., an scFv), and which comprise the amino acid sequence:

X^{a}X¹⁷ISX^{b}X¹⁸GX¹⁹NX²⁰ (SEQ ID NO: 198)

wherein

X^{a} is S, T or A;

X¹⁷ is A, or V;

X^{b} is P, R, N or H,

X¹⁸ and X¹⁹ are any amino acid; and

X²⁰ is K or T.

In one embodiment, the polypeptide (which may be present in an antibody (e.g., an scFv)) comprises the amino acid sequence of SEQ ID NO: 198, wherein X¹⁸ is A, D, H, N, P, S or T; and X¹⁹ is A, R, H, N or P.

In one embodiment, such polypeptides comprise, from N- to C-terminus,

a VH CDR1 comprising an amino acid sequence of SYAMH (SEQ ID NO:17);

a Protein A binding sequence of the formula of SEQ ID NO: 198, wherein X¹⁸ is any amino acid, or is A, D, H, N, P, S or T; and wherein X¹⁹ is any amino acid or is A, R, H, N or P; and

a VH CDR3 comprising an amino acid sequence of ASVGATGPFDI (SEQ ID NO: 19).

Where the polypeptide of SEQ ID NO:198 is present in an antibody (e.g., an scFv), the antibody may comprise a VL CDR1 comprising the amino acid sequence QGDSLRSYYAS (SEQ ID NO: 20), a VL CDR2 comprising the amino acid sequence GENNRPS (SEQ ID NO: 21) and a VL CDR3 comprising the amino acid sequence NSRDSSGTHLTV (SEQ ID NO: 22). In one embodiment where the polypeptide is an antibody (e.g., scFv), the antibody (e.g., scFv) includes one or both of a VH FR1 and VH FR2, wherein the VH FR1 comprises the amino acid sequence of QVQLVX¹SGGGLVQPGGSLRLSCAASGFTFS (SEQ ID NO: 1), wherein X¹ is an amino acid other than E, and in certain embodiments X¹ is Q; the VH FR2 is a comprises the amino acid sequence WVVVRQAPGKGLEWV (SEQ ID NO: 202). Such antibodies (e.g., scFv) may also include one or more of a VL FR1 comprising the amino acid sequence of SEQ ID NO:3, a VL FR2 comprising the amino acid sequence of SEQ ID NO: 4, and a VL FR3 comprising an amino acid sequence of SEQ ID NO:5, and a VL FR3 comprising the amino acid sequence of SEQ ID NO:6. In one embodiment such antibodies (e.g., scFv) comprise one or more of any combination of a VH FR1, VH FR2, VH FR3, VL FR1, VL FR2, and VL FR3 of Table 1.

The present disclosure also provides polypeptides that bind Protein A, which polypeptides may be present in an antibody (e.g., an scFv), and comprise the amino acid sequence:

X^{a}VISX^{b}X¹⁸GX¹⁹NX²⁰ (SEQ ID NO: 199),

wherein

X^{a} is S or T,

X^{b} is P, R, or N,

X¹⁸ is any amino acid;

X¹⁹ is H, N, R, T, A, D, P, or S; and

X²⁰ is K or T

In one embodiment, the polypeptide (which may be present in an antibody (e.g., an scFv) comprises the amino acid sequence of SEQ ID NO: 199, wherein X¹⁸ is A, D, H, N, P, S or T.

In one embodiment, the polypeptide (which may be present in an antibody (e.g., an scFv) comprises the amino acid sequence of SEQ ID NO: 199, wherein X¹⁸ is A, D, H, N, P, S or T; and X¹⁹ is A, R, H, N or P.

In one embodiment, such polypeptides comprise, from N- to C-terminus,

a VH CDR1 comprising an amino acid sequence of SYAMH (SEQ ID NO:17);

a Protein A binding sequence of the formula of SEQ ID NO: 199, wherein X¹⁸ is any amino acid, or is A, D, H, N, P, S or T; and wherein X¹⁹ is any amino acid or is A, R, H, N or P; and

a VH CDR3 comprising an amino acid sequence of ASVGATGPFDI (SEQ ID NO: 19).

Where the polypeptide of SEQ ID NO:199 is present in an antibody (e.g., an scFv), the antibody may comprise a VL CDR1 comprising the amino acid sequence QGDSLRSYYAS (SEQ ID NO: 20), a VL CDR2 comprising the amino acid sequence GENNRPS (SEQ ID NO: 21) and a VL CDR3 comprising the amino acid sequence NSRDSSGTHLTV (SEQ ID NO: 22). In one embodiment where the polypeptide is an antibody (e.g., scFv), the antibody (e.g., scFv) includes one or both of a VH FR1 and VH FR2, wherein the VH FR1 comprises the amino acid sequence of QVQLVX¹SGGGLVQPGGSLRLSCAASGFTFS (SEQ ID NO: 1), wherein X¹ is an amino acid other than E, and in certain embodiments X¹ is Q; the VH FR2 is a comprises the amino acid sequence WVVVRQAPGKGLEWV (SEQ ID NO: 202). Such antibodies (e.g., scFv) may also include one or more of a VL FR1 comprising the amino acid sequence of SEQ ID NO:3, a VL FR2 comprising the amino acid sequence of SEQ ID NO: 4, and a VL FR3 comprising an amino acid sequence of SEQ ID NO:5, and a VL FR3 comprising the amino acid sequence of SEQ ID NO:6. In one embodiment such antibodies (e.g., scFv) comprise one or more of any combination of a VH FR1, VH FR2, VH FR3, VL FR1, VL FR2, and VL FR3 of Table 1.

The present disclosure also provides polypeptides, which polypeptides may be present in an antibody (e.g., an scFv), that bind Protein A, and comprise the amino acid sequence:

X^{a}VISX^{b}X¹⁸GX¹⁹NX²⁰ (SEQ ID NO: 200)

wherein

X^{a} is S or T,

X^{b} is P, R, or N,

X¹⁸ is any amino acid;

X¹⁹ is H, N, R, or T; and

X²⁰ is T or K.

In one embodiment, the polypeptide (which may be present in an antibody (e.g., an scFv) comprises the amino acid sequence of SEQ ID NO: 200, wherein X¹⁸ is A, D, H, N, P, S or T. In one embodiment, the polypeptide (which may be present in an antibody (e.g., an scFv) comprises the amino acid sequence of SEQ ID NO: 200, wherein X¹⁸ is A, D, H, N, P, S or T; and X¹⁹ is A, R, H, N or P.

In one embodiment, such polypeptides comprise, from N- to C-terminus,

a VH CDR1 comprising an amino acid sequence of SYAMH (SEQ ID NO:17);

a Protein A binding sequence of the formula of SEQ ID NO: 200, wherein X¹⁸ is any amino acid, or is A, D, H, N, P, S or T; and wherein X¹⁹ is any amino acid or is A, R, H, N or P; and

a VH CDR3 comprising an amino acid sequence of ASVGATGPFDI (SEQ ID NO: 19).

Where the polypeptide of SEQ ID NO:200 is present in an antibody (e.g., an scFv), the antibody may comprise a VL CDR1 comprising the amino acid sequence QGDSLRSYYAS (SEQ ID NO: 20), a VL CDR2 comprising the amino acid sequence GENNRPS (SEQ ID NO: 21) and a VL CDR3 comprising the amino acid sequence NSRDSSGTHLTV (SEQ ID NO: 22). In one embodiment where the polypeptide is an antibody (e.g., scFv), the antibody (e.g., scFv) includes one or both of a VH FR1 and VH FR2, wherein the VH FR1 comprises the amino acid sequence of QVQLVX¹SGGGLVQPGGSLRLSCAASGFTFS (SEQ ID NO: 1), wherein X¹ is an amino acid other than E, and in certain embodiments X¹ is Q; the VH FR2 is a comprises the amino acid sequence WVRQAPGKGLEWV (SEQ ID NO: 202). Such antibodies (e.g., scFv) may also include one or more of a VL FR1 comprising the amino acid sequence of SEQ ID NO:3, a VL FR2 comprising the amino acid sequence of SEQ ID NO: 4, and a VL FR3 comprising an amino acid sequence of SEQ ID NO:5, and a VL FR3 comprising the amino acid sequence of SEQ ID NO:6. In one embodiment such antibodies (e.g., scFv) comprise one or more of any combination of a VH FR1, VH FR2, VH FR3, VL FR1, VL FR2, and VL FR3 of Table 1.

The present disclosure also provides polypeptides, which polypeptides may be present in an antibody (e.g., an scFv), that bind Protein A, and comprise the amino acid sequence:

X^{a}X¹⁷ISX^{b}X¹⁸GX¹⁹NX²⁰ (SEQ ID NO: 201),

wherein

X^{a} is S or T,

X¹⁷ is V;

X^{b} is P,

X¹⁸ is any amino acid;

X¹⁹ is H, N, R, or T; and

X²⁰ is T or K.

In one embodiment, the polypeptide (which may be present in an antibody (e.g., an scFv) comprises the amino acid sequence of SEQ ID NO: 201, wherein X¹⁸ is A, D, H, N, P, S or T.

In one embodiment, the polypeptide (which may be present in an antibody (e.g., an scFv) comprises the amino acid sequence of SEQ ID NO: 201, wherein X¹⁸ is A, D, H, N, P, S or T; and X¹⁹ is A, R, H, N or P.

In one embodiment, such polypeptides comprise, from N- to C-terminus,

a VH CDR1 comprising an amino acid sequence of SYAMH (SEQ ID NO:17);

a Protein A binding sequence of the formula of SEQ ID NO: 201, wherein X¹⁸ is any amino acid, or is A, D, H, N, P, S or T; and wherein X¹⁹ is any amino acid or is A, R, H, N or P; and

a VH CDR3 comprising an amino acid sequence of ASVGATGPFDI (SEQ ID NO: 19).

Where the polypeptide of SEQ ID NO: 201 is present in an antibody (e.g., an scFv), the antibody may comprise a VL CDR1 comprising the amino acid sequence QGDSLRSYYAS (SEQ ID NO: 20), a VL CDR2 comprising the amino acid sequence GENNRPS (SEQ ID NO: 21) and a VL CDR3 comprising the amino acid sequence NSRDSSGTHLTV (SEQ ID NO: 22). In one embodiment where the polypeptide is an antibody (e.g., scFv), the antibody (e.g., scFv) includes one or both of a VH FR1 and VH FR2, wherein the VH FR1 comprises the amino acid sequence of QVQLVX¹SGGGLVQPGGSLRLSCAASGFTFS (SEQ ID NO: 1), wherein X¹ is an amino acid other than E, and in certain embodiments X¹ is Q; the VH FR2 is a comprises the amino acid sequence WVRQAPGKGLEWV (SEQ ID NO: 202). Such antibodies (e.g., scFv) may also include one or more of a VL FR1 comprising the amino acid sequence of SEQ ID NO:3, a VL FR2 comprising the amino acid sequence of SEQ ID NO: 4, and a VL FR3 comprising an amino acid sequence of SEQ ID NO:5, and a VL FR3 comprising the amino acid sequence of SEQ ID NO:6. In one embodiment such antibodies (e.g., scFv) comprise one or more of any combination of a VH FR1, VH FR2, VH FR3, VL FR1, VL FR2, and VL FR3 of Table 1.

In further embodiments, the present disclosure provides polypeptides, which polypeptides may be present in an antibody (e.g., an scFv), that bind Protein A, and comprise an amino acid sequence selected from SEQ ID NOS: 177-197 as set out FIG. 5.

In one embodiment, the polypeptide, which polypeptide may be present in an antibody (e.g., an scFv), comprises the amino acid sequence of one of SEQ ID NOS: 177-197.

In one embodiment, such polypeptides comprise, from N- to C-terminus,

a VH CDR1 comprising an amino acid sequence of SYAMH (SEQ ID NO:17);

a Protein A binding sequence of one of SEQ ID NOS: 177-197; and

a VH CDR3 comprising an amino acid sequence of ASVGATGPFDI (SEQ ID NO: 19).

Where a polypeptide of SEQ ID NOS: 177-197 is present in an antibody (e.g., an scFv), the antibody may comprise a VL CDR1 comprising the amino acid sequence QGDSLRSYYAS (SEQ ID NO: 20), a VL CDR2 comprising the amino acid sequence GENNRPS (SEQ ID NO: 21) and a VL CDR3 comprising the amino acid sequence NSRDSSGTHLTV (SEQ ID NO: 22). In one embodiment where the polypeptide is an antibody (e.g., scFv), the antibody (e.g., scFv) includes one or both of a VH FR1 and VH FR2, wherein the VH FR1 comprises the amino acid sequence of QVQLVX¹SGGGLVQPGGSLRLSCAASGFTFS (SEQ ID NO: 1), wherein X¹ is an amino acid other than E, and in certain embodiments X¹ is Q; the VH FR2 is a comprises the amino acid sequence WVRQAPGKGLEWV (SEQ ID NO: 202). Such antibodies (e.g., scFv) may also include one or more of a VL FR1 comprising the amino acid sequence of SEQ ID NO:3, a VL FR2 comprising the amino acid sequence of SEQ ID NO: 4, and a VL FR3 comprising an amino acid sequence of SEQ ID NO:5, and a VL FR3 comprising the amino acid sequence of SEQ ID NO:6. In one embodiment such antibodies (e.g., scFv) comprise one or more of any combination of a VH FR1, VH FR2, VH FR3, VL FR1, VL FR2, and VL FR3 of Table 1.

In certain embodiments, an Ab disclosed herein is an internalizing anti-EphA2 Ab. Binding of such an Ab to the ECD of an EphA2 molecule present on the exterior surface of a living cell under appropriate conditions results in internalization of the Ab, which is characterized by the transport of the Ab into the cell-membrane-bound interior of the cell. Internalizing Abs find use, e.g., as vehicles for targeted delivery of drugs, toxins, enzymes, nanoparticles (e.g., liposomes), DNA, etc., e.g., for therapeutic applications.

Certain Abs described herein are single chain Fv Abs e.g., scFvs or (scFv′)₂s. In such Abs, VH and VL polypeptides are joined to each other in either of two orientations (i.e., with the VH N-terminal to the VL, or with the VL N-terminal to the VH) either directly or via an amino acid linker. Such a linker may be, e.g., from 1 to 50, 5 to 40, 10 to 30, or 15 to 25 amino acids in length. In certain embodiments, 80% or greater, 85% or greater, 90% or greater, 95% or greater, or 100% of the residues of the amino acid linker are serine (S) and/or glycine (G). Examples of suitable scFv linkers may be or include the sequence:

(SEQ ID NO: 41)
ASTGGGGSGGGGSGGGGSGGGGS,
(SEQ ID NO: 42)
GGGGSGGGGSGGGGSGGGGS,
(SEQ ID NO: 43)
GGGGSGGGGSGGGGS,
(SEQ ID NO: 44)
ASTGGGGAGGGGAGGGGAGGGGA,
(SEQ ID NO: 45)
GGGGAGGGGAGGGGAGGGGA,
(SEQ ID NO: 46)
TPSHNSHQVPSAGGPTANSGTSGS,
or
(SEQ ID NO: 47)
GGSSRSSSSGGGGSGGGG.

Examples of an internalizing anti-EphA2 Ab include scFv TS1:

(SEQ ID NO: 48)
QVQLVQSGGGLVQPGGSLRLSCAASGFTFSSYAMHWVRQAPGKGLEWVAV
ISYDGSNKYYADSVKGRFTISRDNSKNTLYLQMNSLRAEDTAVYYCARAS
VGATGPFDIWGQGTLVTVSSASTGGGGSGGGGSGGGGSGGGGSSSELTQP
PSVSVAPGQTVTITCQGDSLRSYYASWYQQKPGTAPKLLIYGENNRPSGV
PDRFSGSSSGTSASLTITGAQAEDEADYYCNSRDSSGTHLTVFGGGTKLT
VLG.

In TS1 above, the CDRs are underlined and are presented in the following order: VH CDR1, VH CDR2, VH CDR3, VL CDR1, VL CDR2, and VL CDR3. Thus, in scFv TS1, and in certain other scFvs disclosed herein, the VH of the scFv is at the amino terminus of the scFv and is linked to the VL by a linker (here in italics).

Also provided are variants of scFv TS1 in which all framework regions and CDRs of TS1 are unchanged except for VH CDR2, which is replaced with a CDR selected from any of the 18 different CDRH2 sequences set forth above in Table 2B.

Methods of Ab Production

Using the information provided herein, the Abs disclosed herein may be prepared using standard techniques. For example, the amino acid sequences provided herein can be used to determine appropriate nucleic acid sequences encoding the Abs and the nucleic acids sequences then used to express one or more of the Abs. The nucleic acid sequence(s) can be optimized to reflect particular codon “preferences” for various expression systems according to standard methods.

Using the sequence information provided herein, the nucleic acids may be synthesized according to a number of standard methods. Oligonucleotide synthesis, is conveniently carried out on commercially available solid phase oligonucleotide synthesis machines or manually synthesized using, for example, the solid phase phosphoramidite triester method. Once a nucleic acid encoding an Ab disclosed herein is synthesized, it can be amplified and/or cloned according to standard methods.

Expression of natural or synthetic nucleic acids encoding the Abs disclosed herein can be achieved by operably linking a nucleic acid encoding the Ab to a promoter (which may be constitutive or inducible), and incorporating the construct into an expression vector to generate a recombinant expression vector. The vectors can be suitable for replication and integration in prokaryotes, eukaryotes, or both. Typical cloning vectors contain functionally appropriately oriented transcription and translation terminators, initiation sequences, and promoters useful for regulation of the expression of the nucleic acid encoding the Ab. The vectors optionally contain generic expression cassettes containing at least one independent terminator sequence, sequences permitting replication of the cassette in both eukaryotes and prokaryotes, e.g., as found in shuttle vectors, and selection markers for both prokaryotic and eukaryotic systems.

To obtain high levels of expression of a cloned nucleic acid it is common to construct expression plasmids which contain a strong promoter to direct transcription, a ribosome binding site for translational initiation, and a transcription/translation terminator, each in functional orientation to each other and to the protein-encoding sequence. The Ab gene(s) may also be subcloned into an expression vector that allows for the addition of a tag sequence, e.g., FLAG™ or His6, at the C-terminal end or the N-terminal end of the Ab (e.g. scFv) to facilitate identification, purification and manipulation. Once the nucleic acid encoding the Ab is isolated and cloned, one can express the nucleic acid in a variety of recombinantly engineered cells. Examples of such cells include bacteria, yeast, filamentous fungi, insect, and mammalian cells.

Isolation and purification of an Ab disclosed herein can be accomplished by isolation from culture supernatant from cells secreting the Ab followed by purification using affinity chromatography, or can be isolated from a lysate of cells genetically modified to express the protein constitutively and/or upon induction, or from a synthetic reaction mixture, with purification, e.g., by affinity chromatography (e.g., using Protein A or Protein G). The isolated Ab can be further purified by dialysis and other methods normally employed in protein purification.

The present disclosure also provides cells that produce subject Abs. For example, the present disclosure provides a recombinant host cell that is genetically modified with one or more nucleic acids comprising nucleotide sequence encoding an Ab disclosed herein, or a polypeptide chain thereof. DNA is cloned into, e.g., a bacterial (e.g., bacteriophage), yeast (e.g. Saccharomyces or Pichia) insect (e.g., baculovirus) or mammalian expression system. One suitable technique uses a filamentous bacteriophage vector system. See, e.g., U.S. Pat. No. 5,885,793; U.S. Pat. No. 5,969,108; and U.S. Pat. No. 6,512,097.

Ab Modification

The present disclosure encompasses Abs (and nucleic acids encoding Abs) that are modified to provide a desired feature, e.g., to facilitate delivery to a specific type of tissue and/or cells in a subject (e.g., to a tumor), to increase serum half-life, to supplement anti-cancer activity, etc. The Abs disclosed herein can be provided with or without modification, and are exemplified as human Abs, but can be readily modified and prepared as humanized Abs or chimeric Abs. One way to modify an Ab (e.g., an scFv) is to conjugate (e.g. link) one or more additional elements at an N- and/or C-terminus of a polypeptide chain comprised by the Ab, or to any amino acid (including an internal amino acid) of the Ab. Such additional elements include, e.g., a liposome, another protein, a detectable label (e.g., e.g., a radioisotope an enzyme which generates a detectable product, a fluorescent protein, a chromogenic protein, dye, a fluorescence emitting metal, a chemiluminescent compound and the like), a drug, and/or carrier molecule. Optionally such conjugation is via a linker moiety connecting the Ab to the additional element(s).

An Ab disclosed herein modified with one or more additional elements retains its binding specificity, while exploiting properties of the one or more additional elements to impart one or more additional characteristics.

In certain embodiments, an Ab disclosed herein is formulated by attachment to a therapeutic moiety, such as to the exterior of a lipidic nanoparticle (e.g., a liposome, a solid lipidic nanoparticle, or a micelle) or to a chemotherapeutic agent such as a maytansinoid (e.g., DM1, the chemotherapeutic moiety of ado-trastuzumab emtansine) or an auristatin (e.g., monomethyl auristatin E, the chemotherapeutic moiety of brentuximab vedotin. When attached to a lipidic microparticle, the Ab may be covalently attached to a polymer (e.g., to a terminus of the polymer) that is conjugated to a lipid suitable for insertion into a lipidic membrane. Ab-conjugated liposomes are referred to herein as “immunoliposome(s)”. The Abs disclosed herein can act as an immunoliposome targeting component, enabling the immunoliposome to specifically bind to EphA2 on the surface of a cancer cell. The immunoliposomes can be loaded to contain one or more of anti-cancer agents, such as a small molecule (e.g., a chemotherapeutic agent), or a nucleic acid (e.g. an siRNA). Methods of making and loading lipidic nanoparticles, such as liposomes and immunoliposomes, are described in, e.g., US 2010/0068255, US 2010/0008978, US 2009/0171077, US 2009/0155272, US 2007/0116753, US 2007/0110798, US 2007/0031484, US 2006/0147513, US 2005/0112065, US 2004/0037874, US 2004/0209366, US 2003/0003143, U.S. Pat. No. 7,135,177, U.S. Pat. No. 7,022,336, U.S. Pat. No. 6,803,053, U.S. Pat. No. 6,528,087, U.S. Pat. No. 6,214,388, U.S. Pat. No. 6,210,707, U.S. Pat. No. 6,110,491, U.S. Pat. No. 5,980,935, U.S. Pat. No. 5,380,531, U.S. Pat. No. 7,507,407, U.S. Pat. No. 7,479,276, and U.S. Pat. No. 7,462,603.

Any of the above elements that are attached to the Ab may be linked to the Ab via a linker, e.g. a flexible linker, e.g. a peptide linker or a polyethylene glycol linker. If present, the linker molecules are generally of sufficient length to permit the Ab and a linked moiety to allow some flexible movement of the Ab when bound, e.g., to the surface of a lipidic nanoparticle.

### Compositions

Also provided by the present disclosure are compositions, e.g., compositions that include any of the Abs or conjugates described herein, or nucleic acids encoding any of the Abs disclosed herein. In certain aspects, the compositions find use in treating cancer in a subject (e.g., a human), and may be suitable for treatment during any stage of the disease. Compositions containing one, two, or more different Abs can be provided as a pharmaceutical composition and administered to a mammal (e.g., to a human) in need thereof.

As described above, the liposome may contain one or more Abs that are different than the Abs disclosed herein. The liposome may be dual-specific, polyspecific, etc., in that the liposome has a plurality of different Abs attached and is specific for at least one additional epitope or antigen in addition to the EphA2 epitope bound by an Ab disclosed herein.

Combinations can be provided in a single formulation or can be provided as separate formulations, e.g., in separate containers which may in turn be comprised in a larger container as a kit, where the separate formulations may contain, e.g., a single Ab or a plurality of different Abs. Such separate formulations may be combined prior to administration or administered by separate injection.

An Ab or immunoliposome disclosed herein can be formulated in a pharmaceutically acceptable carrier, e.g., formulated for parenteral administration for use in the methods described. In certain embodiments, e.g., where an Ab is administered as a liquid injectable (e.g., suitable for intravenous injection) an Ab formulation may be a sterile, non-pyrogenic aqueous solution comprising one, or more, or all of salts (e.g., to adjust tonicity) buffers, preservatives, amino acids, and other pharmaceutically acceptable carriers and excipients, and may be provided, e.g., as a ready-to-use dosage form, or as a reconstitutable storage-stable powder or liquid. Formulation for administration via convection enhanced delivery may be as described in, e.g., US 20090208422.

Compositions of the present disclosure can include a therapeutically effective amount of a subject antibody, as well as any other compatible components, as needed. By “therapeutically effective amount” is meant that the administration of that amount to an individual, either in a single dose, as part of a series of the same or different antibody or compositions, is effective to reduce the proliferation and/or metastases of a cancerous cell in a subject or to provide any other detectable therapeutic benefit. Such therapeutically effective amount of an antibody and its impact on cell growth includes cooperative and/or synergistic inhibition of cell growth in conjunction with one or more other therapies (e.g., immunotherapy, chemotherapy, radiation therapy etc.). As noted below, the therapeutically effective amount can be adjusted in connection with dosing regimen and diagnostic analysis of the subject's condition (e.g., monitoring for the presence or absence of a cell surface epitopes using an antibody specific for EphA2) and the like.

Amount and Dosage

The exact dose will be ascertainable by one skilled in the art. The dosage can depend on a variety of factors including the strength of the particular compound employed, the condition of the subject, and the body weight of the subject, as well as the severity of the illness and the stage of the disease. The dosage will also be determined by the existence, nature, and extent of any adverse side-effects that might accompany the administration of a particular compound. As known in the art, adjustments based on age, body weight, sex, diet, time of administration, drug interaction and severity of condition may be necessary and will be ascertainable with routine experimentation by those skilled in the art. A therapeutically effective amount is also one in which the therapeutically beneficial effects outweigh any toxic or detrimental effects of the antibody.

The amount of composition administered to a subject, e.g., a human, in the context of the present disclosure should be sufficient to effect a prophylactic or therapeutic response in the animal over a reasonable time frame, and varies depending upon the goal of the administration, the health and physical condition of the individual to be treated, age, the degree of resolution desired, the formulation of the antibody composition, the treating clinician's assessment of the medical situation, and other relevant factors. Thus it is expected that the amount will fall in a relatively broad range, but can nevertheless be routinely determined through various features of the subject such as note above.

As an example, a non-limiting range for a therapeutically or prophylactically effective amount of a subject antibody is from about 0.1 mg/kg to about 20 mg/kg, e.g., from about 1 mg/kg to about 10 mg/kg.

The concentration of an antibody in a pharmaceutical formulation can vary from less than about 0.1%, usually at or at least about 2% to as much as 20% to 50% or more by weight, and will be selected primarily by consideration of fluid volumes, viscosities, etc., in accordance with the particular mode of administration selected and the patient's needs.

Also, suitable doses and dosage regimens can be determined by comparisons to anticancer or immunosuppressive agents that are known to affect the desired growth inhibitory or immunosuppressive response. Such dosages include dosages which result in the low dose inhibition of cell growth, without significant side effects. In proper doses and with suitable administration of certain compounds, the compounds of the present disclosure can provide for a wide range of intracellular effects, e.g., from partial inhibition to essentially complete inhibition of cell growth. Dosage treatment may be a single dose schedule or a multiple dose schedule (e.g., including ramp and maintenance doses). As indicated below, a subject composition may be administered in conjunction with other agents, and thus doses and regiments can vary in this context as well to suit the needs of the subject

Combination Therapy

Any of a wide variety of cancer therapies can be combined in a composition with an antibody of the present disclosure. For example, agents used in chemotherapeutic treatment or biological response modifier treatment may be present in the pharmaceutical composition comprising the antibody, such as an immunoliposome.

Chemotherapeutic agents are non-proteinaceous compounds that reduce proliferation of cancer cells, and encompass cytotoxic agents and cytostatic agents. Non-limiting examples of chemotherapeutic agents include alkylating agents, nitrosoureas, antimetabolites, antitumor antibiotics, plant (e.g., vinca) alkaloids, nucleic acids, such as inhibitory nucleic acids (e.g. siRNA), and steroid hormones.

Antimetabolite agents include folic acid analogs, pyrimidine analogs, purine analogs, and adenosine deaminase inhibitors, for example.

Suitable natural products and their derivatives, (e.g., vinca alkaloids, antitumor antibiotics, enzymes, lymphokines, and epipodophyllotoxins) can be used as anti-cancer agents. For example, taxanes, such as paclitaxel, as well as any active taxane derivative such as docetaxel, or a taxane pro-drug such as is 2′-(2-(N,N′-diethylamino)propionyl)-paclitaxel, 7-(2-(N, N′-diethylamino)propionyl)-paclitaxel, 2′-(2-(N, N′-diethylamino)propionyl)-docetaxel or 7-(2-(N, N′-diethylamino)propionyl)-docetaxel, are suitable.

Other anti-proliferative cytotoxic agents are navelbene, CPT-11 (irinotecan), anastrazole, letrazole, capecitabine, reloxafine, cyclophosphamide, ifosamide, and droloxafine. Microtubule affecting agents that have antiproliferative activity are also suitable for use. Hormone modulators and steroids (including synthetic analogs) are suitable for use.

### Methods

Therapeutic Methods

An antibody of the present disclosure finds therapeutic use in a variety of cancers. Subjects having, suspected of having, or at risk of developing cancer are contemplated for therapy and diagnosis described herein.

By “treatment” is meant that at least an amelioration of the symptoms associated with the condition afflicting the host is achieved, where amelioration is used in a broad sense to refer to at least a reduction in the magnitude of a parameter, e.g. symptom, associated with the condition being treated. As such, treatment also includes situations where the pathological condition, or at least symptoms associated therewith, are completely inhibited, e.g., prevented from happening, or stopped, e.g. terminated, such that the host no longer suffers from the condition, or at least the symptoms that characterize the condition. Thus treatment includes: (i) prevention, that is, reducing the risk of development of clinical symptoms, including causing the clinical symptoms not to develop, e.g., preventing disease progression to a harmful state; (ii) inhibition, that is, arresting the development or further development of clinical symptoms, e.g., mitigating or completely inhibiting an active disease, e.g., so as to decrease tumor load, which decrease can include elimination of detectable cancerous cells (e.g. metastatic cancer cells); and/or (iii) relief, that is, causing the regression of clinical symptoms.

A variety of subjects are treatable according to the methods. Generally such subjects are “mammals” or “mammalian,” where these terms are used broadly to describe organisms which are within the class mammalia, including the orders carnivore (e.g., dogs and cats), rodentia (e.g., mice, guinea pigs, and rats), and primates (e.g., humans, chimpanzees, and monkeys). In many embodiments, the subjects will be humans.

In a related embodiment, the subject being treated has a cancer expresses (e.g. overexpresses) a tumor associated antigen, and/or EphA2. The antigen is expressed on the cancer cell surface and is often present at a higher level than a corresponding non-cancerous cell. This aspect can be beneficial in the context of the methods of the present disclosure in that cells expressing or presenting EphA2 can be amenable to treatment with an antibody of the present disclosure. The antibody can be administered to a subject, for example, where therapy is initiated at a point where presence of the antigen is not detectable, and thus is not intended to be limiting. It is also possible to initiate antibody therapy prior to the first sign of disease symptoms, at the first sign of possible disease, or prior to or after diagnosis of a disease

Cancer

An anti-EphA2 antibody composition and/or anti-EphA2 antibody conjugate composition of the present disclosure may be used in an anti-cancer therapy, particularly where the cancerous cells present EphA2 on an extracellularly accessible cell surface. For example, antibody compositions and/or therapeutic antibody conjugate compositions described herein can be administered to a subject (e.g. a human patient) to reduce proliferation of cancerous cells, e.g., to reduce tumor size, reduce cancer load, reduce metastasis, and/or improve the clinical outcome in patients. The methods relating to cancer contemplated herein include, for example, use of antibody therapy alone or in combination with anti-cancer vaccine or therapy.

Cancers particularly amenable to antibody therapy can be identified by methods similar to the diagnostic methods described herein and others known in the art.

Where the anti-cancer therapy includes administration of an antibody composition described previously, the anti-cancer therapy can be particularly directed to cancerous cells expressing cell-surface accessible and/or solvent-exposed epitopes bound by the subject antibodies, including metastatic cancer.

Examples of EphA2-expressing cancers amenable to therapy include, solid tumors, semisolid tumors, and liquid tumors. Examples of EphA2-expressing cancers amenable to therapy include, but are not necessarily limited to, EphA2 positive cancers of breast, brain, ovary, urinary bladder, prostate, pancreas, esophagus, lung (e.g., non-small cell lung cancer), and stomach, and can include colon, vulva, skin (e.g., melanoma), kidney, and gliomas (e.g., glioblastoma multiforme, astrocytoma), leukemia, and lymphoma.

It should be noted that while EphA2 may be expressed at higher levels on a cancer cell compared to a non-cancerous cell, this is not a limitation of the therapies disclosed herein.

EphA2-expressing carcinomas that can be amenable to therapy by a method disclosed herein include, but are not limited to, esophageal carcinoma, hepatocellular carcinoma, basal cell carcinoma (a form of skin cancer), squamous cell carcinoma (various tissues), bladder carcinoma, including transitional cell carcinoma (a malignant neoplasm of the bladder), bronchogenic carcinoma, colon carcinoma, colorectal carcinoma, gastric carcinoma, lung carcinoma, including small cell carcinoma and non-small cell carcinoma of the lung, adrenocortical carcinoma, thyroid carcinoma, pancreatic carcinoma, breast carcinoma, ovarian carcinoma, prostate carcinoma, adenocarcinoma, sweat gland carcinoma, sebaceous gland carcinoma, papillary carcinoma, papillary adenocarcinoma, cystadenocarcinoma, medullary carcinoma, renal cell carcinoma, ductal carcinoma in situ or bile duct carcinoma, choriocarcinoma, seminoma, embryonal carcinoma, Wilm's tumor, cervical carcinoma, uterine carcinoma, testicular carcinoma, osteogenic carcinoma, epithelieal carcinoma, and nasopharyngeal carcinoma.

EphA2-expressing sarcomas that can be amenable to therapy by a method disclosed herein include, but are not limited to, fibrosarcoma, myxosarcoma, liposarcoma, chondrosarcoma, chordoma, osteogenic sarcoma, osteosarcoma, angiosarcoma, endotheliosarcoma, lymphangiosarcoma, lymphangioendotheliosarcoma, synovioma, mesothelioma, Ewing's sarcoma, leiomyosarcoma, rhabdomyosarcoma, and other soft tissue sarcomas.

Additional EphA2-expressing solid tumors that can be amenable to therapy by a method disclosed herein include, but are not limited to, glioma, astrocytoma, medulloblastoma, craniopharyngioma, ependymoma, pinealoma, hemangioblastoma, acoustic neuroma, oligodendroglioma, menangioma, melanoma, neuroblastoma, and retinoblastoma.

EphA2-expressing leukemias that can be amenable to therapy by a method disclosed herein include, but are not limited to, a) chronic myeloproliferative syndromes (neoplastic disorders of multipotential hematopoietic stem cells); b) acute myelogenous leukemias (neoplastic transformation of a multipotential hematopoietic stem cell or a hematopoietic cell of restricted lineage potential; c) chronic lymphocytic leukemias (CLL; clonal proliferation of immunologically immature and functionally incompetent small lymphocytes), including B-cell CLL, T-cell CLL prolymphocytic leukemia, and hairy cell leukemia; and d) acute lymphoblastic leukemias (characterized by accumulation of lymphoblasts). Lymphomas that can be treated using a method include, but are not limited to, B-cell lymphomas (e.g., Burkitt's lymphoma); Hodgkin's lymphoma; non-Hodgkin's lymphoma, and the like.

Other cancers that can be amenable to treatment according to the methods disclosed herein include atypical meningioma (brain), islet cell carcinoma (pancreas), medullary carcinoma (thyroid), mesenchymoma (intestine), hepatocellular carcinoma (liver), hepatoblastoma (liver), clear cell carcinoma (kidney), and neurofibroma mediastinum.

Other examples of cancers that can be amenable to treatment using a methods disclosed herein include, but are not limited to, EphA2-expressing cancers of epithelial and neuroectodermal origin. Examples of EphA2-expressing cancers of epithelial origin include, but are not limited to, small cell lung cancer, cancers of the breast, eye lens, colon, pancreas, kidney, liver, ovary, and bronchial epithelium. The methods of the present disclosure may be used to treat cancer cells known to overexpress EphA2.

Examples of EphA2-expressing cancers of neuroectodermal origin include, but are not limited to, Ewings sarcoma, spinal tumors, brain tumors, supratenbrial primative neuroectodermal tumors of infancy, tubulocystic carcinoma, mucinous tubular and spindle cell carcinoma, renal tumors, mediastinum tumors, neurogliomas, neuroblastomas, and sarcomas in adolescents and young adults.

Combinations with Other Cancer Therapies

As noted above, another feature of the methods is that an antibody can be administered to the subject in combination with one or more other therapies. Such therapy may be combined in a composition or be conjugated to the subject antibodies. In addition to being physically combined with antibodies disclosed herein (e.g, as a conjugate or in a liposome or other lipidic nanoparticle), one or more anti-cancer agents, such as those listed in Table 4 above, may be administered in conjunction with, either simultaneously or before or after, administration of an antibody disclosed herein.

A therapy or treatment other than administration of antibody composition can be administered anywhere from simultaneously, to up to 5 hours or more, e.g., 10 hours, 15 hours, 20 hours or more, prior to or after administration of a subject antibody. A subject antibody and other therapeutic intervention are administered or applied sequentially, e.g., where a subject antibody is administered before or after another therapeutic treatment. A subject antibody and other therapy are administered simultaneously, e.g., where a subject antibody and a second therapy are administered at the same time, e.g., when the second therapy is a drug it can be administered along with a subject antibody as two separate formulations or combined into a single composition that is administered to the subject. Regardless of whether administered sequentially or simultaneously, as illustrated above, the treatments are considered to be administered together or in combination for purposes of the present disclosure.

Additional standard anti-cancer therapeutics that may or may not be administered in conjunction with a subject antibody, include but not limited to immunotherapy, chemotherapeutic agents and surgery (e.g., as those described further below). In addition, therapeutic administration of a subject antibody can also be post-therapeutic treatment of the subject with an anti-cancer therapy, where the anti-cancer therapy can be, for example, surgery, radiation therapy, administration of chemotherapeutic agents, and the like. Antibodies other than those disclosed herein, particularly monoclonal antibodies that can provide for complement-mediated killing, and/or antibody-dependent cellular cytotoxicity-mediated killing, of a target cell may also be used.

For example, a subject antibody can be administered in combination with one or more chemotherapeutic agents (e.g., cyclophosphamide, doxorubicin, vincristine and prednisone (CHOP)), and/or in combination with radiation treatment and/or in combination with surgical intervention (e.g., pre- or post-surgery to remove a tumor), radiation therapy, bone marrow transplantation, biological response modifier treatment, and certain combinations of the foregoing. Radiation therapy includes, but is not limited to, X-rays or gamma rays that are delivered from either an externally applied source such as a beam, or by implantation of small radioactive sources.

Routes of Administration

In practicing the methods, routes of administration (path by which a subject antibody is brought into a subject) may vary, where representative routes of administration for a subject antibody are described in greater detail below. A subject antibody alone or in combinations described above can be administered systemically (e.g., by parenteral, intravenous, intramuscular, intrathecal, intraventricular, or subcutaneous administration) or locally (e.g., at a local tumor site, e.g., by intratumoral administration (e.g., into a solid tumor, into an involved lymph node in a lymphoma or leukemia, or by convection enhanced delivery, e.g. into the brain, e.g., as disclosed in US 20090209937), administration into a blood vessel supplying a solid tumor, etc.), into a body cavity or lumen, or into an organ. These different routes of administration may be carried out by injection or infusion.

Formulations suitable for parenteral administration include aqueous and non-aqueous, isotonic sterile injection solutions, which can contain anti-oxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The formulations can be presented in unit-dose or multi-dose sealed containers, such as ampules and vials, and can be stored in a freeze-dried (lyophilized) condition requiring only the addition of the sterile liquid excipient, for example, water, for injections, immediately prior to use. Injection solutions and suspensions can be prepared from sterile powders, granules, and tablets of the kind previously described. Methods for preparing parenterally administrable compositions will be known or apparent to those skilled in the art.

Administration of the therapy can be repeated over a desired period, e.g., repeated over a period of about 1 day to about 5 days or once every several days, for example, about five days, over about 1 month, about 2 months, etc. It also can be administered prior, at the time of, or after other therapeutic interventions, such as surgical intervention to remove cancerous cells. The antibody can also be administered as part of a combination therapy, in which at least one of an immunotherapy, a cancer chemotherapy or a radiation therapy is administered to the subject (as described in greater detail above).

Detection Methods

The present disclosure provides a method of detecting EphA2, e.g., on an EphA2-expressing cell in a biological sample in a subject or in a sample isolated from a subject. The methods are useful to both diagnostic and prognostic purposes. A subject method generally involves contacting a sample containing a cell with an antibody or antibody conjugate of the present disclosure; and detecting binding of the antibody or antibody conjugate to a cell in the sample. The cell can be in vitro, where the cell is in a biological sample obtained from a subject suspected for having cancer cells, a subject undergoing cancer treatment, or a subject being tested for susceptibility to treatment. The cell can be in vivo, e.g., the cell is in a subject suspected for having cancer cells, a subject undergoing treatment, or a subject being tested for susceptibility to treatment.

The detection assays described herein can be used to determine whether a subject has a cancer that is more or less amenable to therapy using antibody-based therapy, as well as monitor the progress of treatment in a subject. It also may be used to assess the course of other combination therapies. Thus, the diagnostic assays can inform selection of therapy and treatment regimen by a clinician.

Antibodies can be used to detect cells expressing EphA2 in a biological sample of a subject having or suspected of having EphA2-expressing cancerous cells via immunodiagnostic techniques. Such diagnostics can be useful to identify patients amenable to the therapies disclosed later below, and/or to monitor response to therapy.

Suitable immunodiagnostic techniques include, but are not necessarily limited to, both in vitro and in vivo (imaging) methods. For example, anti-EphA2 antibodies can be detectably labeled, administered to a subject suspected of having a cancer characterized by cell surface expression of EphA2, and bound detectably labeled antibody detected using imaging methods available in the art.

The phrase “in vivo imaging” as used herein refers to methods of detecting the presence of an antibody (e.g. detectably labeled 2D6) in whole, live mammal. Optically detectable proteins such as fluorescent antibodies and luciferases-conjugated antibodies may be detected by in vivo imaging. In vivo imaging may be used to provide 2-D as well as 3-D images of a mammal. Radiolabeled antibodies, for example, may be administered to a subject and the subject imaged with a gamma camera. Charge-coupled device cameras, CMOS, or 3D tomographers may used to carry out in vivo imaging. Methods of in vivo imaging using computed tomography, magnetic resonance imaging, ultrasonography, positron emission tomography, single-photon emission computed tomography (SPECT), and the like are well known in the art. The information from many in vivo imaging methods as those described above can provide information on cancer cells in the subject.

Where the methods are in vitro, the biological sample can be any sample in which a cancer cell may be present, including but not limited to, blood samples (including whole blood, serum, etc.), tissues, whole cells (e.g., intact cells), and tissue or cell extracts. For example, the assay can involve detection of EphA2 on live cells or cells in a histological tissue sample. Particularly, detection can be assessed on an extracellular surface of a living cell. For example, the tissue sample may be fixed (e.g., by formalin treatment) and may be provided embedded in a support (e.g., in paraffin) or frozen unfixed tissue.

Assays can take a wide variety of forms, such as competition, direct reaction, or sandwich type assays. Examples include Western blots; agglutination tests; enzyme-labeled and mediated immunoassays, such as enzyme-linked immunosorbant assays (ELISAs); biotin/avidin type assays; radioimmunoassays; immunoelectrophoresis; immunoprecipitation, and the like. The reactions generally include detectable labels conjugated to the antibody. Labels include those that are fluorescent, chemiluminescent, radioactive, enzymatic and/or dye molecules, or other methods for detecting the formation of a complex between antigen in the sample and the antibody or antibodies reacted therewith. Where a solid support is used, the solid support is usually first reacted with a solid phase component under suitable binding conditions such that the antibody is sufficiently immobilized to the support. Coupling agents, such as proteins (e.g., serum albumins (e.g, bovine serum albumin (BSA)), keyhole limpet hemocyanin, immunoglobulin molecules, thyroglobulin, ovalbumin) polysaccharides, polylactic acids, polyglycolic acids, polymeric amino acids, amino acid copolymers and the like can be used to facilitate immobilization to the solid support.

Assays can be conducted in solution, such that the antibodies and the antigens form complexes under precipitating conditions. An antibody-coated particle can be contacted under suitable binding conditions with a biological sample suspected of containing the target antigen to provide for formation of particle-antibody-antigen complex aggregates which can be precipitated and separated from the sample using washing and/or centrifugation. The reaction mixture can be analyzed to determine the presence or absence of antibody-antigen complexes using any of a number of standard methods, such as those immunodiagnostic methods described above.

The assays of the present disclosure can include assays detecting cellular uptake of an antibody and/or antibody conjugate of the present disclosure by live cells, e.g., as a method of detecting EphA2-expressing cancerous cells. In certain embodiments, where the antibodies and/or antibody conjugates of the present disclosure are internalized by cells expressing EphA2, a sample suspected of containing cells expressing EphA2 can be contacted with such an antibody and incubated for a time sufficient to allow for internalization of the antibody and/or antibody conjugate. Following washing. The internalized antibodies may be detected via its label as contained with the cells (e.g. FACS, spectrometer, radioisotope counter, etc.). Internalizing antibodies may also be selected for as described in U.S. Pat. No. 7,045,283.

The above-described assay reagents, including the antibodies of the present disclosure, can be provided in kits, with suitable instructions and other necessary reagents, in order to conduct immunoassays as described above. The kit can also contain, depending on the particular immunoassay used, suitable labels and other packaged reagents and materials (i.e. wash buffers and the like). Standard immunoassays, such as those described above, can be conducted using these kits.

The following examples are offered as illustration but not limitation.

### Examples

### Example 1—D2-1A7 Thermostability and Protein a Binding

D2-1A7 scFv was profiled for thermostability and for binding to a Protein A resin.

Thermostability determination was by Differential scanning fluorescence (DSF), performed as follows: 25 μl of 10 μM scFv and 1× Sypro® Orange (Sigma) in 1× PBS was heated from 20° C. to 90° C. at a rate of 1° C./min. and resulting fluorescence data were collected. Heating and fluorescence data generation was carried out using the 105 real time detection system (Bio-Rad®). Data were analyzed using GraphPad® Prism software. The Tₘ reported is the temperature at the maximum value of the first derivative.

Protein A binding for multiple samples was determined as follows: scFvs expressed by mammalian cells were obtained from cultures by centrifugation at 4000 g for 15 minutes to remove cells. The scFv titer in the supernatant was determined using Fortebio. Protein A binding was assayed using a 96 well Protein A plate (Protein A 96 well protein purification plate (GE Healthcare: Life Sciences, Protein A HP MultiTrap®) which contains Protein A-conjugated resin in wells with porous bottoms, allowing liquid in the wells to be collected from underneath via centrifugation or suction. Each well of the plate was washed with 1×PBS, and supernatant containing 250 μg of protein was loaded into each well. The supernatant is then separated from the Protein A resin using a vacuum manifold (Whatman®) with 10 mbar of pressure and the flow-through from each well separately collected into empty wells of a standard 96 well plate. The wells of the Protein A plate were then washed with 600 μL of 1×PBS (pH 7.6). To elute the resin-bound Ab, 200 μL of 0.1M acetic acid was added to each well and incubated at room temperature for 3-10 minutes. The plate was placed over a second plate to capture eluate and centrifuged at 100×g for 2 minutes, and 20 μL of 1M Tris, pH 8.0 was added to neutralize the Abs in the eluate. To determine the percentage of Protein A binding, the concentration of scFv remaining in the flow-through was compared to the amount of protein (250 ug) loaded originally.

CONCLUSIONS: The measured Tₘ of 65° C. is unacceptably low for efficient and commercially productive liposome conjugation and the measured Protein A binding of less than 10% is unacceptably low for commercial production.

### Example 2—Framework Engineering to Improve Tₘ

As an initial approach to increasing stability characteristics, including Tₘ, the CDRs of D2-1A7 were grafted to different Ig frameworks. The framework of the heavy chain variable region of D2-1A7 is IGHV3-30. VH3s are generally very stable and typically have the ability to bind to Protein A, and are commonly used frameworks for therapeutic Abs. It was therefore decided to retain the heavy chain within the VH3 family, but to graft the heavy chain of D2-1A7 onto an IGHV3-23 backbone, which is the most common isotype. Additionally, two stabilizing mutations were made within the IGHV3-23 framework: E6Q and S49A.

The framework of the light chain variable region of D2-1A7 is IGVA3-19, which is not commonly found in the natural human Ab repertoire, so it was decided to graft the D2-1A7 light chain CDRs to the more common framework IGVA1-40, which is also found frequently paired with IGVH3-23. Two thermostable variants, TS1 and TS2, were designed. Based on homology modeling, positions 1 and 22 were thought to be interacting with the antigen, and so both variants were back-mutated to have the residues occurring at these positions in D2-1A7 (Q1S, S22T). In TS1, a hydrophobic pocket was retained which was believed to be important for stability (G13V, L78A), while adjusting charge on the surface (V3E, R18T). The amino acid sequence was compared to the repertoire of naïve sequences and consensus amino acids including L39K (TS1) F65S (both), K66G (TS2) or K66S (TS1), and A74T (TS1) were introduced.

TABLE 3
Design and melting temperature of thermostable Abs

| Ab | Frameworks | Additional mutations | Tm (° C.) |
|---|---|---|---|
| D2-1A7 | IGVH3-30 | | 65 |
| | IGVλ3-19 | | |
| TS1 | IGVH3-23 | VH: E6Q, S49A | 71 |
| | IGVλ1-40 | Vλ: Q1S, V3E, G13V, R18T, | |
| | | S22T, L9K, F65S, K66S, A74T, | |
| | | L78A | |
| TS2 | IGVH3-23 | VH: E6Q, S49A | 65 |
| | IGVλ1-40 | Vλ: Q1S, S22T, F65S, K66G | |

CONCLUSIONS: TS1 exhibits a significant improvement in thermostability, with a six degree increase in Tₘ. This clone is appropriate for use, or for additional engineering to improve Protein A binding.

### Example 3—Identification of Region Preventing Protein a Binding

With the thermostability of the scFv improved to 71 degrees, Protein A affinity was next engineered into the Ab to facilitate manufacturing. VH3 frameworks are widely reported in the literature to be capable of binding Protein A. TS1 was engineered on a VH3-23 framework, but unexpectedly did not bind well to Protein A. Within the variable region of the heavy chain, residues within FR1, CDR2 and FR3 have been shown to be involved in Protein A binding. Structural analysis of TS1 revealed no amino acids that deviated from the germline at positions shown to be important for binding: H15, H17, H19, H57, H59, H64, H65, H66, H68, H70, H81, H82a, H82b. Given the germline sequence, it remained unclear which amino acids were negatively affecting the interaction between Protein A and the Ab, so a Protein A binding assay was developed.

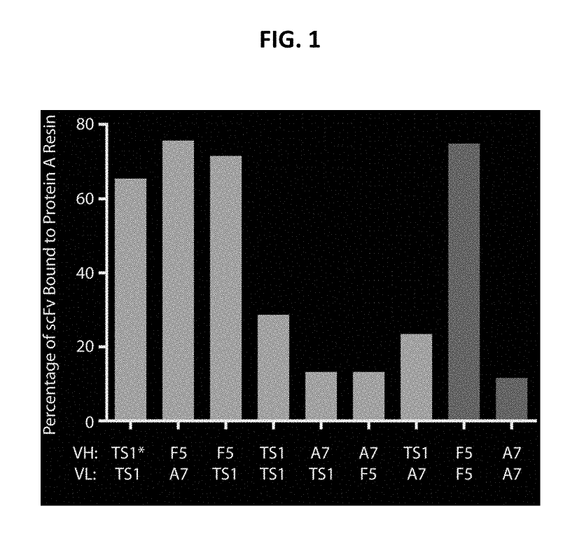

A set of variant scFvs was designed and prepared comprising heterologously paired heavy and light chains of D2-1A7, TS1, TS1* and F5. In order to directly test the effect of CDR-H2 sequence, an scFv was also made in which the CDR-H2 of TS1 was replaced with the CDR-H2 of F5 (TS1*). The variants were transiently expressed in mammalian cells, expressed scFv titers were quantitated using bio-layer interferometry (fortéBIO®). Protein A binding analysis was carried out in a 96 well plate as described in Example 1. The amount of scFv remaining in the flow-through was determined using bio-layer interferometry and the percentage of scFv that bound to the Protein A resin was calculated and the results were plotted (FIG. 1).

CONCLUSIONS: F5 exhibited 75% binding to Protein A. TS1 exhibited 29% Protein A binding, far below the 50% limit considered acceptable for commercial manufacture. The graft of the F5 CDR-H2 into TS1 (yielding scFv TS1*), while abrogating EphA2 binding, showed significant improvement in Protein A binding (65%), suggesting that the CDR-H2 of TS1 confers a negative effect on Protein A binding.

### Example 4—CDR-H2 Design and scFv Selection Using Yeast Display

A set of variants of the TS1CDRH2 was prepared for yeast display as described in Xu et al., (2013) mAbs, 5:2, 237-254; to screen for clones that retained EphA2 binding and exhibited improved Protein A binding. The set was designed to include over 50,000 distinct CDRH2 sequences. Each of the scFvs in the set has a FLAG™ epitope tag (DYKDDDDK—SEQ ID NO: 49) incorporated at its C-terminus. The set incorporated diversity at CDR-H2 positions suspected of being important for Protein A binding, as well as at CDR-H2 positions suspected of contributing to EphA2 binding. The design was then compared with databases of the human Ab repertoire downloaded from NCBI as well as NGS sequencing of non-immune patient repertoire and adjusted to preserve highly conserved residues. Ultimately, CDRH2 residues VH49, VH50, VH52A, VH53, VH55, and VH57 were identified for mutagenesis. Again guided by the human repertoire, only a subset of amino acids was selected for most positions (see Table 4). This resulted in the generation and testing of a set of scFvs designed to include 53,460 distinct sequences.

TABLE 4
scFv design: CDR-H2 mutations

| Position | TS1 | F5 | Residues Included in the Set |
|---|---|---|---|
| 49 | A | S | AGST (SEQ ID NO: 50) |
| 50 | V | A | AVDEG (SEQ ID NO: 51) |
| 51 | I | I | I |
| 52 | S | S | S |
| 52A | Y | G | CDGHNPRSTY (SEQ ID NO: 52) |
| 53 | D | R | ADGHNPRST (SEQ ID NO: 53) |
| 54 | G | G | G |
| 55 | S | D | ADGHNPRST (SEQ ID NO: 53) |
| 56 | N | N | N |
| 57 | K | T | TNK |
| 58 | Y | Y | Y |
| 59 | Y | Y | Y |
| 60 | A | A | A |

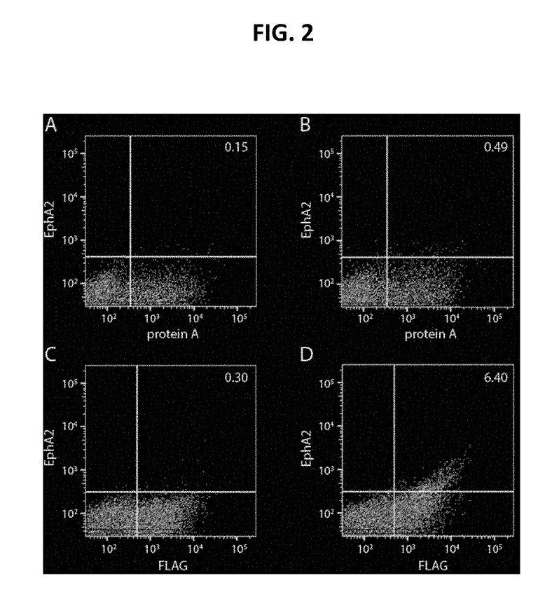

Rapid selection of the set of scFvs individually expressed on yeast cell surfaces to isolate those with desirable properties was carried out using FACS sorting of the yeast of on a Becton Dickenson® (BD) Aria® system. Induced yeast cells were incubated with 200 nM of EphA2 with an His6 tag and 10 μg/mL of fluorescently labeled Protein A. Cells were washed twice with FACS buffer (1×PBS, 0.5% BSA, pH 7.4) to remove unbound antigen. The antigen- and Protein A-bound cells were then incubated with 2 μg/mL Alexa647 labeled M2 anti-FLAG™ Ab and 2 μg/mL Alexa 488 labeled anti-His6 Ab for 30 minutes. After 2 washes, the cells were re-suspended in FACS buffer and the fluorescent signals were measured using a BD FACSCalibur® machine. The median fluorescent intensities (MFI) were determined using FlowJo® software. EphA2 MFI (anti-His6-Alexa 488) was normalized to expression MFI (anti-Flag-Alexa 647). For the first round of selection, selection for both EphA2 and Protein A binding was performed (FIG. 2, Panel B), which yielded a small percentage of the population. To ensure a population that bound to the recombinant EphA2 had been identified, the isolated clones were reselected for EphA2 binding (FIG. 2, Panel D).

CONCLUSIONS: Clones were obtained exhibiting desired characteristics.

### Example 5—Characterization of scFvs for Thermostability, EphA2 Binding/Internalization and Protein a Binding

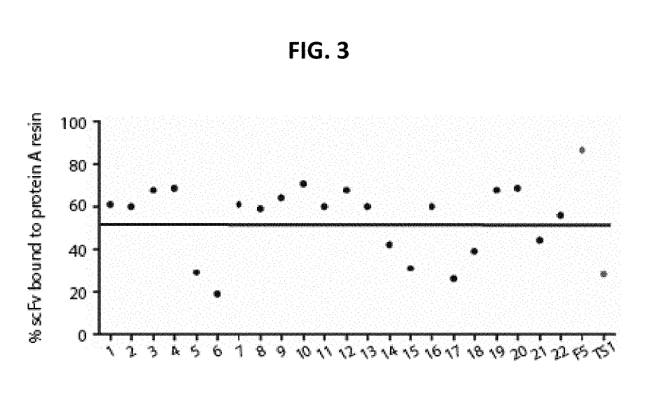

scFvs that were positive for Protein A and EphA2 binding when displayed on yeast were sequenced and transiently expressed as soluble proteins in Expi293F™ cells (Invitrogen®). They were then measured for Protein A binding as described in Example 1. 22 unique variants obtained as described in Example 4 were tested and exhibited Protein A binding ranging from 20% to 80% (FIG. 3). The amino acid and encoding nucleic acid sequences of these 22 scFvs (scFv1-scFv22) are provided in FIG. 5.

Of the 22 scFv tested, 15 resulted in at least 50% of the protein being bound to Protein A, and so these were selected for further characterization. The Tₘs of all variants were measured using DSF. All but four scFvs had desirable Tₘs, exceeding 65° C. by more than 2° C. (i.e., at least 67° C.). Eleven had even more favorable Tₘs of 270° C.

TABLE 5
scFvs identified from yeast display screening (and 2 controls)

| Ab | Tₘ (° C.) |
|---|---|
| TS1 | 72.5 |
| scFv-1 | 71.5 |
| scFv-2 | 70.7 |
| scFv-3 | 71.7 |
| scFv-4 | 69.3 |
| scFv-5 | 66.7 |
| scFv-6 | 68.7 |
| scFv-7 | 67.5 |
| scFv-8 | 70.3 |
| scFv-9 | 70.9 |
| scFv-10 | 72.9 |
| scFv-11 | 70.1 |
| scFv-12 | 75.7 |
| scFv-13 | 70.3 |
| scFv-14 | 61.9 |
| scFv-15 | 70.7 |
| scFv-16 | 69.5 |
| scFv-17 | 64.9 |
| scFv-18 | 72.5 |
| scFv-19 | 69.3 |
| scFv-20 | 68.3 |
| scFv-21 | 68.1 |
| scFv-22 | 62.3 |
| F5 | 78.9 |

Binding and Internalization Assays:

To test the anti-EphA2 activity of these variant scFvs, the binding and internalization of liposomes externally decorated with these Abs was assayed on cell lines expressing EphA2. A rapid high-throughput Chelated Ligand-induced Internalization Assay (CLIA) (Nielsen et al., BMC immunology, 2006, 7:24) was used to screen anti-EphA2-scFv-targeted liposomes for binding and internalization assessment. Two cell lines that express high levels of EphA2 receptors, OVCAR-3 and U-251, were tested, as well as a murine EphA2-expressing mouse cell line, CT-26 (to test whether cross-reactivity to murine EphA2 was retained by the engineered scFvs). Via nickel-binding by each scFv's c-terminal His6 tag, the scFvs were bound to fluorescently labeled liposomes comprising an NTA(Ni) conjugated lipid (Dil5-NTA liposomes).

Preparation of Test Liposomes and Internalization Assay

Dil5-NTA liposomes were prepared as follows. The NTA conjugated lipid, DOD-Tri-NTA, was synthesized as described in Huang et al., Bioconjugate chemistry, 2006, vol. 17, p. 1592-1600. Hydrogenated soy phosphatidylcholine (HSPC), cholesterol, methoxy-PEG(2000)-distearoylphosphatidylethanolamine (PEG-DSPE), DOD-tri-NTA, and a fluorescent lipid dye, Dil5, (ThermoFisher® Invitrogen® Molecular Probes™, cat. # D12370) were combined in a chloroform solution at the molar ratio of 100:66.7:5:0.5:0.3 respectively and the solution evaporated under vacuum to dryness forming a lipid film. The film was hydrated in HEPES-buffered physiological saline (“HBS-6.5”—5 mM HEPES, 144 mM NaCl pH 6.5) at 68° C., the resulting lipid suspension was subjected to several cycles of freezing (dry ice-acetone) and thawing (68C), and extruded through two stacked polycarbonate membranes (Whatman Nuclepore, USA) with the pore size of 200 nm (6 times) and 100 nm (10 times) using Lipex™ thermobarrel extruder (Northern Lipids, Canada). This protocol typically produces unilamellar vesicles with the z-average size of 100-110 nm and polydispersity index less than 0.1. The extruded liposomes were chilled to room temperature, purified by gel-chromatography on a Sephadex® G-75 (GE Healthcare) column eluted with HBS-6.5, and then sterilized by passage through a 0.2-μm filter. The concentration of liposomal phospholipid was then determined after acid digestion using spectrophotometric phosphomolybdate method. Prior to incubation with cells the liposomes were diluted to 0.4 mM phospholipid in Hank's Balanced Salt Solution (HBSS), and NiSO₄ was added to 0.1 mM. The hexahistidine scFvs were diluted into cell culture medium (see below) to the concentration of 25 μg/mL of scFv and mixed with equal volume of the liposome solution (“fluorescent NTA-liposome/scFv mixture”).

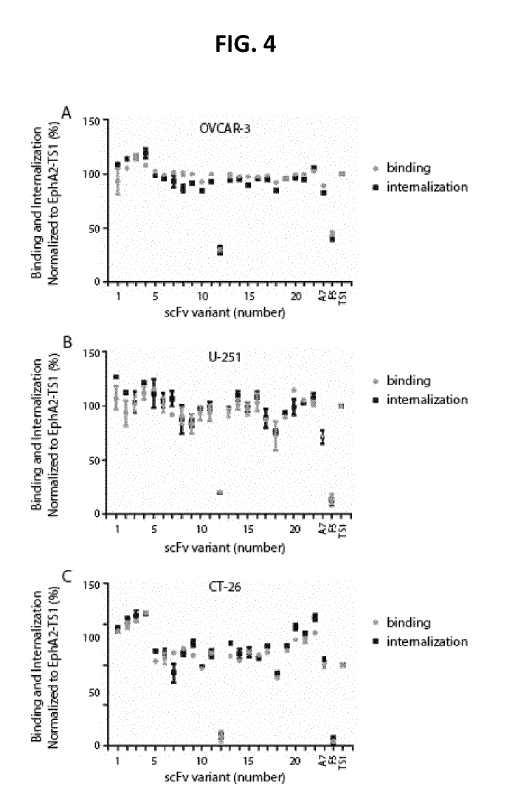

By measuring fluorescence of the cells prior to and after incubation/wash with imidazole, which disrupts His6/Ni complexes and thereby releases and removes external (but not internalized) histidine-tagged scFv, the percentage of Ab internalized is quantitated. The test cells (human cancer cell lines OVCAR-3 and U-251 and the murine cancer cell line CT-26) were grown in the adherent state in RPMI-640 cell culture medium supplemented with 10% FBS, 1× penicillin/streptomycin, and L-glutamine, to 90% confluence and harvested using 0.25% trypsin-EDTA. The cell suspension was dispensed at 100,000 cells/well into a 96-well “V” bottom shape polypropylene cell culture plate, the cells were washed with 1×PBS, and then re-suspended in 100 μL of fluorescent NTA-liposome/scFv mixture The plate was covered with plate sealing tape, protected from light, and incubated for 4 hours at 37° C. in the atmosphere of 5% CO₂ on a shaker. The cells were pelleted by centrifugation, the supernatants were aspirated, and the cells were washed twice by resuspension either in 200 μL/well of PBS to remove any unbound extracellular liposomes, or with 200 μL/well of PBS containing 0.25 M imidazole (pH 7.5) to remove unbound extracellular liposomes and surface-bound liposomes, but not internalized liposomes. The relative amounts of cell-associated liposomes were evaluated by FACS (Cy5 fluorescence channel). The “binding” amount included both cell surface-bound and internalized liposomes and was proportional to cell fluorescence without imidazole wash. It is shown in FIG. 4 as percent relative to the liposome binding by matched liposomes decorated with scFv TS1. The internalization data show the amount of internalized liposomes as percent of total cell associated liposomes. The majority of clones retained binding and internalization to human and murine EphA2 similar to TS1 (FIG. 4). Only one tested clone, scFv-12 (and the F5 negative control), did not bind EphA2.

CONCLUSIONS: 21 of the 22 new clones tested (all except scFv-12) were identified as having acceptable binding and internalization properties. 18 of the 22 (scFvs 1-4, 6-13, 15-16, and 18-21) exhibited desirable thermostability with a Tₘ of 267° C., while ten of these (scFvs 1-3, 8-11, 13, 15, and 18) also met a more commercially favorable thermostability criterion, each exhibiting a Tₘ of 270° C.

### Example 6—Testing Conjugation Stability of Anti-EphA2 scFvs

Selected scFvs were recombinantly modified at their C-termini to remove the His6 tags and add a C-terminal peptide—Gly Gly Ser Gly Gly Cys (SEQ ID NO: 54) adapted for liposome attachment. These modified scFvs are identified by names corresponding to those of the scFvs they were derived from followed by the letter C. The conjugation of the Abs (using methods described in U.S. Pat. No. 6,210,707) to PEG-DSPE (a lipopolymer), with the resulting lipopolymer-conjugated scFvs retaining the ability to bind antigen (measured as antigen-binding affinity), as well as the ability of the lipopolymer conjugates to insert (via the DSPE portion of the lipopolymer) into the outer membranes of liposomes (leaving PEG-tethered scFv externally exposed), efficiently and without loss of binding properties (measured as total affinity of liposome binding to antigen via multiple bound scFvs and referred to below as avidity) was next tested.

Conjugation of scFvs to a Lipopolymer Linker:

Purified scFv solutions were treated with 15 mM L-cysteine, 5 mM EDTA, adjusted to pH 6.0-6.2, at 30° C. for 1 hour to reduce/activate the thiol group of carboxy-terminal cysteine residue of each scFv. Excess cysteine was removed by gel-chromatography on a Sephadex® column eluted with conjugation buffer (5 mM citrate, 1 mM EDTA, 140 mM NaCl, pH 6.0). Next, the Abs were conjugated to the lipopolymer linker, maleimido-PEG(2000)-DSPE (mal-PEG-DSPE, Avanti Polar Lipids, cat. #880126), as follows. Mal-PEG-DSPE (Avanti) in the form of aqueous micellar stock solution was added to the treated scFvs to achieve lipid/protein molar ratio of 4:1. The mixture was incubated with stirring for 2-4 hours at room temperature and the reaction was stopped by quenching non-reacted maleimides with cysteine at a final concentration of 0.5 mM for five minutes. The quenched reaction mix was chromatographed on an Ultrogel AcA34 gravity column and eluted with S10C-6.5 buffer (100 g/L low endotoxin sucrose, 10 mM citric acid USP, adjusted to pH 6.5 with NaOH) with eluent protein concentration read at 280 nm. The first (void volume) protein peak, containing purified scFv-PEG-DSPE conjugate, was collected. The purity of scFv-PEG-DSPE conjugates was assessed by SDS polyacrylamide gel electrophoresis (SDS-PAGE).

Preparation of Test Liposomes and Insertion of scFv Lipopolymer Conjugates into Liposome Membrane:

Egg sphingomyelin (ESM, NOF, Japan, NM-10), cholesterol (Avant Polar Lipids, #770100), methoxy-PEG(2000)-distearoylglycerol (PEG-DSG, NOF, Japan, Sunbright GS-020), and Dil5 (see Example 5) at molar ratio 100:66.7:8:0.3, respectively, were dissolved at 70° C. in absolute ethanol and diluted with stirring at 70° C. into 10 volumes of CS-250 buffer (250 mM aqueous NaCl, 5 mM citrate, pH 5.5) to a final concentration of 100 mM phospholipid. The lipid suspension was extruded at 70° C. through 200 nm and 100 nm polycarbonate membrane filters as described above in Example 5 and the extruded material was cooled down to room temperature, the liposomes were purified by tangential flow diafiltration using a MiniKros® hollow fiber cartridge (Spectrum Laboratories, MWCO 500 kD) via 10 volume exchanges of CS-250 buffer, and passed through a 0.2-μm sterilizing filter. This protocol typically produces unilamellar vesicles with the z-average size of 100-110 nm and polydispersity index less than 0.1 (“Dil5-SM liposomes”). Before membrane insertion of the scFv-PEG-DSPE conjugates, the liposomes were exchanged into dextrose-citrate buffer (17% aqueous dextrose, 20 mM citrate, pH 5.7) using size exclusion chromatography (SEC) on a Sephadex® G-25 column (PD-10, GE Healthcare).

The scFv-PEG-DSPE conjugates were then mixed with Dil5-SM liposomes in dextrose-citrate buffer to achieve a protein/phospholipid ratio of 10-12 g/mol. The mixtures were quickly heated to 60° C. and maintained at this temperature for 30 minutes with stirring. Then the mixtures were chilled on ice and the liposomes with membrane-inserted scFv-PEG-DSPE conjugates were separated from any residual non-inserted conjugate by gel-chromatography on a column of Sepharose™ CL-4B (GE Healthcare) eluted with citrate saline buffer. The scFv-linked liposomes were collected in the void volume of the column and analyzed for in vitro antigen binding avidity using fortéBIO®, and for the cell uptake using cytofluorimetry, each as described below. The presence of scFv on the liposomes was confirmed and quantified by SDS-PAGE.

EphA2 Binding Analysis Via FortéBIO®

A nucleic acid encoding human EphA2 with c-terminal His6 appended was cloned into expression vector pCEP4 (Invitrogen® # V044-50) and transiently expressed in FreeStyle™ 293 cells (Invitrogen® #K900001) following polyethylenenimine transfection. The His6-tagged recombinant human EphA2 protein was purified from the cell culture medium using Ni-NTA Sepharose™ resin (Quiagen®, USA). Anti-His sensors for the fortéBIO® Octet Red 96 system (fortéBIO®-Pall® #18-5116) were coated with his-tagged recombinant, human EphA2 at concentration of 10 μg/ml protein for 300 sec, and a background run was carried out in PBS for 60 sec. For scFv-PEG-DSPE conjugates, the sensors were then incubated with 2.5 μg/ml (Study 1) or 4.0 μg/ml (Study 2) of the conjugate in PBS, pH 7.4, and the slopes of association curves were determined between 3-13 seconds of incubation, corrected for the buffer-only background, and compared across the variants. ScFv-linked liposomes were incubated with the sensors in PBS at 0.025 mM of liposome phospholipid, the slopes of the association curves were determined between 3-20 seconds of incubation, corrected for the buffer-only background, and compared across the variants.

Results are shown below in Table 6, in which column A gives scFv identifiers; column B indicates percent yield of scFv/lipopolymer linker conjugates; column C gives percent scFv-conjugate (not liposome-bound) EphA2 binding on-rate; column D gives percent scFv-conjugated-liposome EphA2 binding on-rate; and columns E (Study 1) and F (study 2) give percent antigen binding on-rate remaining in the scFv-conjugate (not liposome-bound) following thermal stress.

TABLE 6
Conjugation of selected scFv variants to lipopolymer linker and
EphA2-binding affinity/avidity of resulting conjugates/scFv-linked
liposomes.

| A | B | C | D | E | F |
|---|---|---|---|---|---|
| scFv-2C | 41.5 | 104.7 | 116.6 | 44.6 | 26.9 |
| scFv-3C | 48.5 | 112.5 | 119.1 | 56.5 | 46.9 |
| scFv-4C | 42.4 | 97.3 | 117.5 | 8.6 | 5.6 |
| scFv-7C | 50.6 | 109.9 | 119.2 | 5.8 | 4.8 |
| scFv-8C | 52.6 | 112.6 | 133.3 | 19.8 | 7.3 |
| scFv-9C | 53.4 | 114.4 | 118.1 | 25.2 | 12.2 |
| scFv-10C | 53 | 103.8 | 109.6 | 88.9 | 98.8 |
| scFv-11C | 52 | 109.2 | 120.5 | 1.6 | −0.09 |
| scFv-13C | 51.5 | 105.3 | 120.2 | 18.2 | 9.0 |
| scFv-16C | 65.2 | 128.5 | 113.4 | ND | 4.6 |
| scFv-19C | 57.2 | 111.7 | 129.3 | ND | 5.1 |
| TS1C | 76-78 | 100 | 100 | 25.2 | 13.0 |

CONCLUSIONS: All tested clones conjugated well, but scFvs 4C, 7C, 11C, 16C and 19C exhibited less than 10% antigen binding after thermal stress of conjugation.

### Example 7—Analysis of Selected Variants for Manufacturability

In parallel with the experiments of Example 6, nine clones (and parental clone TS1 as a control) were tested to determine suitability for commercial manufacture. A screening strategy was designed to eliminate molecules with low expression, low Protein A binding capacity, or high aggregate content. scFvs were transiently expressed at a 1 L scale in FreeStyle™ 293 cells (Invitrogen® #K900001), Protein A purified, and neutralized to pH 6.0. The results are summarized in Table 7.

TABLE 7
Characterization of developability at 1 L scale

| | | | | | Fold |
|---|---|---|---|---|---|
| | | | | | Improvement |
| | | Neutral- | Final Yield | Aggre- | in Protein A |
| | Titers | ization | (per 1 L of | gates | Binding |
| Ab | (mg/L) | Loss (%) | supernatant) | (%) | Capacity |
| scFv-2C | 12.4 | 21 | 9.8 | 7 | 5.5 |
| scFv-3C | 20.3 | 8 | 18.7 | 6 | 6 |
| scFv-4C | 13.1 | 7 | 12.2 | 7 | 5.8 |
| scFv-7C | 23.4 | 23 | 18.0 | 3 | 10.3 |
| scFv-8C | 19.3 | 15 | 16.4 | 6 | ND |
| scFv-9C | 19.3 | 15 | 16.4 | 4 | ND |
| scFv-10C | 27.7 | 17 | 23.0 | 11 | 12.3 |
| scFv-11C | 19.3 | 32 | 13.1 | 4 | 8.5 |
| scFv-13C | 21.0 | 8 | 19.3 | 4 | ND |
| TS1C | 11.8 | 15 | 10.0 | 11 | 1.0 |

All Ab titers were calculated using bio-layer interferometry (fortéBIO®). The percentage of aggregates was measured using SEC as follows. 50 μg of sample was injected on a TSKgel® SuperSW3000 column (Tosoh® Bioscience) using 50 mM sodium phosphate (+0.4 M sodium perchlorate) pH 7.0 as running buffer. All measurements were performed using an Agilent® 1100 HPLC equipped with an auto sampler, binary pump and diode array detector. Data were analyzed using Chemstation™ (Agilent®) software.

The fold improvement in Protein A binding capacity (as compared to TS1C) was measured as described in Example 1.

The first criterion evaluated was expression level of the scFvs. Antibodies are typically manufactured in stably transfected cell lines (e.g., in Chinese hamster ovary—“CHO” cells). However, titers obtained via transiently expression are predictive of expression behavior in stably transfected cells. As shown above in Table 7, many of the new Abs had much better titers (about a 2-fold or better improvement as compared to TS1C). The neutralization losses were a bit higher for some variants but all final purification yields were comparable or better as compared to TS1C. Finally, for successful downstream process development, it is desirable to have low aggregate content. The post neutralization aggregate content of all variants was comparable and acceptable. With the exception of scFv-2C and scFv-4C, these new Abs had much improved expression, aggregation, and Protein A binding capacity. All were further characterized for stability during the conjugation process.

CONCLUSIONS: All tested clones (clones 2, 3, 4, 7, 8, 9, 10, 11, and 13) exhibited acceptable commercial developability properties for tested parameters.

### EQUIVALENTS

Those skilled in the art will recognize, or be able to ascertain and implement using no more than routine experimentation, many equivalents of the specific embodiments described herein. Such equivalents are intended to be encompassed by the foregoing disclosure and the following claims. Any combinations of the embodiments disclosed in the particular embodiments (and in the dependent claims) are contemplated to be within the scope of the disclosure.

### INCORPORATION BY REFERENCE

The disclosure of each and every U.S. and foreign patent and pending patent application and publication referred to herein is specifically incorporated by reference herein in its entirety.

1. An anti-Ephrin type-A receptor 2 (EphA2) antibody comprising:
a VH comprising:
a VH CDR1 comprising the amino acid sequence SYAMH (SEQ ID NO: 17);
a VH CDR2 comprising
the following amino acid sequence of 17 amino acids:
X¹⁷ISPX¹⁸GX¹⁹NX²⁰YYADSVKG (SEQ ID NO: 18),
wherein:
X¹⁷ is A, or V;
X¹⁸ is A, D, H, N, P, S or T;
X¹⁹ is A, R, H, N or P; and
X²⁰ is K or T;
and
a VH CDR3 comprising the amino acid sequence ASVGATGPFDI (SEQ ID NO: 19); and
a VL comprising:
a VL CDR1 comprising the amino acid sequence QGDSLRSYYAS (SEQ ID NO: 20);
a VL CDR2 comprising the amino acid sequence GENNRPS (SEQ ID NO: 21); and
a VL CDR3 comprising the amino acid sequence NSRDSSGTHLTV (SEQ ID NO: 22);
wherein the antibody immunospecifically binds to the extracellular domain of EphA2.

2. A polypeptide that binds Protein A, wherein the polypeptide comprises the amino acid sequence:
a) X^{a}X¹⁷ISX^{b}X¹⁸GX¹⁹NX²⁰ (SEQ ID NO:198), wherein
X^{a} is S, T or A;
X¹⁷ is A, or V;
X^{b} is P, R, N or H,
X¹⁸ and X¹⁹ are any amino acid; and
X²⁰ is K or T;
b) X^{a}VISX^{b}X¹⁸GX¹⁹NX²⁰ (SEQ ID NO:199), wherein
X^{a} is S or T,
X^{b} is P, R, or N,
X¹⁸ is any amino acid;
X¹⁹ is H, N, R, T, A, D, P, or S; and
X²⁰ is K or T;
c) X^{a}VISX^{b}X¹⁸GX¹⁹NX²⁰ (SEQ ID NO:200), wherein
X^{a} is S or T,
X^{b} is P, R, or N,
X¹⁸ is any amino acid;
X¹⁹ is H, N, R, or T; and
X²⁰ is T or K;
or
d) X^{a}X¹⁷ISX^{b}X¹⁸GX¹⁹NX²⁰ (SEQ ID NO:201), wherein
X^{a} is S or T,
X¹⁷ is V;
X^{b} is P,
X¹⁸ is any amino acid;
X¹⁹ is H, N, R, or T; and
X²⁰ is T or K.

3. The polypeptide of claim 2, wherein the polypeptide comprises the amino acid sequence of one of SEQ ID NOS: 177-197.

4. The polypeptide of claim 2, wherein the polypeptide comprises:
a VH CDR1 comprising an amino acid sequence of SYAMH (SEQ ID NO:17);
a Protein A binding sequence comprising the amino acid sequence of one of SEQ ID NOs: 177 to 201; and
a VH CDR3 comprising an amino acid sequence of ASVGATGPFDI (SEQ ID NO: 19).

5. An antibody comprising a VH and a VL, wherein the VH comprises the polypeptide of claim 2.

6. The antibody of claim 1, wherein the antibody is human or humanized.

7. The antibody of claim 5, wherein the antibody is human or humanized.

8. The antibody of claim 1, wherein the antibody comprises at least one framework region selected from the group consisting of:
(a) a human VH framework region 1 (VH FR1) comprising the amino acid sequence QVQLVX¹SGGGLVQPGGSLRLSCAASGFTFS (SEQ ID NO: 1), wherein X¹ is an amino acid other than E,
(b) a human VH framework region 2 (VH FR2) comprising the amino acid sequence WVRQAPGKGLEWVX² (SEQ ID NO: 2), wherein X² is an amino acid other than S,
(c) a human VL framework region 1 (VL FR1) comprising the amino acid sequence X³SX⁴LTQPPSVSX⁵APGQX⁶VTIX⁷C (SEQ ID NO: 3), wherein: X³ is an amino acid other than Q, X⁴ is an amino acid other than V, X⁵ is an amino acid other than G, X⁶ is an amino acid other than R, and X⁷ is an amino acid other than S,
(d) a human VL framework region 1 (VL FR1) comprising the amino acid sequence X⁸SVLTQPPSVSGAPGQRVTIX⁹C (SEQ ID NO: 4), wherein: X⁸ is an amino acid other than Q, or X⁹ is an amino acid other than S,
(e) a human VL framework region 2 (VL FR2) comprising the amino acid sequence WYQQX¹⁰PGTAPKLLIY (SEQ ID NO: 5), wherein X¹⁰ is an amino acid other than L, and
(f) a human VL framework region 3 (VL FR3) comprising the amino acid sequence GVPDRFSGX¹¹X¹²SGTSASLX¹³ITGX¹⁴QAEDEADYYC (SEQ ID NO: 6), wherein: X¹¹ is an amino acid other than F, X¹² is an amino acid other than K, X¹³ is an amino acid other than A, or X¹⁴ is an amino acid other than L, and
(g) a human VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, or X¹⁶ is an amino acid other than K.

9. The antibody of claim 5, wherein the antibody comprises at least one framework region selected from the group consisting of:
(a) a human VH framework region 1 (VH FR1) comprising the amino acid sequence QVQLVX¹SGGGLVQPGGSLRLSCAASGFTFS (SEQ ID NO: 1), wherein X¹ is an amino acid other than E,
(b) a human VH framework region 2 (VH FR2) comprising the amino acid sequence WVRQAPGKGLEWV (SEQ ID NO: 202),
(c) a human VL framework region 1 (VL FR1) comprising the amino acid sequence X³SX⁴LTQPPSVSX⁵APGQX⁶VTIX⁷C (SEQ ID NO: 3), wherein: X³ is an amino acid other than Q, X⁴ is an amino acid other than V, X⁵ is an amino acid other than G, X⁶ is an amino acid other than R, and X⁷ is an amino acid other than S,
(d) a human VL framework region 1 (VL FR1) comprising the amino acid sequence X⁸SVLTQPPSVSGAPGQRVTIX⁹C (SEQ ID NO: 4), wherein: X⁸ is an amino acid other than Q, or X⁹ is an amino acid other than S,
(e) a human VL framework region 2 (VL FR2) comprising the amino acid sequence WYQQX¹⁰PGTAPKLLIY (SEQ ID NO: 5), wherein X¹⁰ is an amino acid other than L, and
(f) a human VL framework region 3 (VL FR3) comprising the amino acid sequence GVPDRFSGX¹¹X¹²SGTSASLX¹³ITGX¹⁴QAEDEADYYC (SEQ ID NO: 6), wherein: X¹¹ is an amino acid other than F, X¹² is an amino acid other than K, X¹³ is an amino acid other than A, or X¹⁴ is an amino acid other than L, and
(g) a human VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, or X¹⁶ is an amino acid other than K.

10. The antibody of claim 1, wherein the antibody comprises a VH FR1 comprising the amino acid sequence QVQLVQSGGGLVQPGGSLRLSCAASGFTFS (SEQ ID NO: 8).

11. The antibody of claim 1, wherein the VH FR2 comprises the amino acid sequence WVRQAPGKGLEWVA (SEQ ID NO: 9) or WVRQAPGKGLEWVT (SEQ ID NO: 10).

12. The antibody of claim 1, wherein the antibody comprises the VL FR1 comprising the amino acid sequence X³SX⁴LTQPPSVSX⁵APGQX⁶VTIX⁷C (SEQ ID NO: 3), and wherein X³ is an amino acid other than Q, X⁴ is an amino acid other than V, X⁵ is an amino acid other than G, X⁶ is an amino acid other than R, and X⁷ is an amino acid other than S.

13. The antibody of claim 1, wherein the antibody comprises a VL FR1 comprising the amino acid sequence X³SX⁴LTQPPSVSX⁵APGQX⁶VTIX⁷C (SEQ ID NO: 3), and wherein X³ is S, X⁴ is E, X⁵ is V, X⁶ is T, and X⁷ is T.

14. The antibody of claim 1, wherein the antibody comprises a VL FR1 comprising the amino acid sequence X⁸SVLTQPPSVSGAPGQRVTIX⁹C (SEQ ID NO: 4), and wherein X⁸ is an amino acid other than Q, and X⁹ is an amino acid other than S.

15. The antibody of claim 1, wherein the antibody comprises a VL FR1 comprising the amino acid sequence X⁸SVLTQPPSVSGAPGQRVTIX⁹C (SEQ ID NO: 4), and wherein X⁸ is S or X⁹ is T.

16. The antibody of claim 1, wherein the antibody comprises a VL FR1 comprising the amino acid sequence X⁸SVLTQPPSVSGAPGQRVTIX⁹C (SEQ ID NO: 4), and wherein X⁸ is S and X⁹ is T.

17. The antibody of claim 1, wherein the antibody comprises a VL FR2 comprising the amino acid sequence WYQQX¹⁰PGTAPKLLIY (SEQ ID NO: 5), and wherein X¹⁰ is an amino acid other than L.

18. The antibody of claim 1, wherein the antibody comprises a VL FR2 comprising the amino acid sequence WYQQX¹⁰PGTAPKLLIY (SEQ ID NO: 5), and wherein X¹⁰ is K.

19. The antibody of claim 1, wherein the antibody comprises a VL FR3 comprising the amino acid sequence GVPDRFSGX¹¹X¹²SGTSASLX¹³ITGX¹⁴QAEDEADYYC (SEQ ID NO: 6), and wherein X¹¹ is an amino acid other than F, X¹² is an amino acid other than K, X¹³ is an amino acid other than A, and X¹⁴ is an amino acid other than L.

20. The antibody of claim 1, wherein the antibody comprises a VL FR3 comprising the amino acid sequence GVPDRFSGX¹¹X¹²SGTSASLX¹³ITGX¹⁴QAEDEADYYC, (SEQ ID NO: 6) and wherein X¹¹ is S, X¹² is S, X¹³ is T, or X¹⁴ is A.

21. The antibody of claim 1, wherein the antibody comprises a VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, and X¹⁶ is an amino acid other than K.

22. The antibody of claim 1, wherein the antibody comprises a VL FR3 comprising the amino acid sequence GVPDRFSGX¹⁵X¹⁶SGTSASLAITGLQAEDEADYYC (SEQ ID NO: 7), wherein: X¹⁵ is S or X¹⁶ is G.

23. The antibody of claim 1, wherein the antibody comprises a VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is S and X¹⁶ is G.

24. The antibody of claim 1, comprising:
a VH framework region 1 (VH FR1) comprising the amino acid sequence QVQLVX¹SGGGLVQPGGSLRLSCAASGFTFS, (SEQ ID NO: 1) wherein X¹ is an amino acid other than E;
a VH framework region 2 (VH FR2) comprising the amino acid sequence WVRQAPGKGLEWVX² (SEQ ID NO: 2), wherein X² is an amino acid other than S;
a VL framework region 1 (VL FR1) comprising the amino acid sequence X³SX⁴LTQPPSVSX⁵APGQX⁶VTIX⁷C (SEQ ID NO: 3), wherein X³ is an amino acid other than Q, X⁴ is an amino acid other than V, X⁵ is an amino acid other than G, X⁶ is an amino acid other than R, and X⁷ is an amino acid other than S;
a VL framework region 2 (VL FR2) comprising the amino acid sequence WYQQX¹⁰PGTAPKLLIY (SEQ ID NO: 5), wherein X¹⁰ is an amino acid other than L; and
a VL framework region 3 (VL FR3) comprising the amino acid sequence GVPDRFSGX¹¹X¹²SGTSASLX¹³ITGX¹⁴QAEDEADYYC (SEQ ID NO: 6), wherein X¹¹ is an amino acid other than F, X¹² is an amino acid other than K, X¹³ is an amino acid other than A, and X¹⁴ is an amino acid other than L.

25. The antibody of claim 1, comprising:
a VH framework region 1 (VH FR1) comprising the amino acid sequence QVQLVX¹SGGGLVQPGGSLRLSCAASGFTFS (SEQ ID NO: 1), wherein X¹ is an amino acid other than E;
a VH framework region 2 (VH FR2) comprising the amino acid sequence WVRQAPGKGLEWVX² (SEQ ID NO: 2), wherein X² is an amino acid other than S;
a VL framework region 1 (VL FR1) comprising the amino acid sequence X⁸SVLTQPPSVSGAPGQRVTIX⁹C (SEQ ID NO: 4), wherein: X⁸ is an amino acid other than Q, or X⁹ is an amino acid other than S; and
a VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, and X¹⁶ is an amino acid other than K.

26. The antibody of claim 5, comprising:
a VH framework region 1 (VH FR1) comprising the amino acid sequence QVQLVX¹SGGGLVQPGGSLRLSCAASGFTFS, (SEQ ID NO: 1) wherein X¹ is an amino acid other than E;
a VH framework region 2 (VH FR2) comprising the amino acid sequence WVRQAPGKGLEWVX² (SEQ ID NO: 2), wherein X² is an amino acid other than S;
a VL framework region 1 (VL FR1) comprising the amino acid sequence X³SX⁴LTQPPSVSX⁵APGQX⁶VTIX⁷C (SEQ ID NO: 3), wherein X³ is an amino acid other than Q, X⁴ is an amino acid other than V, X⁵ is an amino acid other than G, X⁶ is an amino acid other than R, and X⁷ is an amino acid other than S;
a VL framework region 2 (VL FR2) comprising the amino acid sequence WYQQX¹⁰PGTAPKLLIY (SEQ ID NO: 5), wherein X¹⁰ is an amino acid other than L; and
a VL framework region 3 (VL FR3) comprising the amino acid sequence GVPDRFSGX¹¹X¹²SGTSASLX¹³ITGX¹⁴QAEDEADYYC (SEQ ID NO: 6), wherein X¹¹ is an amino acid other than F, X¹² is an amino acid other than K, X¹³ is an amino acid other than A, and X¹⁴ is an amino acid other than L.

27. The antibody of claim 5, comprising:
a VH framework region 1 (VH FR1) comprising the amino acid sequence QVQLVX¹SGGGLVQPGGSLRLSCAASGFTFS (SEQ ID NO: 1), wherein X¹ is an amino acid other than E;
a VH framework region 2 (VH FR2) comprising the amino acid sequence WVRQAPGKGLEWVX² (SEQ ID NO: 2), wherein X² is an amino acid other than S;
a VL framework region 1 (VL FR1) comprising the amino acid sequence X⁸SVLTQPPSVSGAPGQRVTIX⁹C (SEQ ID NO: 4), wherein: X⁸ is an amino acid other than Q, or X⁹ is an amino acid other than S; and
a VL framework region 3 (VL FR3) comprising the amino acid sequence GVPDRFSGX¹⁵X¹⁶SGTSASLAITGLQAEDEADYYC (SEQ ID NO: 7), wherein: X¹⁵ is an amino acid other than F, and X¹⁶ is an amino acid other than K.

28. The antibody of claim 1, wherein the antibody has a melting temperature (Tₘ) of 67° C. or higher as measured using differential scanning fluorimetry.

29. The antibody of claim 1, wherein the antibody is a human antibody.

30. The antibody of claim 1, wherein the antibody is a single chain Fv (scFv), IgG, Fab, (Fab)₂, or (scFv′)₂.

31. The antibody of claim 1, wherein the antibody is an scFv.

32. The antibody of claim 1, wherein the X¹⁷ of the VH CDR2 is V; X¹⁸ is H or D; X¹⁹ is H or N; and X²⁰ is T.

33. The antibody of claim 1, wherein the VH CDR2 comprises an amino acid sequence selected from:
(SEQ ID NO: 23)
a. VISPAGNNTYYADSVKG,
(SEQ ID NO: 24)
b. VISPAGRNKYYADSVKG,
(SEQ ID NO: 25)
c. VISPDGHNTYYADSVKG,
(SEQ ID NO: 26)
d. VISPHGRNKYYADSVKG,
(SEQ ID NO: 27)
e. VISRRGDNKYYADSVKG,
(SEQ ID NO: 28)
f. VISNNGHNKYYADSVKG,
(SEQ ID NO: 29)
g. VISPAGPNTYYADSVKG,
(SEQ ID NO: 30)
h. VISPSGHNTYYADSVKG,
(SEQ ID NO: 31)
i. VISPNGHNTYYADSVKG,
(SEQ ID NO: 32)
j. AISPPGHNTYYADSVKG,
(SEQ ID NO: 33)
k. VISPTGANTYYADSVKG,
(SEQ ID NO: 34)
l. VISPHGSNKYYADSVKG,
(SEQ ID NO: 35)
m. VISNNGHNTYYADSVKG,
(SEQ ID NO: 36)
n. VISPAGTNTYYADSVKG,
(SEQ ID NO: 37)
o. VISPPGHNTYYADSVKG,
(SEQ ID NO: 38)
p. VISHDGTNTYYADSVKG,
(SEQ ID NO: 39)
q. VISRHGNNKYYADSVKG,
and
(SEQ ID NO: 40)
r. VISYDGSNKYYADSVKG.

34. An antibody comprising
a VH sequence comprising in amino- to carboxy-terminal order:
(i) VH FR1 sequence QVQLVQSGGGLVQPGGSLRLSCAASGFTFS (SEQ ID NO: 1),
(ii) VH CDR1 SYAMH (SEQ ID NO: 10),
(iii) either VH FR2 sequence WVRQAPGKGLEWVT (SEQ ID NO: 3) or
VH FR2 sequence WVRQAPGKGLEWVA (SEQ ID NO: 2),
(iv) VH CDR2 sequence X¹⁷ISPX¹⁸GX¹⁹NX²⁰YYADSVKG (SEQ ID NO: 11),
(v) VH FR3 sequence RFTISRDNSKNTLYLQMNSLRAEDTAVYYCAR (SEQ ID NO: 4),
(vi) VH CDR3 sequence ASVGATGPFDI (SEQ ID NO: 12),
(vii) VH FR4 sequence WGQGTLVTVSS (SEQ ID NO: 5),
and
a VL sequence comprising in amino- to carboxy-terminal order:
(viii) VL FR1 sequence SSELTQPPSVSVAPGQTVTITC (SEQ ID NO: 6),
(ix) VL CDR1 sequence QGDSLRSYYAS (SEQ ID NO: 13),
(x) VL FR2 sequence WYQQKPGTAPKLLIY (SEQ ID NO: 7),
(xi) VL CDR2 sequence GENNRPS (SEQ ID NO: 14),
(xii) VL FR3 sequence GVPDRFSGSSSGTSASLTITGAQAEDEADYYC (SEQ ID NO: 8),
(xiii) VL CDR3 sequence NSRDSSGTHLTV (SEQ ID NO: 15),
(xiv) VL FR4 sequence FGGGTKLTVLG (SEQ ID NO: 9),
and wherein,
X¹⁷ is A or V; X¹⁸ is A, D, H, N, P, S or T; X¹⁹ is A, R, H or P; and X²⁰ is K or T.

35. The antibody of claim 34, wherein the VH CDR2 comprises an amino acid sequence selected from:
(SEQ ID NO: 23)
a. VISPAGNNTYYADSVKG,
(SEQ ID NO: 24)
b. VISPAGRNKYYADSVKG,
(SEQ ID NO: 25)
c. VISPDGHNTYYADSVKG,
(SEQ ID NO: 26)
d. VISPHGRNKYYADSVKG,
(SEQ ID NO: 27)
e. VISRRGDNKYYADSVKG,
(SEQ ID NO: 28)
f. VISNNGHNKYYADSVKG,
(SEQ ID NO: 29)
g. VISPAGPNTYYADSVKG,
(SEQ ID NO: 30)
h. VISPSGHNTYYADSVKG,
(SEQ ID NO: 31)
i. VISPNGHNTYYADSVKG,
(SEQ ID NO: 32)
j. AISPPGHNTYYADSVKG,
(SEQ ID NO: 33)
k. VISPTGANTYYADSVKG,
(SEQ ID NO: 34)
l. VISPHGSNKYYADSVKG,
(SEQ ID NO: 35)
m. VISNNGHNTYYADSVKG,
(SEQ ID NO: 36)
n. VISPAGTNTYYADSVKG,
(SEQ ID NO: 37)
o. VISPPGHNTYYADSVKG,
(SEQ ID NO: 38)
p. VISHDGTNTYYADSVKG,
(SEQ ID NO: 39)
q. VISRHGNNKYYADSVKG,
and
(SEQ ID NO: 40)
r. VISYDGSNKYYADSVKG.

36. An antibody comprising a VH and a VL, wherein the VH and VL comprising an amino acid sequence at least 95% identical to:
the amino acid sequence of the VH and VL of SEQ ID NO: 60 [scFv1],
the amino acid sequence of the VH and VL of SEQ ID NO: 62 [scFv2],
the amino acid sequence of the VH and VL of SEQ ID NO: 64 [scFv3]
the amino acid sequence of the VH and VL of SEQ ID NO: 66 [scFv4],
the amino acid sequence of the VH and VL of SEQ ID NO: 68 [scFv5],
the amino acid sequence of the VH and VL of SEQ ID NO: 70 [scFv6],
the amino acid sequence of the VH and VL of SEQ ID NO: 72 [scFv7].
the amino acid sequence of the VH and VL of SEQ ID NO: 74 [scFv8],
the amino acid sequence of the VH and VL of SEQ ID NO: 76 [scFv9],
the amino acid sequence of the VH and VL of SEQ ID NO: 78 [scFv1 0],
the amino acid sequence of the VH and VL of SEQ ID NO: 80 [scFv1 1],
the amino acid sequence of the VH and VL of SEQ ID NO: 82 [scFv12],
the amino acid sequence of the VH and VL of SEQ ID NO: 84 [scFv13],
the amino acid sequence of the VH and VL of SEQ ID NO: 86 [scFv14],
the amino acid sequence of the VH and VL of SEQ ID NO: 88 [scFv15],
the amino acid sequence of the VH and VL of SEQ ID NO: 90 [scFv16],
the amino acid sequence of the VH and VL of SEQ ID NO: 92 [scFv17],
the amino acid sequence of the VH and VL of SEQ ID NO: 94 [scFv18],
the amino acid sequence of the VH and VL of SEQ ID NO: 96 [scFv19],
the amino acid sequence of the VH and VL of SEQ ID NO: 98 [scFv20],
the amino acid sequence of the VH and VL of SEQ ID NO: 100 [scFv21], or
the amino acid sequence of the VH and VL of SEQ ID NO: 102 [scFv22].

37. An antibody that is an scFv comprising:
a VH comprising:
a VH CDR1 having amino acid sequence SYAMH (SEQ ID NO: 17);
a VH CDR2 having one of the following 18 amino acid sequences:

| (SEQ ID NO: 23) | |
|---|---|
| (I) | VISPAGNNTYYADSVKG, |
| (SEQ ID NO: 24) | |
| (II) | VISPAGRNKYYADSVKG, |
| (SEQ ID NO: 25) | |
| (III) | VISPDGHNTYYADSVKG, |
| (SEQ ID NO: 26) | |
| (IV) | VISPHGRNKYYADSVKG, |
| (SEQ ID NO: 27) | |
| (V) | VISRRGDNKYYADSVKG, |
| (SEQ ID NO: 28) | |
| (VI) | VISNNGHNKYYADSVKG, |
| (SEQ ID NO: 29) | |
| (VII) | VISPAGPNTYYADSVKG, |
| (SEQ ID NO: 30) | |
| (VIII) | VISPSGHNTYYADSVKG, |
| (SEQ ID NO: 31) | |
| (IX) | VISPNGHNTYYADSVKG, |
| (SEQ ID NO: 32) | |
| (X) | AISPPGHNTYYADSVKG, |
| (SEQ ID NO: 33) | |
| (XI) | VISPTGANTYYADSVKG, |
| (SEQ ID NO: 34) | |
| (XII) | VISPHGSNKYYADSVKG, |
| (SEQ ID NO: 35) | |
| (XIII) | VISNNGHNTYYADSVKG, |
| (SEQ ID NO: 36) | |
| (XIV) | VISPAGTNTYYADSVKG, |
| (SEQ ID NO: 37) | |
| (XV) | VISPPGHNTYYADSVKG, |
| (SEQ ID NO: 38) | |
| (XVI) | VISHDGTNTYYADSVKG, |
| (SEQ ID NO: 39) | |
| (xvii) | VISRHGNNKYYADSVKG, |
| or | |
| (SEQ ID NO: 40) | |
| (xviii) | VISYDGSNKYYADSVKG; |

and
a VH CDR3 having amino acid sequence ASVGATGPFDI (SEQ ID NO: 19);
wherein the VH CDRs are present in the following amino to carboxyl terminal order: VH CDR1, VH CDR2, VH CDR3,
and the scFv further comprising:
a VL comprising:
a VL CDR1 having amino acid sequence QGDSLRSYYAS (SEQ ID NO: 20),
a VL CDR2 having amino acid sequence GENNRPS (SEQ ID NO: 21), and
a VL CDR3 having amino acid sequence NSRDSSGTHLTV (SEQ ID NO: 22);
and wherein
the VL CDRs are present in the following amino to carboxyl terminal order: VL CDR1, VL CDR2, and VL CDR3;
the scFv further comprising human frameworks:

| (SEQ ID NO: 8) | |
|---|---|
| VH FR1 | QVQLVQSGGGLVQPGGSLRLSCAASGFTFS |
| (SEQ ID NO: 9) | |
| VH FR2 | WVRQAPGKGLEWVA |
| (SEQ ID NO: 11) | |
| VH FR3 | RFTISRDNSKNTLYLQMNSLRAEDTAVYYCAR |
| (SEQ ID NO: 12) | |
| VH FR4 | WGQGTLVTVSS |
| (SEQ ID NO: 13) | |
| VL FR1 | SSELTQPPSVSVAPGQTVTITC |
| (SEQ ID NO: 14) | |
| VL FR2 | WYQQKPGTAPKLLIY |
| (SEQ ID NO:15) | |
| VL FR3 | GVPDRFSGSSSGTSASLTITGAQAEDEADYYC |
| and | |
| (SEQ ID NO: 16) | |
| VL FR4 | FGGGTKLTVLG |

and wherein the antibody immunospecifically binds to the extracellular domain of ephrin type-A receptor 2 (EphA2).

38. The antibody that is an scFv of claim 37 wherein the VH is amino terminal to the VL.

39. The antibody that is an scFv of claim 37, wherein the scFv exhibits a Tₘ of at least about 67° C.

40. The antibody that is an scFv of claim 37, wherein the scFv exhibits a Tₘ of at least about 70° C.

41. The antibody of claim 1 wherein the VH is amino terminal to the VL and joined to the VL by a polypeptide linker of from 10 to 30 amino acids in length.

42. The antibody of claim 1 wherein the linker comprises an amino acid sequence selected from:
(SEQ ID NO: 41)
ASTGGGGSGGGGSGGGGSGGGGS,
(SEQ ID NO: 42)
GGGGSGGGGSGGGGSGGGGS,
(SEQ ID NO: 43)
GGGGSGGGGSGGGGS,
(SEQ ID NO: 44)
ASTGGGGAGGGGAGGGGAGGGGA,
(SEQ ID NO: 45)
GGGGAGGGGAGGGGAGGGGA,
(SEQ ID NO: 46)
TPSHNSHQVPSAGGPTANSGTSGS,
and
(SEQ ID NO: 47)
GGSSRSSSSGGGGSGGGG.

43. The antibody of claim 42, wherein the scFv linker comprises the sequence ASTGGGGSGGGGSGGGGSGGGGS (SEQ ID NO: 41).

44. An antibody comprising a VH and a VL, wherein the VH comprises an amino acid sequence having at least 95% amino acid sequence identity to the amino acid sequence of SEQ ID NO: 133, 135, 137, 139, 141, 143, 145, 147, 149, 151, 153, 155, 157, 159, 161, 163, 165, 167, 169, 171, 173, or 175.

45. The antibody of claim 44, herein the VL comprises an amino acid sequence having at least 95% amino acid sequence identity to the amino acid sequence of SEQ ID NO: 134, 136, 138, 140, 142, 144, 146, 148, 150, 152, 154, 156, 158, 160, 162, 164, 166, 168, 170, 172, 174, or 176.

46. An antibody that is an scFv comprising an amino acid sequence at least 95% identical to the amino acid sequence of
SEQ ID NO: 60 [scFv1],
SEQ ID NO: 62 [scFv2],
SEQ ID NO: 64 [scFv3],
SEQ ID NO:66 [scFv4],
SEQ ID NO: 68 [scFv5],
SEQ ID NO: 70 [scFv6],
SEQ ID NO: 72 [scFv7].
SEQ ID NO: 74 [scFv8],
SEQ ID NO: 76 [scFv9],
SEQ ID NO: 78 [scFv10],
SEQ ID NO: 80 [scFv11],
SEQ ID NO: 82 [scFv12],
SEQ ID NO: 84 [scFv13],
SEQ ID NO: 86 [scFv14],
SEQ ID NO: 88 [scFv15],
SEQ ID NO: 90 [scFv16],
SEQ ID NO: 92 [scFv17],
SEQ ID NO: 94 [scFv18],
SEQ ID NO: 96 [scFv19],
SEQ ID NO: 98 [scFv20],
SEQ ID NO: 100 [scFv21], or
SEQ ID NO: 102 [scFv22].

47. The antibody or the scFv of claim 1, further comprising, at the C-terminus, Gly Gly Ser Gly Gly Cys (SEQ ID NO: 54).

48. An antibody conjugate comprising the antibody or the scFv of claim 1; and a moiety conjugated to the antibody.

49. The conjugate of claim 48, wherein the moiety is a lipidic nanoparticle.

50. The conjugate of claim 49, wherein the lipidic nanoparticle is a liposome.

51. The conjugate of claim 48, wherein the moiety comprises polyethylene glycol (PEG).

52. The conjugate of claim 48, wherein the moiety comprises a therapeutic agent.

53. The conjugate of claim 52, wherein the therapeutic agent is a cytotoxic agent.

54. The conjugate of claim 48, wherein the moiety comprises a detectable label.

55. The conjugate of claim 54, wherein the detectable label is an in vivo imaging agent.

56. A composition, comprising:
the antibody, the scFv, or the antibody conjugate of claim 1; and
a pharmaceutically acceptable carrier.

57. The composition of claim 56, wherein the composition is formulated for parenteral administration.

58. A kit comprising the composition of claim 56 in a container.

59. A nucleic acid encoding the antibody of claim 1.

60. An isolated nucleic acid encoding a variable light chain polypeptide, a variable heavy chain polypeptide, or both, of the antibody of claim 1.

61. An expression vector comprising:
the nucleic acid of claim 59; and
a eukaryotic promoter operably linked to the nucleic acid.

62. A host cell comprising the expression vector of claim 61.

63. The host cell of claim 62, wherein the cell expresses the variable light chain polypeptide, the variable heavy chain polypeptide, or both.

64. A method of treating a subject having an EphA2-expressing cancer, the method comprising:
administering to a subject having an EphA2-expressing cancer the antibody of claim 1.

65.-66. (canceled)


SEQUENCE LISTING (202 sequences)

SEQ ID NO: 1 - protein, 30 aa, Artificial sequence
  Synthetic polypeptide.
  MISC_FEATURE (6)..(6): Xaa may be any amino acid except Glu.
QVQLVXSGGGLVQPGGSLRLSCAASGFTFS

SEQ ID NO: 2 - protein, 14 aa, Artificial sequence
  Synthetic polypeptide.
  MISC_FEATURE (14)..(14): Xaa may be any amino acid except Ser.
WVRQAPGKGLEWVX

SEQ ID NO: 3 - protein, 22 aa, Artificial sequence
  Synthetic polypeptide.
  MISC_FEATURE (1)..(1): Xaa may be any amino acid except Gln.
  MISC_FEATURE (3)..(3): Xaa may be any amino acid except Val.
  MISC_FEATURE (12)..(12): Xaa may be any amino acid except Gly.
  MISC_FEATURE (17)..(17): Xaa may be any amino acid except Arg.
  MISC_FEATURE (21)..(21): Xaa may be any amino acid except Ser.
XSXLTQPPSVSXAPGQXVTIXC

SEQ ID NO: 4 - protein, 22 aa, Artificial sequence
  Synthetic polypeptide.
  MISC_FEATURE (1)..(1): Xaa may be any amino acid except Gln.
  MISC_FEATURE (21)..(21): Xaa may be any amino acid except Ser.
XSVLTQPPSVSGAPGQRVTIXC

SEQ ID NO: 5 - protein, 15 aa, Artificial sequence
  Synthetic polypeptide.
  MISC_FEATURE (5)..(5): Xaa may be any amino acid except Leu.
WYQQXPGTAPKLLIY

SEQ ID NO: 6 - protein, 32 aa, Artificial sequence
  Synthetic polypeptide.
  MISC_FEATURE (9)..(9): Xaa may be any amino acid except Phe.
  MISC_FEATURE (10)..(10): Xaa may be any amino acid except Lys.
  MISC_FEATURE (18)..(18): Xaa may be any amino acid except Ala.
  MISC_FEATURE (22)..(22): Xaa may be any amino acid except Leu.
GVPDRFSGXXSGTSASLXITGXQAEDEADYYC

SEQ ID NO: 7 - protein, 32 aa, Artificial sequence
  Synthetic polypeptide.
  MISC_FEATURE (9)..(9): Xaa may be any amino acid except Phe.
  MISC_FEATURE (10)..(10): Xaa may be any amino acid except Lys.
GVPDRFSGXXSGTSASLAITGLQAEDEADYYC

SEQ ID NO: 8 - protein, 30 aa, Artificial sequence
  Synthetic polypeptide.
QVQLVQSGGGLVQPGGSLRLSCAASGFTFS

SEQ ID NO: 9 - protein, 14 aa, Artificial sequence
  Synthetic polypeptide.
WVRQAPGKGLEWVA

SEQ ID NO: 10 - protein, 14 aa, Artificial sequence
  Synthetic polypeptide.
WVRQAPGKGLEWVT

SEQ ID NO: 11 - protein, 32 aa, Artificial sequence
  Synthetic polypeptide.
RFTISRDNSKNTLYLQMNSLRAEDTAVYYCAR

SEQ ID NO: 12 - protein, 11 aa, Artificial sequence
  Synthetic polypeptide.
WGQGTLVTVSS

SEQ ID NO: 13 - protein, 22 aa, Artificial sequence
  Synthetic polypeptide.
SSELTQPPSVSVAPGQTVTITC

SEQ ID NO: 14 - protein, 15 aa, Artificial sequence
  Synthetic polypeptide.
WYQQKPGTAPKLLIY

SEQ ID NO: 15 - protein, 32 aa, Artificial sequence
  Synthetic polypeptide.
GVPDRFSGSSSGTSASLTITGAQAEDEADYYC

SEQ ID NO: 16 - protein, 11 aa, Artificial sequence
  Synthetic polypeptide.
FGGGTKLTVLG

SEQ ID NO: 17 - protein, 5 aa, Artificial sequence
  Synthetic polypeptide.
SYAMH

SEQ ID NO: 18 - protein, 17 aa, Artificial sequence
  Synthetic polypeptide.
  MISC_FEATURE (1)..(1): Xaa may be Ala or Val.
  MISC_FEATURE (5)..(5): Xaa may be Ala, Asp, His, Asn, Pro, Ser or Thr.
  MISC_FEATURE (7)..(7): Xaa may be Ala, Arg, His or Pro.
  MISC_FEATURE (9)..(9): Xaa may be Lys or Thr.
XISPXGXNXYYADSVKG

SEQ ID NO: 19 - protein, 11 aa, Artificial sequence
  Synthetic polypeptide.
ASVGATGPFDI

SEQ ID NO: 20 - protein, 11 aa, Artificial sequence
  Synthetic polypeptide.
QGDSLRSYYAS

SEQ ID NO: 21 - protein, 7 aa, Artificial sequence
  Synthetic polypeptide.
GENNRPS

SEQ ID NO: 22 - protein, 12 aa, Artificial sequence
  Synthetic polypeptide.
NSRDSSGTHLTV

SEQ ID NO: 23 - protein, 17 aa, Artificial sequence
  Synthetic polypeptide.
VISPAGNNTYYADSVKG

SEQ ID NO: 24 - protein, 17 aa, Artificial sequence
  Synthetic polypeptide.
VISPAGRNKYYADSVKG

SEQ ID NO: 25 - protein, 17 aa, Artificial sequence
  Synthetic polypeptide.
VISPDGHNTYYADSVKG

SEQ ID NO: 26 - protein, 17 aa, Artificial sequence
  Synthetic polypeptide.
VISPHGRNKYYADSVKG

SEQ ID NO: 27 - protein, 17 aa, Artificial sequence
  Synthetic polypeptide.
VISRRGDNKYYADSVKG

SEQ ID NO: 28 - protein, 17 aa, Artificial sequence
  Synthetic polypeptide.
VISNNGHNKYYADSVKG

SEQ ID NO: 29 - protein, 17 aa, Artificial sequence
  Synthetic polypeptide.
VISPAGPNTYYADSVKG

SEQ ID NO: 30 - protein, 17 aa, Artificial sequence
  Synthetic polypeptide.
VISPSGHNTYYADSVKG

SEQ ID NO: 31 - protein, 17 aa, Artificial sequence
  Synthetic polypeptide.
VISPNGHNTYYADSVKG

SEQ ID NO: 32 - protein, 17 aa, Artificial sequence
  Synthetic polypeptide.
AISPPGHNTYYADSVKG

SEQ ID NO: 33 - protein, 17 aa, Artificial sequence
  Synthetic polypeptide.
VISPTGANTYYADSVKG

SEQ ID NO: 34 - protein, 17 aa, Artificial sequence
  Synthetic polypeptide.
VISPHGSNKYYADSVKG

SEQ ID NO: 35 - protein, 17 aa, Artificial sequence
  Synthetic polypeptide.
VISNNGHNTYYADSVKG

SEQ ID NO: 36 - protein, 17 aa, Artificial sequence
  Synthetic polypeptide.
VISPAGTNTYYADSVKG

SEQ ID NO: 37 - protein, 17 aa, Artificial sequence
  Synthetic polypeptide.
VISPPGHNTYYADSVKG

SEQ ID NO: 38 - protein, 17 aa, Artificial sequence
  Synthetic polypeptide.
VISHDGTNTYYADSVKG

SEQ ID NO: 39 - protein, 17 aa, Artificial sequence
  Synthetic polypeptide.
VISRHGNNKYYADSVKG

SEQ ID NO: 40 - protein, 17 aa, Artificial sequence
  Synthetic polypeptide.
VISYDGSNKYYADSVKG

SEQ ID NO: 41 - protein, 23 aa, Artificial sequence
  Synthetic polypeptide.
ASTGGGGSGGGGSGGGGSGGGGS

SEQ ID NO: 42 - protein, 20 aa, Artificial sequence
  Synthetic polypeptide.
GGGGSGGGGSGGGGSGGGGS

SEQ ID NO: 43 - protein, 15 aa, Artificial sequence
  Synthetic polypeptide.
GGGGSGGGGSGGGGS

SEQ ID NO: 44 - protein, 23 aa, Artificial sequence
  Synthetic polypeptide.
ASTGGGGAGGGGAGGGGAGGGGA

SEQ ID NO: 45 - protein, 20 aa, Artificial sequence
  Synthetic polypeptide.
GGGGAGGGGAGGGGAGGGGA

SEQ ID NO: 46 - protein, 24 aa, Artificial sequence
  Synthetic polypeptide.
TPSHNSHQVPSAGGPTANSGTSGS

SEQ ID NO: 47 - protein, 18 aa, Artificial sequence
  Synthetic polypeptide.
GGSSRSSSSGGGGSGGGG

SEQ ID NO: 48 - protein, 253 aa, Artificial sequence
  Synthetic polypeptide.
QVQLVQSGGGLVQPGGSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 49 - protein, 8 aa, Artificial sequence
  Synthetic polypeptide.
DYKDDDDK

SEQ ID NO: 50 - protein, 4 aa, Artificial sequence
  Synthetic polypeptide.
AGST

SEQ ID NO: 51 - protein, 5 aa, Artificial sequence
  Synthetic polypeptide.
AVDEG

SEQ ID NO: 52 - protein, 10 aa, Artificial sequence
  Synthetic polypeptide.
CDGHNPRSTY

SEQ ID NO: 53 - protein, 9 aa, Artificial sequence
  Synthetic polypeptide.
ADGHNPRST

SEQ ID NO: 54 - protein, 6 aa, Artificial sequence
  Synthetic polypeptide.
GGSGGC

SEQ ID NO: 55 - protein, 254 aa, Artificial sequence
  Synthetic polypeptide.
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSSASTGGGGSGGGGSGGGGSGGGGSQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLG

SEQ ID NO: 56 - DNA, 762 bp, Artificial sequence
  Synthetic nucleic acid.
caagtgcagctggtcgaatccggagggggactggtgcagccgggaggctcactcagactgtcctgcgccgcttcgggcttcactttccgctcgtacgctatgagctgggtccgccaagcccccggaaagggattggaatgggtgtccgccattagcggacggggcgataacacgtactacgcggacagcgtcaaaggcagattcaccattagccgagataacagcaagaataccctgtacctccaaatgaatagcctcagggccgaggacacggctgtgtactactgcgcaaaaatgacctccaacgcatttgcgttcgactactggggacagggaaccctggtcaccgtgtcatcggcatcgactggagggggaggctctggaggagggggatcgggtggcggagggtcgggcggaggaggctcacagtccgtgttgacccaaccgccgtcagtgtccggagcgccagggcagcgggtcaccatctcctgcactggatcgtcatcgaacatcggcgcgggttacggcgtgcactggtatcagcagctgccgggaaccgctcctaagctcctgatctacggcaacactaatcggccatcgggagtgcctgaccgctttagcggtttcaaatccggaacttcagcgagcctggccatcactggtctgcaagccgaggatgaagcggactactactgccaatcgtatgattcctcgctgtccgggtgggtgttcggcgggggaactaagctgaccgtgctgggt

SEQ ID NO: 57 - protein, 253 aa, Artificial sequence
  Synthetic polypeptide.
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTMVTVSSASTGGGGSGGGGSGGGGSGGGGSSSELTQDPAVSVALGQTVSITCQGDSLRSYYASWYQQKPGQAPLLVIYGENNRPSGIPDRFSGSSSGNTASLTITGAQAEDEADYYCNSRDSSGTHLTVFGGGTKLTVLG

SEQ ID NO: 58 - DNA, 759 bp, Artificial sequence
  Synthetic nucleic acid.
caagtgcagctgcagcagtccggagggggagtggtgcagccgggacggtcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccatggtcaccgtgtcatcggcatcgactggagggggaggctctggaggagggggatcgggtggcggagggtcgggcggaggaggctcatcatccgagttgacccaagatccggccgtgtccgtggcgctggggcagactgtctccatcacttgccaaggagactcactgcgctcctactacgcctcgtggtatcagcagaaaccgggacaggctcctctgctcgtgatctacggcgaaaacaatcggccatcgggaatccctgaccgctttagcggttcgagctccggaaacactgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 59 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 60 - protein, 253 aa, Artificial sequence
  Synthetic polypeptide.
QVQLVQSGGGLVQPGGSLRLSCAASGFTFSSYAMHWVRQAPGKGLEWVSVISPAGNNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 61 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccggcgggcaacaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 62 - protein, 253 aa, Artificial sequence
  Synthetic polypeptide.
QVQLVQSGGGLVQPGGSLRLSCAASGFTFSSYAMHWVRQAPGKGLEWVSVISPAGRNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 63 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccggcgggccgcaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 64 - protein, 253 aa, Artificial sequence
  Synthetic polypeptide.
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 65 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccggatggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 66 - protein, 253 aa, Artificial sequence
  Synthetic polypeptide.
QVQLVQSGGGLVQPGGSLRLSCAASGFTFSSYAMHWVRQAPGKGLEWVTVISPHGRNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 67 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgcatggccgcaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 68 - protein, 253 aa, Artificial sequence
  Synthetic polypeptide.
QVQLVQSGGGLVQPGGSLRLSCAASGFTFSSYAMHWVRQAPGKGLEWVSVISRRGDNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 69 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagccgccgcggcgataacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 70 - protein, 253 aa, Artificial sequence
  Synthetic polypeptide.
QVQLVQSGGGLVQPGGSLRLSCAASGFTFSSYAMHWVRQAPGKGLEWVSVISNNGHNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 71 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcaacaacggccataacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 72 - protein, 253 aa, Artificial sequence
  Synthetic polypeptide.
QVQLVQSGGGLVQPGGSLRLSCAASGFTFSSYAMHWVRQAPGKGLEWVSVISPAGPNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 73 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccggcgggcccgaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 74 - protein, 253 aa, Artificial sequence
  Synthetic polypeptide.
QVQLVQSGGGLVQPGGSLRLSCAASGFTFSSYAMHWVRQAPGKGLEWVTVISPSGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 75 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgagcggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 76 - protein, 253 aa, Artificial sequence
  Synthetic polypeptide.
QVQLVQSGGGLVQPGGSLRLSCAASGFTFSSYAMHWVRQAPGKGLEWVTVISPNGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 77 - DNA, 804 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaacggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggtcgtacggtggcggcgcccagtcaccatcatcatcaccactgataa

SEQ ID NO: 78 - protein, 253 aa, Artificial sequence
  Synthetic polypeptide.
QVQLVQSGGGLVQPGGSLRLSCAASGFTFSSYAMHWVRQAPGKGLEWVSAISPPGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 79 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgcgattagcccgccgggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 80 - protein, 253 aa, Artificial sequence
  Synthetic polypeptide.
QVQLVQSGGGLVQPGGSLRLSCAASGFTFSSYAMHWVRQAPGKGLEWVAVISHHGSNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 81 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtggcggtgattagccatcatggcagcaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 82 - protein, 253 aa, Artificial sequence
  Synthetic polypeptide.
QVQLVQSGGGLVQPGGSLRLSCAASGFTFSSYAMHWVRQAPGKGLEWVAGISHPGDNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 83 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtggcgggcattagccatccgggcgataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 84 - protein, 253 aa, Artificial sequence
  Synthetic polypeptide.
QVQLVQSGGGLVQPGGSLRLSCAASGFTFSSYAMHWVRQAPGKGLEWVTVISPTGANTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 85 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgaccggcgcgaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 86 - protein, 253 aa, Artificial sequence
  Synthetic polypeptide.
QVQLVQSGGGLVQPGGSLRLSCAASGFTFSSYAMHWVRQAPGKGLEWVTVISPAGPNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 87 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccggcgggcccgaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 88 - protein, 253 aa, Artificial sequence
  Synthetic polypeptide.
QVQLVQSGGGLVQPGGSLRLSCAASGFTFSSYAMHWVRQAPGKGLEWVSVISPHGSNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 89 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccgcatggcagcaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 90 - protein, 253 aa, Artificial sequence
  Synthetic polypeptide.
QVQLVQSGGGLVQPGGSLRLSCAASGFTFSSYAMHWVRQAPGKGLEWVSVISNNGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 91 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcaacaacggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 92 - protein, 253 aa, Artificial sequence
  Synthetic polypeptide.
QVQLVQSGGGLVQPGGSLRLSCAASGFTFSSYAMHWVRQAPGKGLEWVTVISPPGSNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 93 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgccgggcagcaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 94 - protein, 253 aa, Artificial sequence
  Synthetic polypeptide.
QVQLVQSGGGLVQPGGSLRLSCAASGFTFSSYAMHWVRQAPGKGLEWVSVISPAGTNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 95 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccggcgggcaccaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 96 - protein, 253 aa, Artificial sequence
  Synthetic polypeptide.
QVQLVQSGGGLVQPGGSLRLSCAASGFTFSSYAMHWVRQAPGKGLEWVSVISPPGHNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 97 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgagcgtgattagcccgccgggccataacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 98 - protein, 253 aa, Artificial sequence
  Synthetic polypeptide.
QVQLVQSGGGLVQPGGSLRLSCAASGFTFSSYAMHWVRQAPGKGLEWVTVISHDGTNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 99 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagccatgatggcaccaacacctattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 100 - protein, 253 aa, Artificial sequence
  Synthetic polypeptide.
QVQLVQSGGGLVQPGGSLRLSCAASGFTFSSYAMHWVRQAPGKGLEWVTVISRHGNNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 101 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagccgccacggcaacaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 102 - protein, 253 aa, Artificial sequence
  Synthetic polypeptide.
QVQLVQSGGGLVQPGGSLRLSCAASGFTFSSYAMHWVRQAPGKGLEWVTVISPGGPNKYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 103 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggactggaatgggtgaccgtgattagcccgggcggcccgaacaaatattatgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaacccccgtccgtgtccgtggccccggggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 104 - protein, 253 aa, Artificial sequence
  Synthetic polypeptide.
QVQLVQSGGGLVQPGGSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTLVTVSSASTGGGGSGGGGSGGGGSGGGGSSSELTQDPAVSVALGQTVSITCQGDSLRSYYASWYQQKPGQAPLLVIYGENNRPSGIPDRFSGSSSGNTASLTITGAQAEDEADYYCNSRDSSGTHLTVFGGGTKLTVLG

SEQ ID NO: 105 - DNA, 759 bp, Artificial sequence
  Synthetic nucleic acid.
caagtgcagctggtgcagtccggagggggactggtgcagccgggaggctcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaagatccggccgtgtccgtggcgctggggcagactgtctccatcacttgccaaggagactcactgcgctcctactacgcctcgtggtatcagcagaaaccgggacaggctcctctgctcgtgatctacggcgaaaacaatcggccatcgggaatccctgaccgctttagcggttcgagctccggaaacactgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 106 - protein, 255 aa, Artificial sequence
  Synthetic polypeptide.
QVQLVQSGGGLVQPGGSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTLVTVSSASTGGGGSGGGGSGGGGSGGGGSQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLG

SEQ ID NO: 107 - DNA, 765 bp, Artificial sequence
  Synthetic nucleic acid.
caagtgcagctggtgcagtccggagggggactggtgcagccgggagggtcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcacaatccgtgttgacccaaccgccgtcagtgtccggcgcgcccgggcagcgggtcactatctcctgcaccggatcgtcatcgaacatcggagccggatacggcgtgcactggtatcagcagctgccgggaaccgctcctaaactcctgatctacggcaacactaatcggccatcgggagtgcctgaccgctttagcggtttcaaatccggaacctctgcgagcctggccatcactggtctgcaagccgaggatgaagcggactactactgccagtcgtacgattcctccctctccgggtgggtgttcggcgggggaactaagctgaccgtgctgggt

SEQ ID NO: 108 - protein, 252 aa, Artificial sequence
  Synthetic polypeptide.
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 109 - DNA, 756 bp, Artificial sequence
  Synthetic nucleic acid.
caagtgcagctggtcgaatccggagggggactggtgcagccgggaggatcactcagactgtcctgcgccgcttcgggcttcactttccggtcgtacgctatgtcctgggtccgccaagcccccggaaagggattggaatgggtgtccgcaattagcggacgcggcgataacacttactacgcggacagcgtcaaaggcagattcaccattagccgagataacagcaagaataccctgtacctccaaatgaatagcctcagggccgaggacacggctgtgtactactgcgccaagatgacctcgaacgcatttgcgttcgactactggggacagggaaccctggtcaccgtgtcatcggcatcgactggagggggaggctctggaggagggggatcgggtggcggagggtcgggcggaggaggctcatcatccgagttgacccaaccaccgtcagtgtccgtggccccggggcagactgtcaccatcacttgccaaggagactcactgcgctcctactacgcctcgtggtatcagcagaaaccgggaacggctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 110 - protein, 253 aa, Artificial sequence
  Synthetic polypeptide.
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTMVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 111 - DNA, 759 bp, Artificial sequence
  Synthetic nucleic acid.
caagtgcagctgcagcagtccggagggggagtggtgcagccgggacggtcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccatggtcaccgtgtcatcggcatcgactggagggggaggctctggaggagggggatcgggtggcggagggtcgggcggaggaggctcatcatccgagttgacccaaccgccgtccgtgtccgtggcccccgggcagactgtcactatcacttgccaaggagactcactgcgctcctactacgcctcgtggtatcagcagaaaccgggaaccgctcctaaactcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 112 - protein, 255 aa, Artificial sequence
  Synthetic polypeptide.
QVQLQQSGGGVVQPGRSLRLSCAASGFTFSSYAMHWVRQAPGKGLEWVAVISYDGSNKYYADSVKGRFTISRDNSKNTLYLQMNSLRAEDTAVYYCARASVGATGPFDIWGQGTMVTVSSASTGGGGSGGGGSGGGGSGGGGSQSVLTQPPSVSGAPGQRVTISCTGSSSNIGAGYGVHWYQQLPGTAPKLLIYGNTNRPSGVPDRFSGFKSGTSASLAITGLQAEDEADYYCQSYDSSLSGWVFGGGTKLTVLG

SEQ ID NO: 113 - DNA, 765 bp, Artificial sequence
  Synthetic nucleic acid.
caagtgcagctgcagcagtccggagggggagtggtgcagccgggacggtcactcagactgtcctgcgccgcttcgggcttcactttctcctcgtacgctatgcattgggtccgccaagcccccggaaagggattggaatgggtggcagtgattagctacgacggctcgaacaagtactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccatggtcaccgtgtcatcggcatcgactggagggggaggctctggaggagggggatcgggtggcggagggtcgggcggaggaggctcacaatccgtgttgacccaaccgccgtccgtgtccggagcgcccgggcagcgggtcaccatctcgtgcactggctcctcatcgaacatcggcgcgggctacggcgtgcactggtatcagcagctgccgggaactgctcctaaactcctgatctacggcaacaccaatcggccatcgggagtccctgaccgctttagcggtttcaaatccggaacctctgcgagcctggccatcactggtctgcaagccgaggatgaagcggactactactgccaatcgtatgattcctcactgtccgggtgggtgttcggcgggggaactaagctgaccgtgctgggt

SEQ ID NO: 114 - protein, 252 aa, Artificial sequence
  Synthetic polypeptide.
QVQLVESGGGLVQPGGSLRLSCAASGFTFRSYAMSWVRQAPGKGLEWVSAISGRGDNTYYADSVKGRFTISRDNSKNTLYLQMNSLRAEDTAVYYCAKMTSNAFAFDYWGQGTLVTVSSASTGGGGSGGGGSGGGGSGGGGSSSELTQDPAVSVALGQTVSITCQGDSLRSYYASWYQQKPGQAPLLVIYGENNRPSGIPDRFSGSSSGNTASLTITGAQAEDEADYYCNSRDSSGTHLTVFGGGTKLTVLG

SEQ ID NO: 115 - DNA, 756 bp, Artificial sequence
  Synthetic nucleic acid.
caagtgcagctggtcgaatccggagggggactggtgcagccgggaggctcactcagactgtcctgcgccgcttcgggcttcactttccggtcgtacgctatgtcttgggtccgccaagcccccggaaagggattggaatgggtgtcagcgattagcggacgcggcgacaacacttactacgcggacagcgtcaaaggcagattcaccattagccgagataacagcaagaataccctgtacctccaaatgaatagcctcagggccgaggacacggctgtgtactactgcgcaaagatgacgtccaacgcattcgcgttcgactactggggacagggaaccctggtcaccgtgtcatcggcatcgactggagggggaggctctggaggagggggatcgggtggcggagggtcgggcggaggaggctcatcatccgagttgacccaagatccggccgtgtccgtggcgctggggcagactgtctccatcacttgccaaggagactcactgcgctcctactacgcctcgtggtatcagcagaaaccgggacaggctcctctgctcgtgatctacggcgaaaacaatcggccatcgggaatccctgaccgctttagcggttcgagctccggaaacactgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 116 - protein, 253 aa, Artificial sequence
  Synthetic polypeptide.
QVQLVESGGGLVQPGGSLRLSCAASGFTFSSYAMHWVRQAPGKGLEWVSAISGRGDNTYYADSVKGRFTISRDNSKNTLYLQMNSLRAEDTAVYYCARASVGATGPFDIWGQGTLVTVSSASTGGGGSGGGGSGGGGSGGGGSSSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 117 - DNA, 759 bp, Artificial sequence
  Synthetic nucleic acid.
caagtgcagctggtcgaatccggagggggactggtgcagccgggaggctcactcagactgtcctgcgccgcttcgggcttcactttctcctcgtacgctatgcattgggtccgccaagcccccggaaagggattggaatgggtgtcggccattagcggacggggcgataacacctactacgcggacagcgtcaaaggcagattcaccattagccgagataacagcaagaataccctgtacctccaaatgaatagcctcagggccgaggacacggctgtgtactactgcgcacgcgcgtcagtcggcgcaacgggtccattcgacatctggggacagggaaccctggtcaccgtgtcatcggcatcgactggagggggaggctctggaggagggggatcgggtggcggagggtcgggcggaggaggctcatcatccgagttgacccaaccgccgtccgtgtccgtggcccccgggcagactgtcactatcacttgccaaggagactcactgcgctcctactacgcctcgtggtatcagcagaaaccgggaacggctcctaagctcctgatctacggcgaaaacaatcggccatcgggagtgcctgaccgctttagcggttcgagctccggaacttctgcgagcctgaccatcactggtgcccaagccgaggatgaagcggactactactgcaactcgcgggattcctccgggacccacctgaccgtgttcggcgggggaactaagctgaccgtgctgggt

SEQ ID NO: 118 - protein, 20 aa, Artificial sequence
  Synthetic polypeptide.
MGWSLILLFLVAVATRVLSS

SEQ ID NO: 119 - DNA, 60 bp, Artificial sequence
  Synthetic nucleic acid.
atgggctggtctctgatcctgctgttcctggtggccgtggccacgcgtgtgctctcgagc

SEQ ID NO: 120 - protein, 7 aa, Artificial sequence
  Synthetic polypeptide.
RTVAAPS

SEQ ID NO: 121 - DNA, 21 bp, Artificial sequence
  Synthetic nucleic acid.
cgtacggtggcggcgcccagt

SEQ ID NO: 122 - protein, 7 aa, Artificial sequence
  Synthetic polypeptide.
QPKAAPS

SEQ ID NO: 123 - DNA, 21 bp, Artificial sequence
  Synthetic nucleic acid.
cagccaaaggccgccccgagc

SEQ ID NO: 124 - protein, 6 aa, Artificial sequence
  Synthetic polypeptide.
HHHHHH

SEQ ID NO: 125 - DNA, 18 bp, Artificial sequence
  Synthetic nucleic acid.
caccatcatcatcaccac

SEQ ID NO: 126 - protein, 13 aa, Artificial sequence
  Synthetic polypeptide.
RTVAAPSHHHHHH

SEQ ID NO: 127 - DNA, 39 bp, Artificial sequence
  Synthetic nucleic acid.
cgtacggtggcggcgcccagtcaccatcatcatcaccac

SEQ ID NO: 128 - protein, 13 aa, Artificial sequence
  Synthetic polypeptide.
QPKAAPSHHHHHH

SEQ ID NO: 129 - DNA, 39 bp, Artificial sequence
  Synthetic nucleic acid.
cagccaaaggccgccccgagccaccatcatcatcaccac

SEQ ID NO: 130 - DNA, 18 bp, Artificial sequence
  Synthetic nucleic acid.
ggcggatccggcggttgc

SEQ ID NO: 131 - protein, 537 aa, Artificial sequence
  Synthetic polypeptide.
MAWRLWWLLLLLLLLWPMVWAQGKEVVLLDFAAAGGELGWLTHPYGKGWDLMQNIMNDMPIYMYSVCNVMSGDQDNWLRTNWVYRGEAERIFIELKFTVRDCNSFPGGASSCKETFNLYYAESDLDYGTNFQKRLFTKIDTIAPDEITVSSDFEARHVKLNVEERSVGPLTRKGFYLAFQDIGACVALLSVRVYYKKCPELLQGLAHFPETIAGSDAPSLATVAGTCVDHAVVPPGGEEPRMHCAVDGEWLVPIGQCLCQAGYEKVEDACQACSPGFFKFEASESPCLECPEHTLPSPEGATSCECEEGFFRAPQDPASMPCTRPPSAPHYLTAVGMGAKVELRWTPPQDSGGREDIVYSVTCEQCWPESGECGPCEASVRYSEPPHGLTRTSVTVSDLEPHMNYTFTVEARNGVSGLVTSRSFRTASVSINQTEPPKVRLEGRSTTSLSVSWSIPPPQQSRVWKYEVTYRKKGDSNSYNVRRTEGFSVTLDDLAPDTTYLVQVQALTQEGQGAGSKVHEFQTLSPEGSGNHHHHHH

SEQ ID NO: 132 - DNA, 1617 bp, Artificial sequence
  Synthetic nucleic acid.
atggcgtggcgcctgtggtggctgctgctgctgctgctgctgctgtggccgatggtgtgggcgcagggcaaagaagtggtgctgctggattttgcggcggcgggcggcgaactgggctggctgacccatccgtatggcaaaggctgggatctgatgcagaacattatgaacgatatgccgatttatatgtatagcgtgtgcaacgtgatgagcggcgatcaggataactggctgcgcaccaactgggtgtatcgcggcgaagcggaacgcatttttattgaactgaaatttaccgtgcgcgattgcaacagctttccgggcggcgcgagcagctgcaaagaaacctttaacctgtattatgcggaaagcgatctggattatggcaccaactttcagaaacgcctgtttaccaaaattgataccattgcgccggatgaaattaccgtgagcagcgattttgaagcgcgccatgtgaaactgaacgtggaagaacgcagcgtgggcccgctgacccgcaaaggcttttatctggcgtttcaggatattggcgcgtgcgtggcgctgctgagcgtgcgcgtgtattataaaaaatgcccggaactgctgcagggcctggcgcattttccggaaaccattgcgggcagcgatgcgccgagcctggcgaccgtggcgggcacctgcgtggatcatgcggtggtgccgccgggcggcgaagaaccgcgcatgcattgcgcggtggatggcgaatggctggtgccgattggccagtgcctgtgccaggcgggctatgaaaaagtggaagatgcgtgccaggcgtgcagcccgggcttttttaaatttgaagcgagcgaaagcccgtgcctggaatgcccggaacataccctgccgagcccggaaggcgcgaccagctgcgaatgcgaagaaggcttttttcgcgcgccgcaggatccggcgagcatgccgtgcacccgcccgccgagcgcgccgcattatctgaccgcggtgggcatgggcgcgaaagtggaactgcgctggaccccgccgcaggatagcggcggccgcgaagatattgtgtatagcgtgacctgcgaacagtgctggccggaaagcggcgaatgcggcccgtgcgaagcgagcgtgcgctatagcgaaccgccgcatggcctgacccgcaccagcgtgaccgtgagcgatctggaaccgcatatgaactatacctttaccgtggaagcgcgcaacggcgtgagcggcctggtgaccagccgcagctttcgcaccgcgagcgtgagcattaaccagaccgaaccgccgaaagtgcgcctggaaggccgcagcaccaccagcctgagcgtgagctggagcattccgccgccgcagcagagccgcgtgtggaaatatgaagtgacctatcgcaaaaaaggcgatagcaacagctataacgtgcgccgcaccgaaggctttagcgtgaccctggatgatctggcgccggataccacctatctggtgcaggtgcaggcgctgacccaggaaggccagggcgcgggcagcaaagtgcatgaatttcagaccctgagcccggaaggcagcggcaaccatcatcatcatcatcattgataa

SEQ ID NO: 133 - protein, 120 aa, Artificial sequence
  synthetic polypeptide
QVQLVQSGGGLVQPGGSLRLSCAASGFTFSSYAMHWVRQAPGKGLEWVSVISPAGNNTYYADSVKGRFTISRDNSKNTLYLQMNSLRAEDTAVYYCARASVGATGPFDIWGQGTLVTVSS

SEQ ID NO: 134 - protein, 110 aa, Artificial sequence
  synthetic polypeptide
SSELTQPPSVSVAPGQTVTITCQGDSLRSYYASWYQQKPGTAPKLLIYGENNRPSGVPDRFSGSSSGTSASLTITGAQAEDEADYYCNSRDSSGTHLTVFGGGTKLTVLG

SEQ ID NO: 135 - protein, 120 aa, Artificial sequence
  synthetic polypeptide
QVQLVQSGGGLVQPGGSLRLSCAASGFTFSSYAMHWVRQAPGKGLEWVSVISPAGRNKYYADSVKGRFTISRDNSKNTLYLQMNSLRAEDTAVYYCARASVGATGPFDIWGQGTLVTVSS

SEQ ID NO: 136 - protein, 110 aa, Artificial sequence; identical to SEQ ID NO: 134
  synthetic polypeptide

SEQ ID NO: 137 - protein, 120 aa, Artificial sequence
  synthetic polypeptide
QVQLVQSGGGLVQPGGSLRLSCAASGFTFSSYAMHWVRQAPGKGLEWVTVISPDGHNTYYADSVKGRFTISRDNSKNTLYLQMNSLRAEDTAVYYCARASVGATGPFDIWGQGTLVTVSS

SEQ ID NO: 138 - protein, 110 aa, Artificial sequence; identical to SEQ ID NO: 134
  synthetic polypeptide

SEQ ID NO: 139 - protein, 120 aa, Artificial sequence
  synthetic polypeptide
QVQLVQSGGGLVQPGGSLRLSCAASGFTFSSYAMHWVRQAPGKGLEWVTVISPHGRNKYYADSVKGRFTISRDNSKNTLYLQMNSLRAEDTAVYYCARASVGATGPFDIWGQGTLVTVSS

SEQ ID NO: 140 - protein, 110 aa, Artificial sequence; identical to SEQ ID NO: 134
  synthetic polypeptide

SEQ ID NO: 141 - protein, 120 aa, Artificial sequence
  synthetic polypeptide
QVQLVQSGGGLVQPGGSLRLSCAASGFTFSSYAMHWVRQAPGKGLEWVSVISRRGDNKYYADSVKGRFTISRDNSKNTLYLQMNSLRAEDTAVYYCARASVGATGPFDIWGQGTLVTVSS

SEQ ID NO: 142 - protein, 110 aa, Artificial sequence; identical to SEQ ID NO: 134
  synthetic polypeptide

SEQ ID NO: 143 - protein, 120 aa, Artificial sequence
  synthetic polypeptide
QVQLVQSGGGLVQPGGSLRLSCAASGFTFSSYAMHWVRQAPGKGLEWVSVISNNGHNKYYADSVKGRFTISRDNSKNTLYLQMNSLRAEDTAVYYCARASVGATGPFDIWGQGTLVTVSS

SEQ ID NO: 144 - protein, 110 aa, Artificial sequence; identical to SEQ ID NO: 134
  synthetic polypeptide

SEQ ID NO: 145 - protein, 120 aa, Artificial sequence
  synthetic polypeptide
QVQLVQSGGGLVQPGGSLRLSCAASGFTFSSYAMHWVRQAPGKGLEWVSVISPAGPNTYYADSVKGRFTISRDNSKNTLYLQMNSLRAEDTAVYYCARASVGATGPFDIWGQGTLVTVSS

SEQ ID NO: 146 - protein, 110 aa, Artificial sequence; identical to SEQ ID NO: 134
  synthetic polypeptide

SEQ ID NO: 147 - protein, 120 aa, Artificial sequence
  synthetic polypeptide
QVQLVQSGGGLVQPGGSLRLSCAASGFTFSSYAMHWVRQAPGKGLEWVTVISPSGHNTYYADSVKGRFTISRDNSKNTLYLQMNSLRAEDTAVYYCARASVGATGPFDIWGQGTLVTVSS

SEQ ID NO: 148 - protein, 110 aa, Artificial sequence; identical to SEQ ID NO: 134
  synthetic polypeptide

SEQ ID NO: 149 - protein, 120 aa, Artificial sequence
  synthetic polypeptide
QVQLVQSGGGLVQPGGSLRLSCAASGFTFSSYAMHWVRQAPGKGLEWVTVISPNGHNTYYADSVKGRFTISRDNSKNTLYLQMNSLRAEDTAVYYCARASVGATGPFDIWGQGTLVTVSS

SEQ ID NO: 150 - protein, 110 aa, Artificial sequence; identical to SEQ ID NO: 134
  synthetic polypeptide

SEQ ID NO: 151 - protein, 120 aa, Artificial sequence
  synthetic polypeptide
QVQLVQSGGGLVQPGGSLRLSCAASGFTFSSYAMHWVRQAPGKGLEWVSAISPPGHNTYYADSVKGRFTISRDNSKNTLYLQMNSLRAEDTAVYYCARASVGATGPFDIWGQGTLVTVSS

SEQ ID NO: 152 - protein, 110 aa, Artificial sequence; identical to SEQ ID NO: 134
  synthetic polypeptide

SEQ ID NO: 153 - protein, 120 aa, Artificial sequence
  synthetic polypeptide
QVQLVQSGGGLVQPGGSLRLSCAASGFTFSSYAMHWVRQAPGKGLEWVAVISHHGSNTYYADSVKGRFTISRDNSKNTLYLQMNSLRAEDTAVYYCARASVGATGPFDIWGQGTLVTVSS

SEQ ID NO: 154 - protein, 110 aa, Artificial sequence; identical to SEQ ID NO: 134
  synthetic polypeptide

SEQ ID NO: 155 - protein, 120 aa, Artificial sequence
  synthetic polypeptide
QVQLVQSGGGLVQPGGSLRLSCAASGFTFSSYAMHWVRQAPGKGLEWVAGISHPGDNTYYADSVKGRFTISRDNSKNTLYLQMNSLRAEDTAVYYCARASVGATGPFDIWGQGTLVTVSS

SEQ ID NO: 156 - protein, 110 aa, Artificial sequence; identical to SEQ ID NO: 134
  synthetic polypeptide

SEQ ID NO: 157 - protein, 120 aa, Artificial sequence
  synthetic polypeptide
QVQLVQSGGGLVQPGGSLRLSCAASGFTFSSYAMHWVRQAPGKGLEWVTVISPTGANTYYADSVKGRFTISRDNSKNTLYLQMNSLRAEDTAVYYCARASVGATGPFDIWGQGTLVTVSS

SEQ ID NO: 158 - protein, 110 aa, Artificial sequence; identical to SEQ ID NO: 134
  synthetic polypeptide

SEQ ID NO: 159 - protein, 120 aa, Artificial sequence
  synthetic polypeptide
QVQLVQSGGGLVQPGGSLRLSCAASGFTFSSYAMHWVRQAPGKGLEWVTVISPAGPNKYYADSVKGRFTISRDNSKNTLYLQMNSLRAEDTAVYYCARASVGATGPFDIWGQGTLVTVSS

SEQ ID NO: 160 - protein, 110 aa, Artificial sequence; identical to SEQ ID NO: 134
  synthetic polypeptide

SEQ ID NO: 161 - protein, 120 aa, Artificial sequence
  synthetic polypeptide
QVQLVQSGGGLVQPGGSLRLSCAASGFTFSSYAMHWVRQAPGKGLEWVSVISPHGSNKYYADSVKGRFTISRDNSKNTLYLQMNSLRAEDTAVYYCARASVGATGPFDIWGQGTLVTVSS

SEQ ID NO: 162 - protein, 110 aa, Artificial sequence; identical to SEQ ID NO: 134
  synthetic polypeptide

SEQ ID NO: 163 - protein, 120 aa, Artificial sequence
  synthetic polypeptide
QVQLVQSGGGLVQPGGSLRLSCAASGFTFSSYAMHWVRQAPGKGLEWVSVISNNGHNTYYADSVKGRFTISRDNSKNTLYLQMNSLRAEDTAVYYCARASVGATGPFDIWGQGTLVTVSS

SEQ ID NO: 164 - protein, 110 aa, Artificial sequence; identical to SEQ ID NO: 134
  synthetic polypeptide

SEQ ID NO: 165 - protein, 120 aa, Artificial sequence
  synthetic polypeptide
QVQLVQSGGGLVQPGGSLRLSCAASGFTFSSYAMHWVRQAPGKGLEWVTVISPPGSNKYYADSVKGRFTISRDNSKNTLYLQMNSLRAEDTAVYYCARASVGATGPFDIWGQGTLVTVSS

SEQ ID NO: 166 - protein, 110 aa, Artificial sequence; identical to SEQ ID NO: 134
  synthetic polypeptide

SEQ ID NO: 167 - protein, 120 aa, Artificial sequence
  synthetic polypeptide
QVQLVQSGGGLVQPGGSLRLSCAASGFTFSSYAMHWVRQAPGKGLEWVSVISPAGTNTYYADSVKGRFTISRDNSKNTLYLQMNSLRAEDTAVYYCARASVGATGPFDIWGQGTLVTVSS

SEQ ID NO: 168 - protein, 110 aa, Artificial sequence; identical to SEQ ID NO: 134
  synthetic polypeptide

SEQ ID NO: 169 - protein, 120 aa, Artificial sequence
  synthetic polypeptide
QVQLVQSGGGLVQPGGSLRLSCAASGFTFSSYAMHWVRQAPGKGLEWVSVISPPGHNTYYADSVKGRFTISRDNSKNTLYLQMNSLRAEDTAVYYCARASVGATGPFDIWGQGTLVTVSS

SEQ ID NO: 170 - protein, 110 aa, Artificial sequence; identical to SEQ ID NO: 134
  synthetic polypeptide

SEQ ID NO: 171 - protein, 120 aa, Artificial sequence
  synthetic polypeptide
QVQLVQSGGGLVQPGGSLRLSCAASGFTFSSYAMHWVRQAPGKGLEWVTVISHDGTNTYYADSVKGRFTISRDNSKNTLYLQMNSLRAEDTAVYYCARASVGATGPFDIWGQGTLVTVSS

SEQ ID NO: 172 - protein, 110 aa, Artificial sequence; identical to SEQ ID NO: 134
  synthetic polypeptide

SEQ ID NO: 173 - protein, 120 aa, Artificial sequence
  synthetic polypeptide
QVQLVQSGGGLVQPGGSLRLSCAASGFTFSSYAMHWVRQAPGKGLEWVTVISRHGNNKYYADSVKGRFTISRDNSKNTLYLQMNSLRAEDTAVYYCARASVGATGPFDIWGQGTLVTVSS

SEQ ID NO: 174 - protein, 110 aa, Artificial sequence; identical to SEQ ID NO: 134
  synthetic polypeptide

SEQ ID NO: 175 - protein, 120 aa, Artificial sequence
  synthetic polypeptide
QVQLVQSGGGLVQPGGSLRLSCAASGFTFSSYAMHWVRQAPGKGLEWVTVISPGGPNKYYADSVKGRFTISRDNSKNTLYLQMNSLRAEDTAVYYCARASVGATGPFDIWGQGTLVTVSS

SEQ ID NO: 176 - protein, 110 aa, Artificial sequence; identical to SEQ ID NO: 134
  synthetic polypeptide

SEQ ID NO: 177 - protein, 10 aa, Artificial sequence
  synthetic polypeptide
SVISPAGNNT

SEQ ID NO: 178 - protein, 10 aa, Artificial sequence
  synthetic polypeptide
SVISPAGRNK

SEQ ID NO: 179 - protein, 10 aa, Artificial sequence
  synthetic polypeptide
TVISPDGHNT

SEQ ID NO: 180 - protein, 10 aa, Artificial sequence
  synthetic polypeptide
TVISPHGRNK

SEQ ID NO: 181 - protein, 10 aa, Artificial sequence
  synthetic polypeptide
SVISRRGDNK

SEQ ID NO: 182 - protein, 10 aa, Artificial sequence
  synthetic polypeptide
SVISNNGHNK

SEQ ID NO: 183 - protein, 10 aa, Artificial sequence
  synthetic polypeptide
SVISPAGPNT

SEQ ID NO: 184 - protein, 10 aa, Artificial sequence
  synthetic polypeptide
TVISPSGHNT

SEQ ID NO: 185 - protein, 10 aa, Artificial sequence
  synthetic polypeptide
TVISPNGHNT

SEQ ID NO: 186 - protein, 10 aa, Artificial sequence
  synthetic polypeptide
SAISPPGHNT

SEQ ID NO: 187 - protein, 10 aa, Artificial sequence
  synthetic polypeptide
AVISHHGSNT

SEQ ID NO: 188 - protein, 10 aa, Artificial sequence
  synthetic polypeptide
TVISPTGANT

SEQ ID NO: 189 - protein, 10 aa, Artificial sequence
  synthetic polypeptide
TVISPAGPNK

SEQ ID NO: 190 - protein, 10 aa, Artificial sequence
  synthetic polypeptide
SVISPHGSNK

SEQ ID NO: 191 - protein, 10 aa, Artificial sequence
  synthetic polypeptide
SVISNNGHNT

SEQ ID NO: 192 - protein, 10 aa, Artificial sequence
  synthetic polypeptide
TVISPPGSNK

SEQ ID NO: 193 - protein, 10 aa, Artificial sequence
  synthetic polypeptide
SVISPAGTNT

SEQ ID NO: 194 - protein, 10 aa, Artificial sequence
  synthetic polypeptide
SVISPPGHNT

SEQ ID NO: 195 - protein, 10 aa, Artificial sequence
  synthetic polypeptide
TVISHDGTNT

SEQ ID NO: 196 - protein, 10 aa, Artificial sequence
  synthetic polypeptide
TVISRHGNNK

SEQ ID NO: 197 - protein, 10 aa, Artificial sequence
  synthetic polypeptide
TVISPGGPNK

SEQ ID NO: 198 - protein, 10 aa, Artificial sequence
  synthetic polypeptide
  MISC_FEATURE (1)..(1): Xaa is S, T or A.
  MISC_FEATURE (2)..(2): Xaa is A, or V.
  MISC_FEATURE (5)..(5): Xaa is P, R, N or H.
  MISC_FEATURE (6)..(6): Xaa is any amino acid, and in some embodiments Xaa is A, D, H, N, P, S or T.
  MISC_FEATURE (8)..(8): Xaa is any amino acid and in some embodiments Xaa is A, R, H, N or P.
  MISC_FEATURE (10)..(10): Xaa is K or T.
XXISXXGXNX

SEQ ID NO: 199 - protein, 10 aa, Artificial sequence
  synthetic polypeptide
  MISC_FEATURE (1)..(1): Xaa is S or T.
  MISC_FEATURE (5)..(5): Xaa is P, R, or N.
  MISC_FEATURE (6)..(6): Xaa is any amino acid, and in some embodiments Xaa is A, D, H, N, P, S or T.
  MISC_FEATURE (8)..(8): Xaa is H, N, R, T, A, D, P, or S.
  MISC_FEATURE (10)..(10): Xaa is K or T.
XVISXXGXNX

SEQ ID NO: 200 - protein, 10 aa, Artificial sequence
  synthetic polypeptide
  MISC_FEATURE (1)..(1): Xaa is S or T.
  MISC_FEATURE (5)..(5): Xaa is P, R, or N.
  MISC_FEATURE (6)..(6): Xaa is any amino acid, and in some embodiments is A, D, H, N, P, S or T.
  MISC_FEATURE (8)..(8): Xaa is H, N, R, or T.
  MISC_FEATURE (10)..(10): Xaa is T or K.
XVISXXGXNX

SEQ ID NO: 201 - protein, 10 aa, Artificial sequence
  synthetic polypeptide
  MISC_FEATURE (1)..(1): Xaa is S or T.
  MISC_FEATURE (2)..(2): Xaa is V.
  MISC_FEATURE (5)..(5): Xaa is P.
  MISC_FEATURE (6)..(6): Xaa is any amino acid, and in some embodiments is A, D, H, N, P, S or T.
  MISC_FEATURE (8)..(8): Xaa is H, N, R, or T, and in some embodiments is A, R, H, N or P.
  MISC_FEATURE (10)..(10): Xaa is T or K.
XXISXXGXNX

SEQ ID NO: 202 - protein, 13 aa, Artificial sequence
  synthetic polypeptide
WVRQAPGKGLEWV